SEQUENCE LISTING (11 sequences)

SEQ ID NO: 1 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 3 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 4 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 5 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 6 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 7 - protein, 1297 aa, Clostridium botulinum
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 8 - protein, 1285 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTQFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIIGNTNDTRVRGENVLYFNTTIDNKQYSLGMYKPSRNLGTDLVPLGALDQPMDEIRKYGSFIIQPCNTFDYYASQLFLSSNATTNRIGILSIGSYSFKLGDDYWFNHEYLIPVIKIEHYASLLESTSTHWVFVPASE

SEQ ID NO: 9 - protein, 1268 aa, Clostridium botulinum
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESKGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYDNLLSNYTAIASRLSQVNINNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLVVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLKAEYNIYSIKEELNKKVSLAMQNIDRFLTESSISYLMKLINEAKINKLSEYDKRVNQYLLNYILENSSTLGTSSVQELNNLVSNTLNNSIPFELSEYTNDKILISYFNRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 10 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 11 - protein, 1296 aa, Clostridium botulinum [sequence omitted]

(19) United States
(12) Patent Application Publication
(10) Pub. No.: US 2023/0242962 A1
(43) Pub. Date: Aug. 3, 2023

(54) CONTROLLING OPERATION OF A BIOREACTOR VESSEL

(71) Applicant: IPSEN BIOPHARM LIMITED, Wrexham (GB)

(72) Inventor: Stanislav PEPELIAEV, Wrexham (GB)

(21) Appl. No.: 18/003,570

(22) PCT Filed: Jul. 15, 2021
(86) PCT No.: PCT/GB2021/051820

§ 371 Date: Dec. 28, 2022

(30) Foreign Application Priority Data
Jul. 15, 2020 (GB) 2010934.4

(51) Int. Cl. C12P 21/02; C12N 9/52; C12M 1/36; C12M 1/34; C12M 1/02; C12M 1/06

(52) U.S. Cl. CPC C12P 21/02; C12N 9/52; C12Y 304/24069; C12M 41/48; C12M 41/34; C12M 41/26; C12M 41/42; C12M 41/22; C12M 27/02

(57) ABSTRACT

There is provided a method of controlling operation of a fed batch process in a bioreactor vessel, comprising transitioning from a batch phase to a production phase in dependence on a relationship between an oxygen supply parameter O and a dissolved oxygen value DO.

### FIELD OF THE INVENTION

The present disclosure relates to controlling operation of a bioprocess in a bioreactor system. The disclosure is particularly, but not exclusively, applicable to a method and device for controlling operation of a bioprocess in a bioreactor vessel, including transitioning from different phases of operation of the bioprocess and/or controlling process conditions within the bioreactor vessel.

### BACKGROUND TO THE DISCLOSURE

In order to ensure a high efficiency/yield of a bioprocess in a bioreactor system, process conditions (such as temperature, dissolved oxygen concentration, or pH) are typically controlled to specific setpoints. Many existing bioreactor controllers use single-input single-output (SISO) control loops for each process condition to achieve this.

Bioprocesses controlled by existing bioreactor controllers can lack reproducibility and consistency between batches can be poor. Many existing bioreactor controllers are unable to transition optimally between phases in the bioprocess occurring in the bioreactor vessel, for example, in the case of a batch-fed fermentation process, between: a batch phase (also referred to as an exponential phase or a growth phase) during which cells grow and propagate at an increasing rate; and a production phase (also referred to as an expression phase or a fed-batch phase) during which population growth slows or ceases and the target bioprocess is performed. The transitioning between phases is often a complex and involved process. Accordingly, many existing bioreactor controllers are unable to transition optimally in an automated manner, which may inherently introduce inaccuracy, deviation from optimum process conditions, and an undesirable degree of variability between batches. Control of transitioning may be particularly important as each phase may have different associated desirable process conditions and so require different control (e.g. different setpoints for the conditions, or entirely different control signals) of the bioreactor system to achieve these conditions. Thus, any delay between the actual phase transitioning and the optimum phase transition may result in inappropriate/suboptimal control of the bioreactor in the meantime (e.g. incorrect process condition setpoints being used).

The present disclosure seeks to enable more effective control in a bioreactor system.

### SUMMARY OF THE DISCLOSURE

Aspects of the disclosure are set out in the accompanying claims.

According to an aspect of the disclosure there is provided a method of controlling operation of a fed batch process in a bioreactor vessel, comprising transitioning from a batch phase to a production phase in dependence on a relationship between an oxygen supply parameter O and a dissolved oxygen value DO.

Using a relationship may result in more responsive/faster control and an increased operational output (process condition) range. A process condition may in fact be affected by multiple control parameters, and a relationship can enable particularly effective transitioning between phases. By considering a relationship between an oxygen supply parameter O and a dissolved oxygen value DO, an approximation or estimate of an oxygen consumption rate of the process can be provided, which can be particularly effective for controlling transitioning between phases.

The oxygen supply parameter O may be determined in dependence on one or more of: an agitation speed; a gas supply rate; and an oxygen supply concentration. The oxygen supply parameter O may be a sum or product of two or more of: an agitation speed value, a gas supply rate value, and an oxygen supply concentration value.

Preferably, the relationship is a mathematical relationship.

A relationship may be defined by an aggregate parameter (in other words, the method may comprise transitioning from a batch phase to a production phase in dependence on an aggregate parameter, the aggregate parameter depending on (both) an oxygen supply parameter O and a dissolved oxygen value DO). Preferably, the aggregate parameter increases with increasing oxygen supply O and decreases with increasing dissolved oxygen DO. The relationship (aggregate parameter) may be a ratio

$$\frac{o}{D O} .$$

The relationship may be a difference, an exponential relationship, a trigonometric relationship, or a logarithmic relationship. For effective transitioning the method may comprise transitioning from a batch phase to a production phase when the ratio

$$\frac{o}{D O}$$

falls below a threshold k. For avoidance of premature transitioning the method may comprise transitioning from a batch phase to a production phase when the ratio

$$\frac{o}{D O}$$

falls below a threshold k only if the ratio

$$\frac{o}{D O}$$

has previously exceeded a threshold j. For avoidance of premature transitioning the method may comprise transitioning from a batch phase to a production phase when the ratio

$$\frac{o}{D O}$$

falls below a threshold k only if the ratio

$$\frac{o}{D O}$$

has previously exceeded a threshold j for at least a predetermined period of time. Occasional spikes may occur in the observed process conditions, without being caused by an underlying process change such as carbon source exhaustion. Such occasional spikes can cause the controller to transition phases prematurely, resulting in lower yield and effectiveness of the bioprocess.

Optionally, the method comprises transitioning from a batch phase to a production phase in dependence on the relative values of an oxygen supply parameter O and of a dissolved oxygen DO (i.e. in dependence on a comparison of the values of an oxygen supply parameter O and of a dissolved oxygen DO), optionally wherein the oxygen supply parameter O value and/or the dissolved oxygen DO value is scaled by a factor.

The process may be a protein expression process. A target protein expressed in the production phase may be any protein, for example produced by a bacterial or yeast host or any protein obtainable by recombinant protein technology. A target protein expressed in the production phase may be a recombinant protein. Non-limiting examples of proteins include Clostridium neurotoxins such as a native Botulinum neurotoxin or recombinant Botulinum neurotoxin or recombinant antibodies or recombinant hormones and the like.

The method may comprise controlling one or more actuators. The method may comprise receiving sensor data from one or more sensors. The method may comprise: controlling one or more actuators to provide a first set of process conditions during the batch phase; and controlling the actuators to provide a second set of process conditions during the production phase, with the first and second set of process conditions being different at least in part. Transitioning from the batch phase to the production phase may comprise changing one or more process condition setpoints. The one or more process condition setpoints may include one or more of: a dissolved oxygen setpoint; and a temperature setpoint. Transitioning from the batch phase to the production phase may comprise one or more of: providing feed to the bioreactor vessel; and providing inducer to the bioreactor vessel.

For optimising process conditions transitioning from the batch phase to the production phase may comprise gradually changing over a predetermined period of time one or more process condition setpoints from a batch phase setpoint to a production phase setpoint.

For optimising process conditions the method may further comprise controlling an oxygen supply rate and/or a gas supply rate and/or an oxygen supply concentration proportional to the agitation speed at least in a (sub-)range of the agitation.

For smooth process control the method may further comprise controlling an agitation speed in dependence on a rate of change of dissolved oxygen. The rate of change of dissolved oxygen may be determined in dependence on a first dissolved oxygen measured value, a second, preceding, dissolved oxygen measured value, and optionally a difference between measurement times of the first and second dissolved oxygen measured values.

For smooth process control the method may further comprise gradually transitioning over a predetermined period of time from the production phase to a termination phase. The transitioning may comprise one or more of: reducing a feed supply and/or an oxygen supply to the bioreactor vessel; transitioning one or more process conditions from a production phase setpoint to a termination setpoint; and reducing agitation in the bioreactor vessel. For improved process conditions the transitioning may comprise reducing a feed supply to the bioreactor vessel and transitioning a temperature from a production phase setpoint to a termination setpoint, wherein the temperature is transitioned at a rate proportional to the rate at which the feed supply is reduced. For improved process conditions the transitioning may comprise first reducing a feed supply to the bioreactor vessel and transitioning a temperature from a production phase setpoint to a termination setpoint, and then reducing an agitation.

The dissolved oxygen value DO is preferably a measured dissolved oxygen value or a value dependent on a measured dissolved oxygen value. The dissolved oxygen value DO may be received from a dissolved oxygen sensor.

The method may further comprise adapting an agitation and an oxygen supply simultaneously or near-simultaneously in dependence on a dissolved oxygen setpoint and a measured dissolved oxygen.

According to another aspect of the disclosure there is provided a computer programme product comprising instructions which, when executed by a computer, cause the computer to control operation of a fed batch process in a bioreactor vessel, comprising: determining a relationship, preferably a ratio

$$\frac{o}{D O} ,$$

between an oxygen supply parameter O and a dissolved oxygen value DO; and transitioning from a batch phase to a production phase in dependence on the relationship, and preferably the ratio

$$\frac{o}{D O} .$$

The computer programme product may comprise instructions which, when executed by a computer, cause the computer to control operation of a fed batch process in a bioreactor vessel according to any method as aforementioned.

According to another aspect of the disclosure there is provided a device adapted to control operation of a fed batch process in a bioreactor vessel, the device comprising: means for determining a relationship, preferably a ratio

$$\frac{o}{D O} ,$$

between an oxygen supply parameter O and a dissolved oxygen value DO; and a control output adapted to transition the process from a batch phase to a production phase in dependence on the relationship, and preferably the ratio

$$\frac{o}{D O} .$$

The device may further be adapted to and/or comprise means adapted to control operation of a fed batch process in a bioreactor vessel according to any method as aforementioned. The device may further include a bioreactor vessel, one or more sensors for sensing process conditions of a fed batch process in the bioreactor vessel, and one or more actuators adapted to affect process conditions in the bioreactor vessel.

According to another aspect of the disclosure there is provided a method of controlling operation of a bioprocess in a bioreactor vessel, comprising adapting an agitation in dependence on a rate of change of dissolved oxygen. This can enable better control of the bioprocess, with less deviation of the actual process conditions from the desired process conditions.

The agitation may be adapted in dependence on a dissolved oxygen setpoint value, a first dissolved oxygen measured value, a second, preceding, dissolved oxygen measured value, and, optionally, a difference between measurement times of the first and second dissolved oxygen values. The method may further comprise adapting an oxygen supply proportional to the agitation at least in a (sub-)range of the agitation.

Preferably, the agitation is adapted further in dependence on the size of the vessel (e.g. a working volume of the vessel), and optionally a maximum and/or minimum permitted agitation speed. This may allow preventing overshooting, in particular for larger vessels.

According to another aspect of the disclosure there is provided a computer programme product comprising instructions which, when executed by a computer, cause the computer to control operation of a bioprocess in a bioreactor vessel, comprising adapting an agitation in dependence on a rate of change of dissolved oxygen. The computer programme product may comprise instructions which, when executed by a computer, cause the computer to control operation of a bioreactor vessel according to any method as aforementioned.

According to another aspect of the disclosure there is provided a device adapted to control operation of a bioprocess in a bioreactor vessel, the device comprising: means for adapting an agitation in dependence on a rate of change of dissolved oxygen. The device may further be adapted to and/or comprise means adapted to control operation of a bioprocess in a bioreactor vessel according to any method as aforementioned.

According to another aspect of the disclosure there is provided a method of controlling operation of a bioprocess in a bioreactor vessel, comprising adapting an acid supply and/or a base supply in dependence on a current pH value and a preceding pH value. Preferably the acid supply and/or base supply is adapted to increase exponentially with a time during which a measured pH is not at a pH setpoint. This can enable swift and effective pH control, while reducing overshoots in pH control.

According to another aspect of the disclosure there is provided a computer programme product comprising instructions which, when executed by a computer, cause the computer to control operation of a bioprocess in a bioreactor vessel, comprising adapting an acid supply and/or a base supply in dependence on a current pH value and a preceding pH value. Preferably the acid supply and/or base supply is adapted to increase exponentially with a time during which a measured pH is not at a pH setpoint. This can enable swift and effective pH control, while reducing overshoots in pH control. The computer programme product may comprise instructions which, when executed by a computer, cause the computer to control operation of a bioreactor vessel according to any method as aforementioned.

According to another aspect of the disclosure there is provided a device adapted to control operation of a bioprocess in a bioreactor vessel, the device comprising: means for adapting an acid supply and/or a base supply in dependence on a current pH value and a preceding pH value. Preferably the acid supply and/or base supply is adapted to increase exponentially with a time during which a measured pH is not at a pH setpoint. This can enable swift and effective pH control, while reducing overshoots in pH control. The device may further be adapted to and/or comprise means adapted to control operation of a bioprocess in a bioreactor vessel according to any method as aforementioned.

According to another aspect of the disclosure there is provided a method of controlling operation of a bioprocess in a bioreactor vessel, comprising adapting an acid supply and/or a base supply in dependence on a current pH value, a preceding pH value, and a pH setpoint.

Preferably, the acid supply and/or base supply is adapted to increase exponentially with a time during which a measured pH is not at the pH setpoint.

Preferably, the acid supply and/or base supply is adapted to increase exponentially with a time during which a measured pH (gradient) tends away from the pH setpoint (e.g. time during which the measured pH gradient is negative (or zero) if the measured pH is below the pH setpoint, or time during which the measured pH gradient is positive (or zero) if the measured pH is above the pH setpoint). In other words, the acid supply and/or base supply is preferably adapted to increase exponentially when the current pH value is greater (or lesser) than both the preceding pH value and the pH setpoint. This can enable swift and effective pH control, while reducing overshoots in pH control.

Preferably, the acid supply and/or base supply is scaled in dependence on the difference between the current pH value and the pH setpoint.

According to another aspect of the disclosure there is provided a computer programme product comprising instructions which, when executed by a computer, cause the computer to control operation of a bioprocess in a bioreactor vessel, comprising adapting an acid supply and/or a base supply in dependence on a current pH value, a preceding pH value, and a pH setpoint. The computer programme product may comprise instructions which, when executed by a computer, cause the computer to control operation of a bioreactor vessel according to any method as aforementioned.

According to another aspect of the disclosure there is provided a device adapted to control operation of a bioprocess in a bioreactor vessel, the device comprising: means for adapting an acid supply and/or a base supply in dependence on a current pH value, a preceding pH value, and a pH setpoint. The device may further be adapted to and/or comprise means adapted to control operation of a bioprocess in a bioreactor vessel according to any method as aforementioned.

According to another aspect of the disclosure there is provided a method of controlling operation of a bioprocess in a bioreactor vessel, comprising adapting an agitation and an oxygen supply simultaneously or near-simultaneously in dependence on a dissolved oxygen setpoint and a measured dissolved oxygen. The oxygen supply may be proportional to the agitation at least in a (sub-)range of the agitation. Adapting the oxygen supply may comprise adapting a volumetric flow rate of a gas supply. Adapting the oxygen supply may comprise adapting an oxygen concentration of a gas supply. Preferably a minimum oxygen supply corresponds to a minimum agitation and a maximum oxygen supply corresponds to a maximum agitation. Preferably when an agitation increases from a minimum agitation the oxygen supply increases from the minimum oxygen supply, preferably simultaneously or near-simultaneously.

Preferably, the oxygen supply reaches maximum oxygen supply before the agitation reaches maximum agitation; more preferably wherein, when the oxygen supply reaches the maximum oxygen supply, the agitation is between 75% and 85% of the maximum agitation, yet more preferably approximately 80%. It has been observed that this may enable more precise control of dissolved oxygen in the bioreactor vessel, particularly in a late stage of the batch phase of the bioprocess, as only one parameter (i.e. agitation) is changing, so that any interactions between (increases in) oxygen supply and agitation are eliminated, which in turn may allow avoiding overshooting beyond the dissolved oxygen setpoint. Further, it has been observed that this may allow avoiding, or at least decreasing the probability of, spills as the oxygen supply (in particular, gas supply rate) does not change as much, and is fixed in the late stage of the batch phase.

Preferably, maximum oxygen supply and/or maximum agitation is reached at (or near) the end of the batch phase of the bioprocess.

Preferably, after the oxygen supply reaches the maximum oxygen supply, the agitation further increases, more preferably up to the maximum agitation, while the oxygen supply remains at the maximum oxygen supply.

According to another aspect of the disclosure there is provided a computer programme product comprising instructions which, when executed by a computer, cause the computer to control operation of a bioprocess in a bioreactor vessel, comprising adapting an agitation and an oxygen supply in dependence on a dissolved oxygen setpoint and a measured dissolved oxygen. The computer programme product may comprise instructions which, when executed by a computer, cause the computer to control operation of a bioreactor vessel according to any method as aforementioned.

According to another aspect of the disclosure there is provided a device adapted to control operation of a bioprocess in a bioreactor vessel, the device comprising: means for adapting an agitation and an oxygen supply in dependence on a dissolved oxygen setpoint and a measured dissolved oxygen. The device may further be adapted to and/or comprise means adapted to control operation of a bioprocess in a bioreactor vessel according to any method as aforementioned.

Optionally, the method produces an output.

Optionally, the method presents the output. Preferably, the method presents the output on or to a display.

Optionally, the method further comprises producing an output.

Optionally, the method further comprises presenting the output.

Optionally, the method further comprises presenting the output on or to a display.

Optionally, the method is computer-implemented.

It can be appreciated that the methods can be implemented, at least in part, using computer program code. According to another aspect of the present disclosure, there is therefore provided computer software or computer program code adapted to carry out these methods described above when processed by a computer processing means. The computer software or computer program code can be carried by computer readable medium, and in particular a non-transitory computer readable medium, that is a medium on which computer code may be stored permanently or until it is overwritten. The medium may be a physical storage medium such as a Read Only Memory (ROM) chip. Alternatively, it may be a disk, such as a Digital Video Disk (DVD-ROM), or a non-volatile memory card, e.g. a flash drive or mini/micro Secure Digital (SD) card. It could also be a signal such as an electronic signal over wires, an optical signal or a radio signal such as over a mobile telecommunication network, a terrestrial broadcast network or via a satellite or the like. The disclosure also extends to a processor running the software or code, e.g. a computer configured to carry out the methods described above.

Furthermore, features implemented in hardware may be implemented in software, and vice versa. Any reference to software and hardware features herein should be construed accordingly.

Any apparatus or device feature as described herein may also be provided as a method feature, and vice versa. As used herein, means plus function features may be expressed alternatively in terms of their corresponding structure, such as a suitably programmed processor and associated memory.

The disclosure also provides a computer program and a computer program product comprising software code adapted, when executed on a data processing apparatus, to perform any of the methods described herein, including any or all of their component steps.

The disclosure also provides a computer program and a computer program product comprising software code which, when executed on a data processing apparatus, comprises any of the apparatus features described herein.

The disclosure also provides a computer program and a computer program product having an operating system which supports a computer program for carrying out any of the methods described herein and/or for embodying any of the apparatus features described herein.

The disclosure also provides a computer readable medium having stored thereon the computer program as aforesaid.

The disclosure also provides a signal carrying the computer program as aforesaid, and a method of transmitting such a signal.

Each of the aspects above may comprise any one or more features mentioned in respect of the other aspects above.

In this specification the word ‘or’ can be interpreted in the exclusive or inclusive sense unless stated otherwise.

The disclosure extends to methods and/or apparatus substantially as herein described and/or as illustrated in the accompanying drawings.

The disclosure extends to any novel aspects or features described and/or illustrated herein. In addition, device aspects may be applied to method aspects, and vice versa. Furthermore, any, some and/or all features in one aspect can be applied to any, some and/or all features in any other aspect, in any appropriate combination.

It should also be appreciated that particular combinations of the various features described and defined in any aspects of the invention can be implemented and/or supplied and/or used independently.

As used herein, means plus function features may be expressed alternatively in terms of their corresponding structure, such as a suitably programmed processor and associated memory, for example.

Use of the words “apparatus”, “device”, “processor”, “communication interface” and so on are intended to be general rather than specific. Whilst these features of the disclosure may be implemented using an individual component, such as a computer or a central processing unit (CPU), they can equally well be implemented using other suitable components or a combination of components. For example, they could be implemented using a hard-wired circuit or circuits, e.g. an integrated circuit, using embedded software, and/or software module(s) including a function, API interface, or SDK. Further, they may be more than just a singular component.

It should be noted that the term “comprising” as used in this document means “consisting at least in part of”. So, when interpreting statements in this document that include the term “comprising”, features other than that or those prefaced by the term may also be present. Related terms such as “comprise” and “comprises” are to be interpreted in the same manner. As used herein, “(s)” following a noun means the plural and/or singular forms of the noun.

As used herein, the term “bioreactor” preferably connotes a system that supports a biologically active environment in which a chemical process is carried out by organisms or by biochemically active substances derived from organisms. This process can be aerobic or anaerobic.

As used herein, the terms “fermentation” and “bioprocess” preferably synonymously connote a chemical process carried out by organisms or biochemically active substances derived from organisms. This process can be aerobic or anaerobic.

As used herein, the term “dissolved oxygen (DO)” preferably connotes a quantity of oxygen dissolved in a liquid, preferably a dissolved oxygen concentration in a liquid. Preferably dissolved oxygen (DO) is quantified as a ratio, for example in percent, of the concentration of dissolved oxygen (DO) in the solution to a saturation concentration of oxygen in the solution. A calibration saturation concentration may be specific to a particular bioprocess, characterised by a certain temperature, pressure, and solution composition, and a person skilled in the art will be able to select a suitable calibration for a particular set of circumstances. A DO calibration procedure is preferably performed at the same conditions as the fermentation is intended to be carried out. For example, for E. coli the DO may be calibrated at: temperature 37° C., pH7, using a specific growth medium, 1 vvm (volume per volume per minute) aeration and 500 rpm agitation.

Preferred examples are now described, by way of example only, with reference to the accompanying drawings.

### BRIEF DESCRIPTION OF THE DRAWINGS

FIG. 1 is a schematic diagram of a bioreactor system according to an example of the disclosure;

FIG. 2 is a schematic diagram of a control unit forming part of the bioreactor system;

FIG. 3 is a schematic diagram of a fermentation vessel forming part of the bioreactor system;

FIG. 4 is a flow diagram illustrating an example method of controlling operation of the fermentation vessel;

FIG. 5 is a schematic diagram of an example control system used for controlling process condition(s) in the fermentation vessel;

FIG. 6a is a flow diagram illustrating an example method of controlling process condition(s) in the fermentation vessel;

FIG. 6b is a graph showing agitation rate and gassing for different bioreactor vessels;

FIG. 7 is a flow diagram illustrating example phases in a fermentation process;

FIG. 8 is a flow diagram illustrating an example method of transitioning between phases;

FIG. 9 is a flow diagram illustrating an example method of detecting a transition from a batch phase to a production phase;

FIG. 10 is a flow diagram illustrating an example method of transitioning from a batch phase to a production phase;

FIG. 11 is a flow diagram illustrating an example method of transitioning from a fed-batch phase to an end of fermentation phase;

FIG. 12 is a graph showing agitation rate and measured dissolved oxygen over the course of a bioprocess controlled by conventional cascade driven DO control;

FIG. 13 is a graph showing agitation rate and measured dissolved oxygen over the course of a bioprocess controlled by the control method illustrated in FIGS. 5-11;

FIG. 14a is a graph showing agitation rate, gas rate, measured dissolved oxygen, and measured temperature over the course of a bioprocess in a vessel with a 0.3 L working volume controlled by conventional cascade driven DO control;

FIG. 14b is a graph showing agitation rate, measured dissolved oxygen, and estimated oxygen consumption rate over the course of a bioprocess in a vessel with a 0.3 L working volume controlled by conventional cascade driven DO control;

FIG. 15a is a graph showing agitation rate, gas rate, measured dissolved oxygen, and measured temperature over the course of a bioprocess in a vessel with a 0.3 L working volume controlled by the control method illustrated in FIGS. 5-11;

FIG. 15b is a graph showing agitation rate, measured dissolved oxygen, and estimated oxygen consumption rate over the course of a bioprocess in a vessel with a 0.3 L working volume controlled by the control method illustrated in FIGS. 5-11;

FIG. 16a is a graph showing agitation rate, gas rate, measured dissolved oxygen, and measured temperature over the course of a bioprocess in a vessel with a 0.3 L working volume controlled by the control method illustrated in FIGS. 5-11;

FIG. 16b is a graph showing agitation rate, measured dissolved oxygen, and estimated oxygen consumption rate over the course of a bioprocess in a vessel with a 0.3 L working volume controlled by the control method illustrated in FIGS. 5-11;

FIG. 17 is a graph showing measured dissolved oxygen over the course of a bioprocess in a vessel with a 0.3 L working volume controlled by conventional cascade driven DO control and by the control methods illustrated in FIGS. 5-11;

FIG. 18 is a graph showing agitation rate, gas rate, measured dissolved oxygen, and measured temperature over the course of a bioprocess in a vessel with a 1 L working volume controlled by the control method illustrated in FIGS. 5-11;

FIG. 19 is a graph showing agitation rate, gas rate, measured dissolved oxygen, and measured temperature over the course of a bioprocess in a vessel with a 3 L working volume controlled by the control method illustrated in FIGS. 5-11;

FIG. 20 is a graph showing measured dissolved oxygen over the course of a bioprocess in vessels with working volumes of 0.3 L, 1 L, and 3 L, controlled by the control method illustrated in FIGS. 5-11;

FIG. 21 is a graph showing measured temperature over the course of a bioprocess in a vessel with a 0.3 L working volume controlled by conventional cascade driven DO control and by the control method illustrated in FIGS. 5-11; and

FIG. 22 is a graph showing measured pH over the course of a bioprocess in a vessel with a 0.3 L working volume controlled by the control method illustrated in FIGS. 5-11.

### DETAILED DESCRIPTION OF PREFERRED EXAMPLES

For many bioreactor processes maintaining a specific level of dissolved oxygen (DO) in the bioreactor aqueous solution is critical for optimum growth of an organism population, and for optimum effectiveness of an organism population once the population is established. The concentration of dissolved oxygen in a bioreactor follows complex behaviour, as dissolved oxygen is consumed by the organism population, which may be growing. The degree to which dissolved oxygen is supplied to the solution is dependent on a number of factors affecting transport of oxygen from air bubbles into the solution. The DO behaviour can vary significantly with temperature, media composition, agitation rate, headspace pressure, aeration rate, cell growth, cell mass, foaming and surface-active agents.

In many bioreactor processes, such as a batch bioprocess or a fed-batch bioprocess, microorganisms grow in distinct phases, and different process settings are appropriate for the different phases. In a fed-batch fermentation process a batch phase precedes a production phase (also referred to as an expression phase or a fed-batch phase). In the batch phase microorganisms are grown under batch regime for exponential population growth. Once the microorganism population has grown to the extent that nutrient limitation in the solution (e.g. a carbon source such as glucose) is imminent, the production phase is entered. During the production phase nutrients are added to feed the microorganisms and cause a desired bioprocess to be performed. During the batch phase organisms are cultivated for maximum growth of the organism population. During the production phase organisms are cultivated for maximum performance of a desired bioprocess.

While a fed-batch fermentation process can give high yields and be particularly efficient, timing of the transition from the batch phase to the production phase can be challenging, and failure to time the transition suitably can cause depletion of nutrients—typically carbon source depletion—cause and loss of performance of the fermentation process.

During the batch phase as the population growth increases exponentially the oxygen demand increases, dissolved oxygen is consumed, and controlling the process to provide a constant level of dissolved oxygen is required. An example of DO control uses a cascade of agitation, air flow, and oxygen flow: in the first step of such a cascade, an agitation rate is increased in order to maintain the desired DO level. Once a certain (e.g. maximum permitted) agitation rate is reached, the second step of the cascade is implemented, in which an air flow is increased. Once a certain (e.g. maximum permitted) air flow is reached, the third step of the cascade is implemented, in which an oxygen proportion in the gas flow is increased.

In the batch phase when the population has grown for a period under increasing oxygen consumption, the carbon source starts to become exhausted. With the carbon source becoming exhausted the oxygen uptake rate starts to decrease, eventually causing the DO to start rising again. The consequent DO spike can be detected to signal start of the production phase.

Carbon source exhaustion that causes a DO spike may also shift microorganism metabolism, and reduce peak biomass potential, so it is important to detect the onset of carbon source exhaustion accurately. While the DO spike can be readily observed, the DO controller is set to maintain the DO at a constant level, and so an effective DO controller can prevent immediate detection of a DO rise at the onset of carbon source exhaustion, but only once the controller is unable to respond fast enough to the changing oxygen uptake rate. Accurate detection of the onset of carbon source exhaustion is challenging.

It is observed that control of the bioreactor process can be improved if the transition from batch phase control to production phase control is made in dependence on an aggregate parameter, instead of on a sensed DO value alone. The aggregate parameter depends on both a sensed DO value and an aeration parameter. The aeration parameter is at least one of: an agitation rate, a gas supply rate, and a gas supply oxygen concentration. A ratio between a sensed DO value and an aeration parameter is observed to be particularly effective for accurate detection of the onset of carbon source exhaustion. The aggregate parameter can represent an estimate of an oxygen consumption rate of the bioreactor process, and can permit accurate detection of the onset of carbon source exhaustion.

Hardware Set-Up

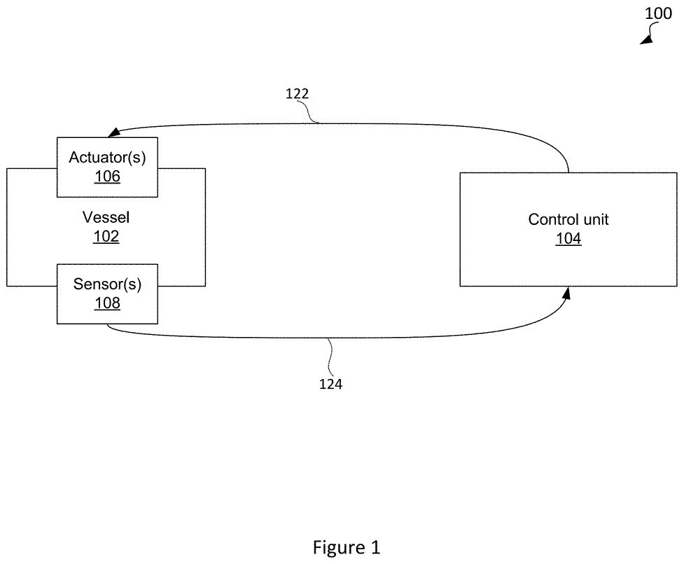

Referring to FIG. 1, according to an example, a bioreactor system 100 comprises a vessel 102 and a control unit 104. The vessel 102 can hold a liquid medium in which organisms such as microbial cells are cultivated, and it can provide an environment for a microbial cultivation process to take place. The bioreactor system 100 further comprises one or more actuator(s) 106 and one or more sensor(s) 108 associated with the vessel 102. The actuator(s) 106 affect the (process) conditions inside the vessel 102, and the sensor(s) 108 monitor the (same and/or other) conditions inside the vessel 102. The actuator(s) 106 and sensor(s) 108 may be inbuilt components of the vessel 102, or they may be separate components associated with the vessel 102. Measurements from the sensor(s) 108 are provided 124 as input(s) to the control unit 104. The control unit 104 processes the sensor input data and provides 122 control output(s) to the vessel 102. The control output(s) affect the operation of one or more of the actuator(s) 106 and thus affect the conditions inside the vessel 102, thereby completing a feedback loop.

The processing of the sensor input data by the control unit 104 may comprise comparing the sensor readings with setpoints (which may be pre-defined before fermentation commences, or determined/updated throughout the fermentation process) for the respective process conditions, and determining the required control output(s) so that the conditions are adjusted to (or closer to) the setpoints. In this way, a control system may be implemented in which the monitored vessel conditions are maintained at (or near) their setpoints. A monitored condition may not necessarily be maintained exactly at its setpoint, but rather within a range of values around the setpoint—e.g. within a percentage of the setpoint value (e.g. within ±1%, or ±5% of the setpoint value), or within a numerical range around the setpoint (e.g. assuming a 7.0 pH set point, the pH may be maintained within 7.0±0.3). The range may be symmetrical (as in the examples provided) or asymmetrical around the setpoint.

In the present example, the actuator(s) 106 and sensor(s) 108 and the control unit 104 are connected via physical electrical connections which allows them to communicate 122, 124 as described above. Alternatively, the actuator(s) 106 and sensor(s) 108 and the control unit 104 may communicate via connections established via the Internet, in which case the actuator(s) 106 and sensor(s) 108 and the control unit 104 may be arranged to communicate with the Internet e.g. via wired Ethernet connections and access points, and/or via a cellular radio network link using an appropriate communication standard, such as Global System for Mobile Communications (GSM), Universal Mobile Telecommunications System (UMTS) or Long-Term Evolution (LTE). The actuator(s) 106 and sensor(s) 108 and the control unit 104 may also be arranged to communicate with each other (and/or with an access point) via another short-range wireless communication connection, such as: a Wi-Fi® connection, a Bluetooth® connection, IR wireless connection, ZigBee® connection, or some other similar connection. Any of the above described possible connections between the control unit 104 and the actuator(s) 106 and sensor(s) 108 may be used in combination—e.g. to provide one or more back-up connections in case the primary connection fails; and/or to share/spread the sent data, for example to decrease the used bandwidth in each connection.

Example parameters (or process conditions) measured by the sensor(s) 108 and provided 124 as measurement values to the control unit 104 are listed in Table 1 below. The sensor(s) 108 used to obtain the parameter measurements may be any conventional sensors. For example, a transducer (such as a pH electrode) may be used to measure the pH, or infrared (IR) or Raman spectroscopy may be used to measure the concentration of one or more compounds (e.g. carbon dioxide or methane) in the medium. Some of the parameters listed in Table 1 may be measured indirectly and determined based on the values of other measured parameter(s)—e.g. the oxygen consumption may be estimated using a ratio of the agitation speed to the dissolved oxygen concentration (DO) process value. The parameters listed in Table 1, as well as further not listed parameters, may be measured through a range of methods, such as optical chemical, electrochemical, radiation, radar, acoustics, vision, viscosity, strain, spectrometry, gas and/or liquid chromatography, tomography, thermal and/or electrical conductivity, among others. The sensor(s) 108 may be for directly contacting a liquid medium in the vessel 102 and/or a headspace gas. The sensor(s) 108 may be arranged inside or outside of the vessel 102. The sensor(s) 108 may provide contactless sensing of the bioreactor. The sensors 108 may be real-time sensors, delayed sensors (e.g. sensors that require a sample to be removed from the medium), or a combination thereof (e.g. a subset of the sensors being real-time, and a subset delayed). In the present example, the control system relies primarily on real-time (or near-real-time) sensors which monitor and provide measurements of the process conditions in (or in nearly) real-time, with only a minor (e.g. less than 1 second) delay (lag time).

TABLE 1

| | Examples |
|---|---|
| Measured | pH; |
| parameters | temperature; |
| | dissolved oxygen concentration (DO); |
| | oxygen uptake rate (OUR); |
| | oxygen transfer (transmission) rate (OTR); |
| | oxygen consumption; |
| | substrate and/or biomass and/or inducer and/or feed |
| | concentration; |
| | glucose uptake rate; |
| | carbon dioxide production rate; |
| | pressure; |
| | agitation speed (e.g. in revolutions per minute (rpm)); |
| | conductivity; |
| | turbidity; |
| | pump speed (e.g. for the base and/or feed and/or induction |
| | pump(s)); |
| | supply volumetric flow rate (e.g. feed supply volumetric flow |
| | rate); |
| | gas (volumetric) flow rate (e.g. for an air supply, or for a |
| | concentrated oxygen supply); |
| | oxygen concentration in gas supply; |
| | suspended solids; |
| | oxidation-reduction potential; |
| | ozone; |
| | chlorine |

The actuator(s) 106 affect the (process) conditions inside the vessel 102. For example, the actuators 106 may vary the temperature in the vessel via a heating system (e.g. a thermal jacket), supply various materials to the vessel (e.g. air to provide oxygen to the liquid medium, or a basic (or acid) solution to adjust the pH of the liquid medium), or act to improve mixing within the vessel (e.g. via a stirer powered by a motor). These and other examples of actuators are listed in Table 2 below. Further actuator(s) may be associated with the vessel 102 to affect (and thus enable control of) any other process conditions in the vessel 102.

TABLE 2

| | Examples |
|---|---|
| Actuators | Heating/cooling system (e.g. thermal jacket); |
| | agitation system (e.g. comprising a stirrer); |
| | acid and/or base supply (e.g. via a pump; e.g. a peristaltic |
| | pump); |
| | resource (e.g. air/oxygen) supply (e.g. a compressed air |
| | supply); |
| | feed (medium) supply; |
| | inducer supply; |
| | medium supply; |
| | antifoam supply |

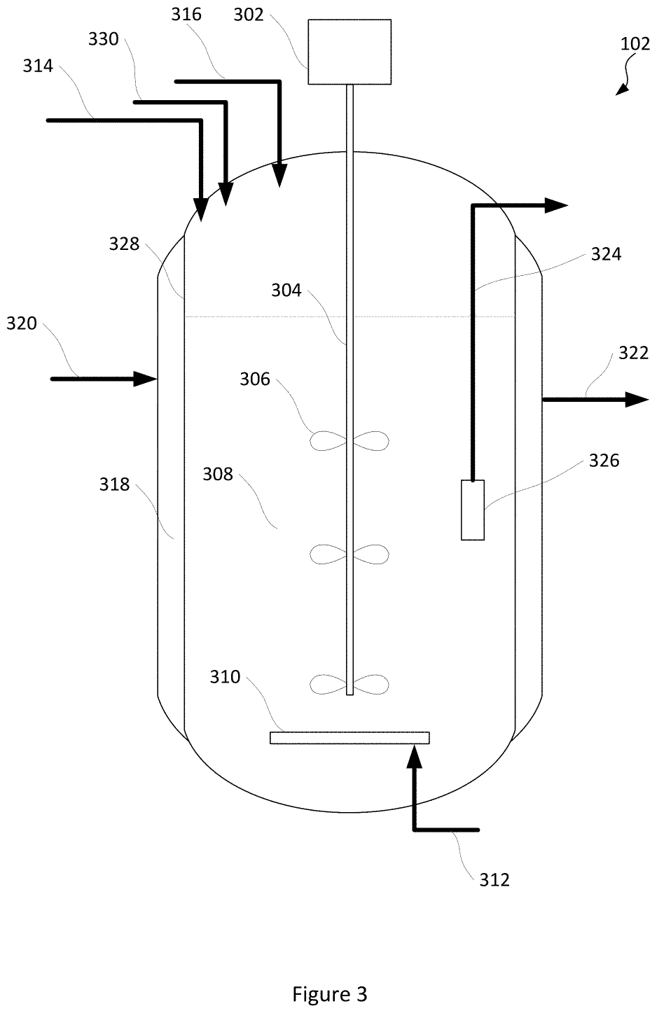

An example bioreactor comprising a number of specific sensors that measure a subset of the parameters listed in Table 1 and comprising a subset of the actuators listed in Table 2 is described with reference to FIG. 3 below. A person skilled in the art would appreciate that any other combination of sensors (measuring parameters listed in Table 1 and/or other parameters) or actuators (listed in Table 2 and/or others) could be provided in the bioreactor system 100, without departure from the scope of the claims.

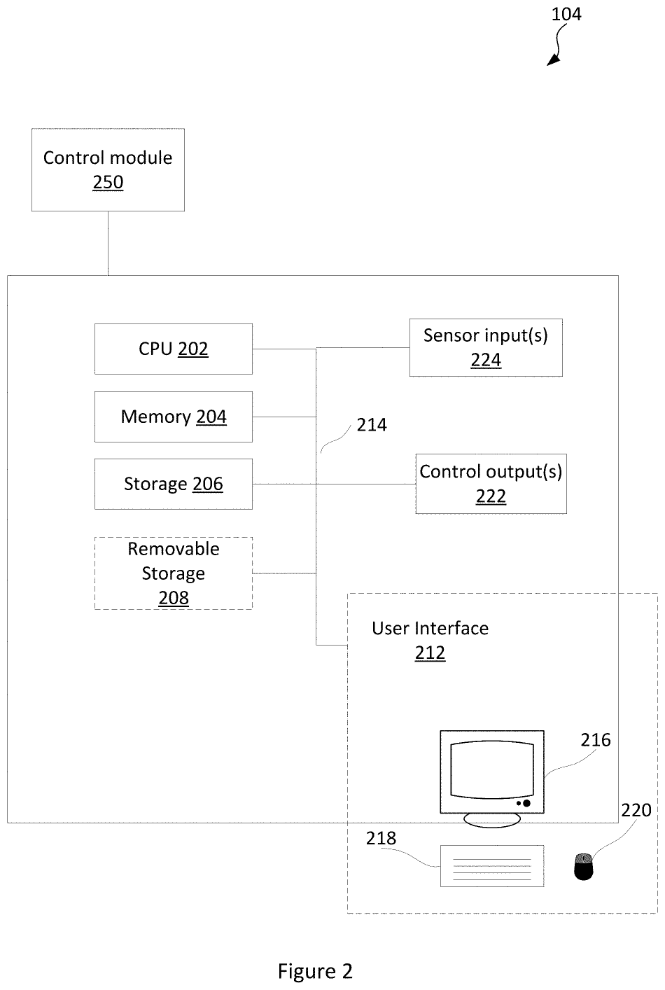

Referring to FIG. 2, a control unit 104 is a computer device which comprises a Central Processing Unit (CPU) 202, memory 204, storage 206, sensor input(s) 224, and control output(s) 222, coupled to one another by a bus 214. The control unit 104 may further comprise a removable storage 208, and a user interface 212, likewise coupled to one another and to the above-described control unit components by a bus 214.

As mentioned above, the control unit 104 processes the sensor input data and provides 122 control output(s) to the actuators 106 associated with the vessel 102. In other words, the control unit 104 determines the control signals for the actuator(s) 106 at the vessel 102 based on the process conditions measured by sensor(s) at the vessel 102. A control module 250 may be installed on the control unit 104 to perform this task. The control module is a software application responsible for processing the data from the sensor input(s) 224 (and optionally data input via the user interface 212), and for determining the control output(s) 222 used for controlling the actuator(s) 106 for the vessel 102. The control module 250 has associated instructions that are also in the form of computer executable code, stored in the memory 204, the storage 206 and/or removable storage 208. The inner workings of the control module 250 are described in further detail in the sections below.

The control module 250 may further optionally be used for user input for controlling the actuator(s) 106 in the vessel 102, in particular if a user interface 212 is present. The control module 250 may comprise a user interface (UI) module that provides an interface for operation of the control unit 104 by a user. The control module 250 may also comprise an analytics module which can be used for performing various data analytics on data related to the bioreactor system 100 (e.g. process conditions measurements provided 124 to the control unit 104).

The sensor input(s) 224 comprise means for receiving the process condition measurement(s) from sensor(s) 108 typically provided in the vessel 102, and the control output(s) comprise means for providing control signal(s) to actuator(s) 106 typically provided in the vessel 102. In the present example, these means are input/output ports (or sockets or connectors) for the physical electrical connections between the control unit 104 and the sensor(s) 108 and actuator(s) 106, such as twisted-pair connectors, coaxial cable connectors or fibre-optic connectors. A person skilled in art would appreciate that many alternative means are available (including wireless means) and could be used without departure from the scope of the claims.

The user interface 212 comprises and an input/output device which in this example is a display 216, a keyboard 218 and a mouse 220. In other examples, the input/output device comprises a touchscreen such as a Thin-Film-Transistor (TFT) Liquid Crystal Display (LCD) display or an Organic Light Emitting Diode (OLED) display, or other appropriate display. The user interface is arranged to provide indications to the user (e.g. of the current process conditions in the vessel 102), under the control of the CPU 202, and to optionally receive inputs from the user, and to convey these inputs to the CPU 202 via the communications bus 214. The user interface 212 may thus be used to perform manual adaptation of the control of the bioreactor system 100, for example in order to specify desired setpoints or control parameters or to calibrate sensors or actuators or to override an automated control. This may provide a useful fail-safe mechanism, e.g. by allowing manual override of the automated control if a certain measured process condition (e.g. temperature) falls outside a defined range.

The CPU 202 is a computer processor, e.g. a microprocessor. It is arranged to execute instructions in the form of computer executable code, including instructions stored in the memory 204, the storage 206 and/or removable storage 208. The instructions executed by the CPU 202 include instructions for coordinating operation of the other components of the control unit 104, such as instructions for controlling the control output(s) 222 as well as other features of a control unit 104 such as a user interface 212 and/or audio system (not shown).

The memory 204 stores instructions and other information for use by the CPU 202. The memory 204 is the main memory of the control unit 104. It usually comprises both Random Access Memory (RAM) and Read Only Memory (ROM). The memory 204 is arranged to store the instructions processed by the CPU 202, in the form of computer executable code. Typically, only selected elements of the computer executable code are stored by the memory 204 at any one time, which selected elements define the instructions essential to the operations of the control unit 104 being carried out at the particular time. In other words, the computer executable code is stored transiently in the memory 204 whilst some particular process is handled by the CPU 202.

The storage 206 provides mass storage for the control unit 104. In different implementations, the storage 206 is an integral storage device in the form of a hard disk device, a flash memory or some other similar solid-state memory device, or an array of such devices. The storage 206 stores computer executable code defining the instructions processed by the CPU 202. The storage 206 stores the computer executable code permanently or semi-permanently, e.g. until overwritten. That is, the computer executable code is stored in the storage 206 non-transiently. Typically, the computer executable code stored by the storage 206 relates to instructions fundamental to the operation of the CPU 202, the various inputs and outputs (e.g. user interface 212, control output(s) 222, sensor input(s) 224) and other installed applications or software modules (such as the control module 250).

The removable storage 208 provides auxiliary storage for the control unit 104. In different implementations, the removable storage 208 is a storage medium for a removable storage device, such as an optical disk, for example a Digital Versatile Disk (DVD), a portable flash drive or some other similar portable solid-state memory device, or an array of such devices. In other examples, the removable storage 208 is remote from the control unit 104 and comprises a network storage device or a cloud-based storage device.

As mentioned, the control system is managed via a control-unit-based software (control module 250) which is implemented as a computer program product, which is stored, at different stages, in the memory 204, storage device 206, and/or removable storage 208. The storage of the computer program product is non-transitory, except when instructions included in the computer program product are being executed by the CPU 202, in which case the instructions are sometimes stored temporarily in the CPU 202, or memory 204. It should also be noted that the removable storage 208 is removable from the control unit 104, such that the computer program product may be held separately from the control unit 104 from time to time.

In an alternative example, the control unit 104 may further comprise an Internet communications module configured to establish connection(s) with the Internet. The Internet communications module may typically comprise an Ethernet network adaptor coupling the bus 214 to an Ethernet socket. The Ethernet socket may be coupled to a network.

Referring to FIG. 3, an example bioreactor is shown. In the present example, the bioreactor is a stirred-tank fermentation vessel 328. The fermentation vessel 328 holds a (liquid) medium 308, sensor probes 326, and various actuators (e.g. 314, 316, 318, 330, 312). The fermentation vessel 328 is a single-use vessel, though it could alternatively be a re-usable vessel. An advantage of a single-use vessel is that it removes the need to clean the vessel after use and so may allow avoiding fouling which may otherwise reduce the overall efficiency of the fermentation vessel.

The fermentation vessel 328 is of polystyrene and polycarbonate and has a working (inner) volume between 50 mL and 50 L, but suitable vessels may be fabricated in a range of different materials and be of many different sizes. For example, the vessel may be formed of a polymer or a glass or a steel (notably a stainless steel), or it may be formed of a combination of materials such as a polymer blend or combination, a glass-lined steel, or a polymer-lined glass. The vessel may be formed of a disposable liner and a rigid support. The vessel size may vary from less than 1 L to more than 10,000 L.

The actuators comprise: a heating/cooling system; an agitation system; and an oxygen supply system. The actuators may comprise further supply systems such as: a feed supply, an acid/base supply, and/or an inducer supply.

The heating/cooling system is used to maintain the fermentation vessel contents at (or near) a desired temperature (which may vary throughout the fermentation process) so as to improve the efficiency of the fermentation process. The heating/cooling system can be used to maintain a given temperature despite the reactions within the fermentation vessel 328 typically being exo- or endothermic, and the agitation system generating heat by agitating the liquid medium. In the present example, the heating/cooling system is a thermal jacket 318. The thermal jacket may be heated or cooled in a range of different ways, with or without a heat transfer fluid. For example, the thermal jacket may be heated/cooled using a heat transfer fluid entering the jacket 318 via inlet 320 and exiting via outlet 322 to add or remove heat, or using a thermoelectric liquid-free cooling system (not shown) based on the Peltier effect (wherein a DC electric current flowing through a device transfers heat from one side to the other). A heat transfer fluid based thermal jacket may for example be: a single external jacket—a single chamber jacket surrounding the fermentation vessel 328, a half coil jacket—a half pipe attached to (e.g. welded around) the fermentation vessel 328 to create a semi-circular flow channel, or a constant flux jacket which comprises a plurality of smaller jacket elements of which only a subset may be used to heat/cool the fermentation vessel at a time.

The agitation system is used to mix the contents of the fermentation vessel 328 so as to maintain the cells in a homogeneous condition and improve the transport of nutrients and/or oxygen to the desired product(s). The agitation system allows mixing which in turn acts to eliminate (or reduce) gradients in the fermentation vessel 328—e.g. gradients of concentration (cell, medium, feed, inducer, oxygen). In the present example, the agitation system comprises a motor (drive unit) 302 that drives an agitator shaft 304, and one or more impeller blades 306 mounted on the agitator shaft 304. The impeller blades may be of any design, such as Rushton, paddle, marine, or pitched. Optionally, the agitator system may further comprise baffles (not shown). The baffles comprise one or more stationary blades that break up flow(s) caused by the rotating agitator and thus improve mixing and mass transfer within the fermentation vessel 328.

For aerobic (and some anaerobic) fermentation processes, oxygen must be supplied to the vessel. Since oxygen is relatively insoluble in the liquid medium 308 (typically largely comprised of water), oxygen is often added to the fermentation vessel 328 continuously. In the present example, the oxygen supply system provides a continuous supply of oxygen to the fermentation vessel 328. The oxygen supply system comprises an aerator 310 that is supplied with air (and/or purified oxygen) via inlet 312.

The fermentation vessel may further comprise a feed supply 314, an acid/base supply 316, and an inducer supply 330 arranged to supply materials into the fermentation vessel 328. In the present example, the acid/base supply is a base supply and supplies a basic solution to adjust the pH of the medium 308. Alternatively or additionally, the acid/base supply may supply an acidic solution. The feed supply supplies the nutrient(s) required by the organisms or cells undergoing the fermentation process in the fermentation vessel 328. In the present example, the feed supply supplies a carbon source—e.g. sugar cane juice, glucose, sucrose, glycerol, or any other suitable carbon source (the candidate options largely depending on the output product). The inducer supply supplies an inducer/catalyst to improve and/or facilitate chemical and/or biological processes in the production phase. For example, an inducer may improve and/or facilitate transcription and gene expression to take place so that the target protein may be more efficiently produced in the production phase of the fermentation process. These supplies typically supply material in a liquid form (e.g. aqueous solutions), and may include pumps as actuators, for example in the form of peristaltic pumps. In the illustrated example the acid/base supply includes a base pump (referred to as pump B below), the feed supply includes a feed pump (referred to as pump A below), and the inducer supply includes an inducer pump (referred to as pump C below).

A number of sensors 326 monitor and measure process conditions within the fermentation vessel 328 and/or conditions related to the fermentation vessel 102, and provide the measurements to the control unit 104 via electrical connection(s) 324. The sensor 326 in the illustrated example comprise: a temperature sensor, a pH sensor, an agitation speed sensor, one or more gas flow rate sensor (e.g. to measure the flow rate of air supplied to the fermentation vessel 328—e.g. the flow rate at inlet 312), a dissolved oxygen concentration (DO) sensor, an oxygen concentration sensor (used to measure the concentration of oxygen in the air supply), and optionally one or more flow rate (e.g. pump speed) sensors that measure the volume per unit time of materials supplied to (e.g. pumped into) the fermentation vessel 328 (e.g. materials supplied by the feed supply 314, acid/base supply 316, and inducer supply 330 by way of actuator pumps as described above). The sensors 326 may further comprise a working volume sensor that measures the working volume in the fermentation vessel (e.g. by measuring the level of the liquid medium 308 in the fermentation vessel 328).

Generally a vessel 102 for a bioreactor may comprise further additional features, or indeed fewer features than described above. Possible additional features include: further inlets or outlets (e.g. pumps)—for example an outlet for an effluent, or for exhaust/waste gases; further supplies such as antifoam supply; and/or further sensor(s) or actuator(s) as described with reference to Tables 1 and 2 above.

The present disclosure could be implemented using numerous off-the-shelf bioreactor systems using autoclavable vessels or single-use vessels, such as the Eppendorf® DASbox® Mini Bioreactor System, using Eppendorf® BioBlu® (0.3f, 1f, or 3f) Single-use Vessels, and/or the Eppendorf® DASGIP®, Eppendorf® BioFlo® 120/320 bioreactor systems, using Eppendorf® BioBLU® Single-Use Vessels. The vessel 102, actuator(s) 106, sensor(s) 108 and control unit 104 may include any of the features of such conventionally known bioreactor systems.

The vessel 102, actuator(s) 106, sensor(s) 108 and control unit 104 may be integrated together to various degrees. It should also be appreciated that a number of alternative bioreactor system designs would be known to a person skilled in the art, any such alternative bioreactor system could be used to implement the below described methods.

In an alternative example, multiple fermentation vessels are provided for (and/or connected to) a control unit.

In a further alternative example, multiple control units are connected to each fermentation vessel. For example, each control unit may be responsible for controlling one process condition (e.g. the pH) within the fermentation vessel; or, multiple control units may each perform a subset of the processing needed to determine control signal(s) for the fermentation vessel—e.g. one control unit may pre-process the input sensor data, and another control unit may then determine the control signal(s).

Control Module

The control module 250 is responsible for processing the data from the sensor input(s) 224 (and optionally data input via the user interface 212), and for determining the control output(s)/signal(s) 222 used for controlling the actuator(s) in the fermentation vessel 102. In particular, the control module 250 may be used to implement automated control of the process conditions in the fermentation vessel—within a given phase of the fermentation process (e.g. batch phase) and/or across and/or between multiple fermentation phases. In particular, the control module 250 is responsible for controlling the transitioning between a batch phase and a production phase.

In the present example, the primary functions of the control module 250 are to:
Maintain controlled variable(s) (process condition(s) to be controlled) at (or near) their respective setpoints. Example controlled variables include pH, temperature and/or dissolved oxygen concentration (DO) within the fermentation vessel 102.
Determine the (preferably current) fermentation phase—in particular, detect a transition from a batch phase to a production phase. Notably, the controlled variable setpoints may depend on the determined fermentation phase.
Manage the transition between fermentation phases (in particular between a batch phase and a production phase). This may include supplying materials (e.g. feed and/or inducer) to the fermentation vessel 328; and/or implementing a gradual (e.g. staggered, linear, or otherwise) change between controlled variable setpoints in different fermentation phases.

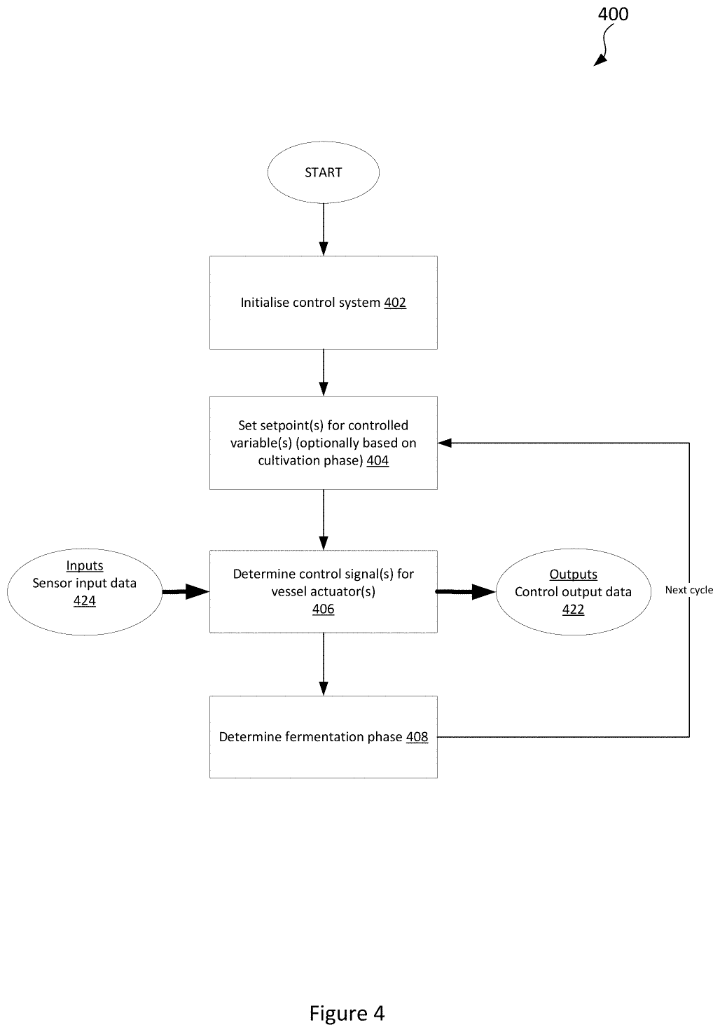

Referring to FIG. 4, an example method 400 of controlling operation of the fermentation vessel 102 is shown.

First, the control system is initialised 402. This step may include setting the parameters for constant variables such as: fermentation vessel 102 dimensions, properties of the actuator(s) in the fermentation vessel (e.g. minimum/maximum supply (pump) speed, minimum/maximum agitation (stirring) speed, minimum/maximum gas (e.g. air) flow rate, etc.); variables related to fermentation process (e.g. the starting phase, phase duration(s), or starting process condition(s) setpoint(s)); feed and/or inducer properties, etc.); and/or control system related variables (e.g. sample time (interval between cycles), which defines how frequently steps 404-408 of method 400 are repeated).

Next, the setpoint(s) are set 404 for the process condition(s) to be controlled (in other words, for the controlled variables). The setpoint(s) are preferably re-set at each cycle of method 400 as the setpoint(s) may depend on the fermentation phase. In the present example, the process conditions which are controlled—i.e. the process conditions for which control signal(s) are determined for corresponding actuator(s) so as the process conditions are kept at (or near) their setpoint(s)—are at least dissolved oxygen concentration (DO), pH, and temperature. Preferably, the process conditions refer to process conditions within the fermentation vessel 328.

Subsequently, based on sensor input data 424 (which may be pre-processed), and the setpoint(s) set at step 404, the control module 250 determines 406 control signal(s) for the fermentation vessel actuator(s) and outputs control output data 422 which is transmitted to the fermentation vessel actuator(s) as described above. The determination of the control signal(s) may further be based on the fermentation phase—e.g. some actuator(s) (e.g. inducer supply) are controlled based on the fermentation phase instead of, or in addition to, being based on the process conditions setpoints. Step 406 is described in further detail in the sections below.

Next, the control module 250 determines 408 the fermentation phase. Preferably, this determination corresponds to the fermentation phase in the present (current) cycle. As mentioned above, the setpoint(s) and/or the control signal(s) determined in steps 404 and 406 respectively may depend on the fermentation phase. Further details of the fermentation phases and how they may be determined are provided in the sections below.

Preferably, method 400 is performed in real-time (or in near real-time) so that the control module 250 may quickly react to changing process conditions in the fermentation vessel 102.

Preferably, the time interval between cycles is between 500 ms and 1 minute, yet more preferably it is 10 seconds. A too short interval may lead to “overcontrol” in the sense that the control signal(s) may be needlessly modified before they have had the time to affect the controlled variable(s); whereas a too long interval may introduce an excessively large lag in the system causing it to be slow to react to changing conditions within the fermentation vessel 328 (e.g. detecting a transition between phases late or not at all). The above-described range was chosen to balance this trade-off.

It should be appreciated that the order of steps 404-408 could be interchanged in any combination. As shown in FIG. 4, the control signal(s) are determined 406 based on the setpoint(s) set 404 in the current cycle, and optionally the fermentation phase determined 408 in the previous cycle; however the control signal(s) could be set based on the setpoint(s) set in the previous (or current) cycle and optionally on the fermentation phase determined in the current (or previous) cycle.

It should also be appreciated that the distinctions between steps 402-408 are arbitrary and were simply included for clarity. The actions within these steps could in fact be split into more (whereby each of steps 402-408 may be considered a method of its own) or fewer steps.

Process Condition(s) Control System

In order to maintain controlled variable(s) at (or near) their respective setpoint(s), the control module 250 implements one or more control loops (or controllers). The control loop(s) may comprise single-input single-output (SISO) control loop(s); and/or multiple-input multiple-output (MIMO) control loop(s). The controller(s) may be non-adaptive (using fixed setpoint(s)), or adaptive (whereby a controller adjusts setpoint(s) based on process condition(s) in which case it may be based on a mathematical model of the fermentation process or be a model-free adaptive controller (using a dynamic feedback system rather than a model). Furthermore, each controller may determine control signal(s) for the actuator(s) in a deterministic manner (e.g. based on a given model of the system and/or interactions between input(s) and output(s)) or in an empirical manner (e.g. iteratively modifying the control signal(s) based on observed process condition(s) until the controlled variable is at its setpoint).

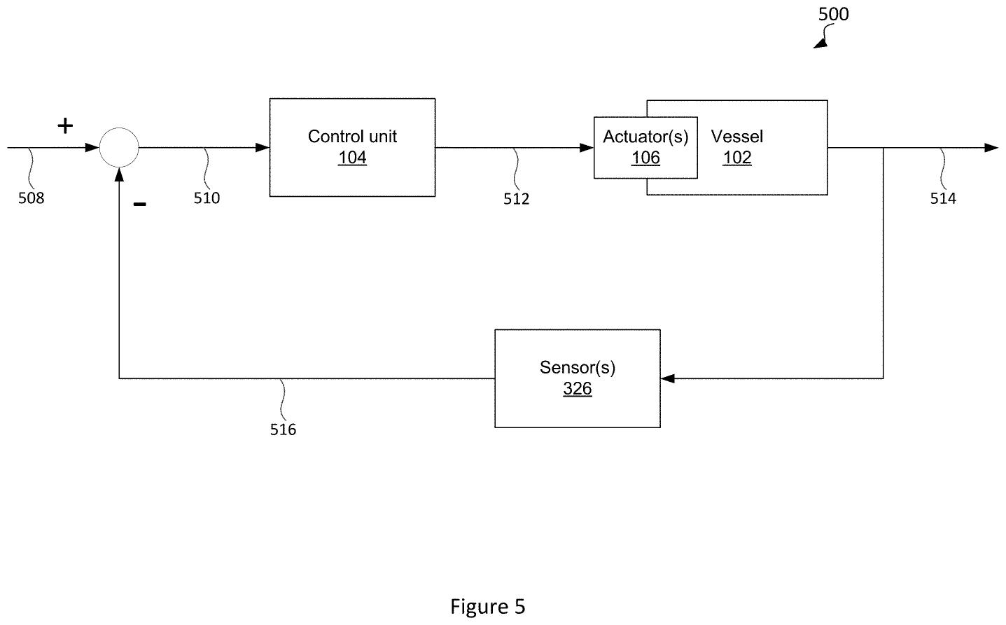

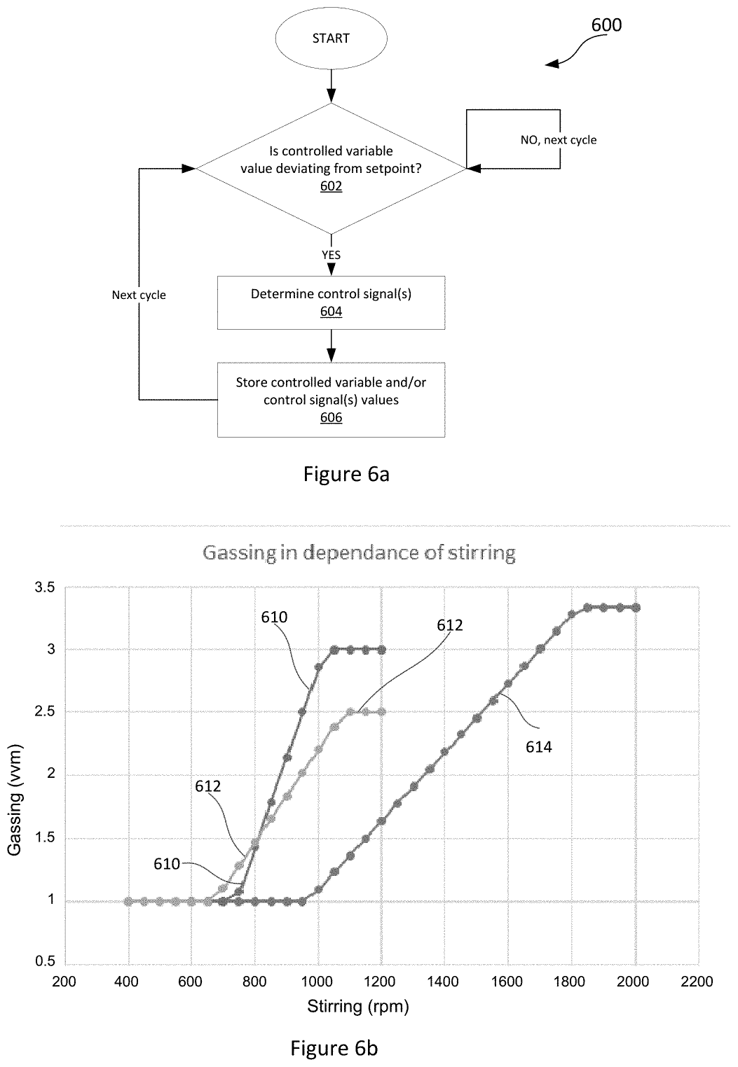

Referring to FIGS. 5 and 6a, an example implementation 600 of method 406, and an example control system 500 are shown. FIGS. 5 and 6a correspond to a single-input controller 504 with a single controlled variable, and one or more outputs (control signal(s) 512). Preferably, method 600 is repeated periodically (in cycles), as shown in FIG. 6a. FIG. includes a number of features identical to those described with reference to FIGS. 1 to 4, and corresponding reference numerals are used to refer to those features.

Referring to FIG. 5, an example control system used for controlling process condition(s) in the vessel 102 is shown. The control system 500 determines the difference (or error) 510 between a controlled variable setpoint 508 and a measured controlled variable value 516 based on measurements of the controlled variable's value 514 by sensor(s) 108. This error 510 is then passed as an input to a controller within the control unit 104 which implements a controller function and determines the actuator/control signal(s) 512 for the actuator 106. The actuator(s) 106 affect the controlled variable in the vessel 102 so as to bring it in line with the setpoint 508. Using pH as an example controlled variable, the setpoint 508 may represent the desired pH (typically 7.0), the actuator may be the acid/base supply 316, and the sensor 326 may be a pH electrode.

FIG. 6a illustrates an example method of controlling process condition(s) in the vessel. The control module 250 first determines 602 the error 510 between the measured controlled variable value 514 and the setpoint 508.

If the error 510 is below a pre-defined threshold—e.g. it is zero or within a defined range around the setpoint (e.g. within ±1% or 5% of the setpoint value; or greater/lesser than zero), method 600 terminates and the control signal(s) are kept at their previous values (e.g. as determined in the previous cycle). Method 600 is then repeated in the next cycle, starting at step 602.

If the error 510 exceeds the pre-defined threshold, the control module 250 determines the controlled signal(s) 512 based on a controller function. Example controller functions for the control of the pH and DO (for pH and DO controlled variables) are described in further detail below. The control module 250 may then store 608 the controlled variable and/or control signal(s) values so that they may be used as inputs for step 604 in the next cycle(s). Method 600 is then repeated in the next cycle, starting at step 602.

The frequency at which control signal(s) are adjusted to control a given process condition may vary across conditions. For example, control signal(s) may be adjusted less frequently for pH and DO, than for temperature.

Example pH Controller Function

In the present example, the pH within the fermentation vessel 328 is controlled using the acid/base supply 316 (e.g. pump B) which serves as the actuator described with reference to FIGS. 5 and 6a. The flow rate (or pump speed) of the base supply (which supplies an acidic and/or a basic solution with the aim of increasing or decreasing the pH) may be determined in an empirical manner, whereby the flow rate is set in dependence on the pH measured in one (preferably the current) cycle (pH₁), the setpoint pH (pH_{SP}), and the pH measured in a preceding (preferably the previous) cycle (pH₀). In more detail, the algorithm for determining the acid/base (in this case, base only) supply flow rate may be as follows:

| [symbol] | Load value(s) from preceding cycle: counter_{pH} (counting the number of consecutive cycles during which the condition | |
|---|---|---|
| | (pH₁ / pH₀) ≤ 1 is met); pH₀ | |
| [symbol] | If pH₁ < pH_{SP} | |
| | ◯ | If (pH₁ / pH₀) ≤ 1 |

| | |
|---|---|
| ▪ | Increment counter_{pH} |
| ▪ | Set pump B speed as (k × (pH_{SP} − pH₁) × c^{counter}^{_{pH}}), wherein k and c are arbitrary constants |

chosen such that the pump speed may be increased gradually in such a manner that the setpoint is
reached in few cycles while simultaneously avoiding overshooting the setpoint. k may be
proportional to the maximum pump B speed. Example k and c values may be 0.35*(max pump B
speed) and 1.025 respectively.

| | |
|---|---|
| ◯ | Else |

| | |
|---|---|
| ▪ | Reset counter_{pH} (set counter_{pH} to zero) |
| ▪ | Maintain previous pump B speed |

| | |
|---|---|
| ◯ | Store pH₁ and counter_{pH} for future cycle(s) (e.g. pH₁ may be used as pH₀). (This step being independent of the |
| | if/else condition (pH₁ / pH₀) ≤ 1.) |

| | |
|---|---|
| [symbol] | Else - switch off pump B (set pump B speed to zero); and reset counter_{pH}. |

The above example algorithm was described with reference to a base supply (supplying a basic solution only), which may be relevant to a fermentation process in which the liquid medium 308 pH decreases in the absence of an acid/base supply. A person skilled in the art would appreciate that this description could easily be extended to an acid supply (supplying an acidic solution only) or to an acid/base supply (supplying both an acidic and a base solution).

Example DO Controller Function

In the present example, the DO level within the fermentation vessel 328 is controlled using the agitation system and/or the oxygen supply system which serve as the actuator(s) described with reference to FIGS. 5 and 6a. The stirring speed (N) of the agitation system, and/or the oxygen concentration (e.g. air vs. O₂ enrichment) and/or the volumetric flow rate of the oxygen supply system are controlled via control signal(s) 512 so as to maintain the DO at (or near) its setpoint.

As mentioned, the agitation system allows mixing which in turn acts to eliminate (or reduce) gradients in the fermentation vessel 328—e.g. gradients of oxygen (in liquid/dissolved and/or gas form) concentration. Further, agitation disperses oxygen bubbles (e.g. provided by an aerator 310) and promotes mass transfer of the gas bubbles through the gas-liquid (medium) interface. Agitation thus improves the oxygen transfer rate (OTR) from gas to liquid form. Therefore, increasing agitation generally can increase the DO level. The oxygen supply system may likewise affect the OTR—increasing oxygen supply to the fermentation vessel 328 increases oxygen availability which in turn may increase the OTR and the DO level/concentration.

The DO level may be controlled by adjusting the stirring speed in one (preferably the current cycle) (N₁) based on: the stirring speed in a preceding (preferably the previous) cycle (N₀), the DO setpoint (DOₛₑₜ), the DO value in one cycle (DO₁), and preferably the DO value in a/the preceding (preferably the previous) cycle (DO₀). In more detail, example formulae for setting N₁ in a deterministic manner are shown as equations (1), (2a) and (2b) as follows:

$${N}_{1} = {N}_{0} \times ( 1 + \frac{D {O}_{S P} - D {O}_{1}}{D {O}_{S P} \times τ} ) \qquad ( 1 )$$

$${N}_{1} = {N}_{0} \times ( 1 + \frac{( D {O}_{S P} - D {O}_{1} ) - ( D {O}_{1} - D {O}_{0} )}{D {O}_{S P} \times τ} ) \qquad ( 2 a )$$

$${N}_{1} = {N}_{0} + \frac{{N}_{max}}{{V}_{B R}} \cdot ( ( {DO}_{S P} - {DO}_{1} ) - ( {DO}_{1} - {DO}_{0} ) ) \qquad ( 2 b )$$

wherein τ denotes the time interval between the one cycle and the preceding cycle as described above (preferably the sampling time or time in-between cycles), and where

$$\frac{{N}_{max}}{{V}_{B R}}$$

is a scaling factor that characterises a relative mixing power (with Nₘₐₓ—maximal agitation speed and V_{BR}—working volume of bioreactor).

Equation (2a) takes into account the rate of change of DO

$$( \frac{dDO}{d τ} )$$

which is approximated as

$$( \frac{dDO}{d τ} ∼ \frac{D {O}_{1} - D {O}_{0}}{d τ} ∼ \frac{D {O}_{1} - D {O}_{0}}{τ} ) ,$$

assuming a small τ; equation (2b) takes into account the rate of change by way of the expression (DO₁−DO₀). Equation (2a) can also be written as:

$${N}_{1} = {N}_{0} \times ( 1 + \frac{( D {O}_{S P} - D {O}_{1} ) - \frac{dDo}{d τ}}{D {O}_{S P} \times τ} ) .$$

Compared to equation (1), equations (2a) and (2b) reduce or increase the change depending on the foregoing DO value. This can have a dampening or anticipatory effect, in particular when approaching the setpoint. The more rapidly the DO value has recently changed, the greater the controlling or dampening effect. Less fluctuation about a setpoint can be enabled with such control, and the DO value can be controlled more smoothly, providing more even conditions for a bioprocess.

Compared to equation (2a), equation (2b) takes into account relative mixing power of a given vessel. The scaling factor

$$\frac{{N}_{max}}{{V}_{B R}}$$

scales the agitation change to the size of the vessel. In particular for a larger vessel this can prevent overshooting. The mixing process m vessels of different sizes can be characterised by the mixing power per volume (W/L). The delivered mixing power relates to the physical dimensions of the stirrer cubically, i.e. power˜linear_size³. Larger vessels typically use larger size stirrers and often show more efficient power delivery. For example, mixing at 1200 rpms in a 3 L bioreactor is comparable to mixing at 2000 rpm in a 0.3 L bioreactor. In another example, increasing N₀=1000 rpm by 20 rpms in a 0.3 L bioreactor is expected have a similar effect on DO as increasing N₀=800 rpm by 3 rpms in a 1 L bioreactor.

Optionally, the scaling factor in equation (2b) may instead be

$$\frac{{N}_{max} - {N}_{min}}{{V}_{B R}} .$$

Accordingly, an example algorithm for determining the agitation system stirring rate/speed (N₁) so as to maintain the DO at DO_{SP}, based on equation (1) or equation (2a) or equation (2b), may be:

| [symbol] | Load value(s) from preceding cycle: DO₀ | |
|---|---|---|
| [symbol] | If (DO₁ > (i * DO_{SP})) or (DO₁ < (j * DO_{SP})); wherein i and j are arbitrary constants that define the desirable range of | |
| | DO values around the setpoint, e.g. i=1.02; j=0.98. | |
| | ◯ | Determine N₁ control signal based on equation (2a) (or alternatively based on equation (1) or equation (2b)) |
| | ◯ | Optionally, prior to setting the control signal to N₁, the controller function may include checking whether N₁ |
| | | falls within a permitted range (e.g. not below the minimum permitted value (Nₘᵢₙ) and not above the maximum |
| | | permitted value (Nₘₐₓ) for the given agitation system and/or fermentation vessel and/or fermentation process). |
| | | For example, there may be an upper bound to the agitation speed (mixing) so as to prevent excessive shear |
| | | forces in the medium that may lead to cell death. |
| | ◯ | Store DO₁ and N₁ values for future cycle(s) (e.g. DO₁ may be used as DO₀). |

| | |
|---|---|
| [symbol] | Else - keep stirring speed at value from preceding cycle: N₁ = N₀ |

The DO level may further be controlled by adjusting the oxygen supply to the fermentation vessel in addition to, or instead of, adjusting the stirring speed as described above. For example, the oxygen supply (gassing) (G) control signal(s) may be proportional to N₁ as determined above (or to No), the two properties being related by G=N₁×a+b, where a and b are appropriate constants. Constants a and b may depend on the maximum and/or minimum permitted gassing and/or stirring values—e.g. the relationship between gassing (G) and stirring (N₁) may be:

$$G = \frac{{N}_{1} - {N}_{min}}{{N}_{max} - {N}_{min}} \times {G}_{max} ,$$

where Gₘₐₓ is the maximum permitted oxygen flow rate into the fermentation vessel 328. In another example the gassing G is proportional to the stirring control signal N₁ only in a subrange of the stirring range. The gassing G control signal may be applied in addition to, or instead of, the stirring control signal N₁—for the latter, N₁ may be determined as described above but the control signal never applied (the stirring instead being kept at N₀ i.e. at a constant value of N), and gassing G may applied as a control signal after being determined based on N₁ as described above. Examples for adjusting the gassing G depending on the stirring control signal N₁, which in turn depends on the DO, are provided in equations (3a) and (3b) shown below.

$$G = \frac{N \times ( 1 + \frac{( D {O}_{S P} - D {O}_{1} ) - ( D {O}_{1} - D {O}_{0} )}{{DO}_{SP} \times τ} ) - {N}_{min}}{{N}_{max} - {N}_{min}} \times {G}_{max} \qquad ( 3 a )$$

$$G = \frac{N \times ( 1 + \frac{( D {O}_{S P} - D {O}_{1} ) - ( D {O}_{1} - D {O}_{0} )}{{DO}_{SP} \times τ} ) - {N}_{min}}{{N}_{{max}^{-}} 1.3 \times {N}_{min}} \times {G}_{max} \qquad ( 3 b )$$

Compared to equation (3a), equation (3b) has the effect that the maximal gassing Gₘₐₓ is reached before the maximal stirring Nₘₐₓ is reached, instead of maximal gassing Gₘₐₓ being reached concurrently with maximal stirring Nₘₐₓ being reached. Equation (3b) permits the gassing to remain constant at Gₘₐₓ while stirring still increases in the last 15-20% of the stirring range before Nₘₐₓ is reached. It is observed that in some bioprocesses equation (3b) can provide improved DO control.

FIG. 6b provides an example illustrating gassing G against stirring N according to Equation (3b) for three different bioreactor vessels with different Gₘₐₓ, Nₘₐₓ and Nₘₘ. In a vessel 610 with 1 L working volume (Eppendorf® BioFlo® 1 L) gassing increases from 1 vvm (volume per volume per minute) at 700 rpm up to 3 vvm at 1050 rpm and then remains constant at 3 vvm while the agitation increases to Nₘₐₓ at 1200 rpm. In a vessel 612 with 3 L working volume (Eppendorf® BioFlo® 3 L) gassing increases from 1 vvm at 650 rpm up to 2.5 vvm at 1100 rpm and then remains constant at 2.5 vvm while the agitation increases to Nₘₐₓ at 1200 rpm. In a vessel 614 with 0.3 L working volume (Eppendorf® DASbox®) gassing increases from 1 vvm at 950 rpm up to 3.3 vvm at 1850 rpm and then remains constant at 3.3 vvm while the agitation increases to Nₘₐₓ at 2000 rpm.

The gassing (G) control signal may affect the concentration of oxygen in the supplied air and/or the volumetric flow rate of the air supply. Preferably, for ease of operation, the concentration of oxygen is kept constant and it is the volumetric flow rate that is adjusted. This may simplify the oxygen supply as it may require only a single supply (e.g. pump), whereas varying the concentration may require multiple supplies (e.g. one for air and one for a higher oxygen content gas).

Optionally, prior to setting the control signal to G, the controller function may include checking whether G falls within a permitted range (e.g. not below the minimum permitted value (Gₘm) and not above the maximum permitted value (Gₘₐₓ) for the given oxygen supply system and/or fermentation vessel and/or fermentation process). For example, there may be an upper bound to the oxygen supply volumetric flow rate as an excessively high volumetric flow rate may cause cell damage/death due to shear forces and/or excessive foaming (which may require a high concentration of antifoam that could be undesirable for downstream processing).

Preferably, the DO level is controlled via both an agitation control signal (N) and an oxygen supply control signal (G). This may result in a faster/more responsive control than using only N or G, as DO deviations from the setpoint would be counteracted via both agitation and oxygen supply. Further, controlling both G and N may increase the operational range of DO setpoints, and/or reduce shear forces (and thus cell damage/death) in particular at high DO setpoints. Using both agitation and oxygen supply may allow safe operation at both lower DO setpoints (with neither agitation nor oxygen supply below their minimum permitted values) and higher DO setpoints (with neither agitation nor oxygen supply above their maximum permitted values), than may have been possible if only one of agitation or oxygen supply were controlled and the other fixed. Similarly, achieving a high DO setpoint with the control of only one of agitation or oxygen supply may lead to excessive shear forces (e.g. caused by excessive agitation or excessive air flow rate) and thus to cell death, which may not be the case if both agitation and oxygen supply are controlled. Moreover, using both an agitation control signal (N) and an oxygen supply control signal (G) takes advantage of the close correlation in the effectiveness of one control signal on the value of the other. For example, in order for the effects of a large oxygen supply to be maximised, the agitation speed should preferably be at a sufficiently high value to ensure that the large supply of oxygen is mixed/spread throughout the liquid medium 308. On the other hand, if agitation (and thus mixing) is limited, increasing the oxygen supply may have little effect on the DO level, while possibly leading to shear forces that damage cells in the liquid medium 308.

The optional upper and lower bounds for stirring, gassing, and/or other control signal(s) may be adjusted to account for the real working volume of the fermentation vessel. For example, Gₘᵢₙ may be set to a minimum of 1 L of gas flow per minute.

Fermentation Phases

The control module 250 determines 408 the fermentation phase (and/or (detects) transitions between fermentation phases) which may affect the process condition(s) setpoint(s) and/or the control signal(s) for the actuator(s). Preferably, this determination is made at each cycle of method 400.

In more detail, a number of (preferably consecutive) fermentation phases are defined within the control module 250, and the control module 250 evaluates pre-defined conditions to determine the phase of the fermentation process. The control module 250 may further determine control signal(s) for actuator(s) corresponding to the determined phase. In particular, the control module 250 detects a transition from a batch phase to a production phase in the fermentation process (e.g. via method 900 described below). The control module 250 may further implement a transition between a batch phase and a production phase (see e.g. method 1000) and/or between a production phase and the end of the fermentation process (see e.g. method 1100) via control signal(s) for actuator(s) in the fermentation vessel 102 set directly (e.g. the control module 250 may switch on/off a feed supply) and/or indirectly (e.g. the control module 250 may modify setpoints (e.g. for temperature) and leave it to the above-described controller function(s) to achieve those setpoints).

Example process conditions for which setpoints may depend on the fermentation phase include the DO, and temperature. For example, the temperature setpoint may be higher for the batch phase than for the production phase, to balance factors such as optimal cell growth during the batch phase and/or the reducing solubility of oxygen in the liquid medium with increasing temperature. The temperature setpoint may be yet lower during an end of fermentation phase. Similarly, the DO setpoint may be greater during the batch phase than during the production phase and/or during the transition between the phases, in order to promote cell growth during the batch phase, and to protect oxidation-sensitive proteins expressed in the production phase. The DO setpoint may be yet different during the end of fermentation phase.

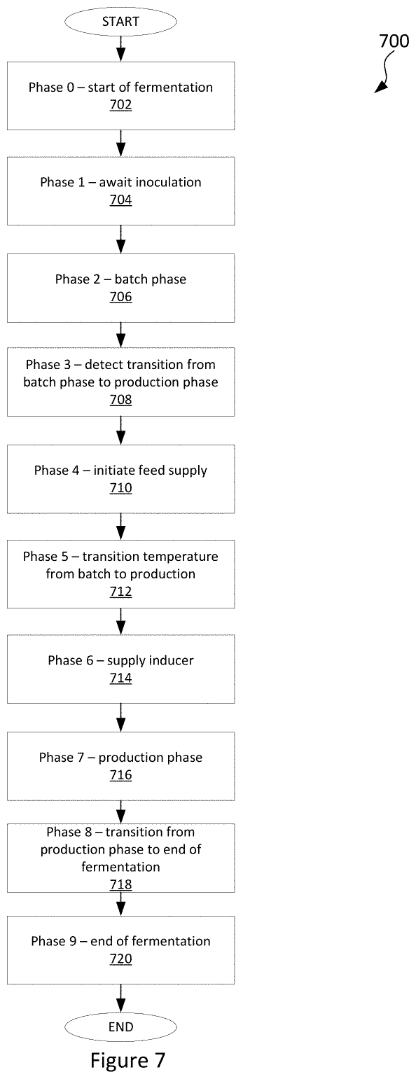

Referring to FIG. 7, an example listing of consecutive fermentation phases 700 defined in the control module 250 is shown. The phases 700 include a batch phase 706 (phase 2), and a production phase 716 (phase 7). Phases 700 may include a number of further phases preceding the batch phase 706, such as: a ‘start of fermentation’ phase 702 (phase 0), and/or a ‘pre/awaiting inoculation’ phase 704 (phase 1). Phases 700 may further comprise phases in-between the batch phase and the transition phase which manage the transition of the fermentation process from a batch phase to a production phase, such as: a ‘detect transition from batch phase to production phase’ phase 706 (phase 3), an ‘initiate feed supply’ phase 710 (phase 4), a ‘transition temperature from batch to production phase’ phase 712 (phase 5), and/or a ‘supply inducer’ phase 714 (phase 6). Finally, phases 700 may further comprise phases following the production phase 716 that control the transition to the end of the fermentation process, such as: a ‘transition from production phase to end of fermentation’ phase 718 (phase 8), and/or an ‘end of fermentation’ phase 720 (phase 9).

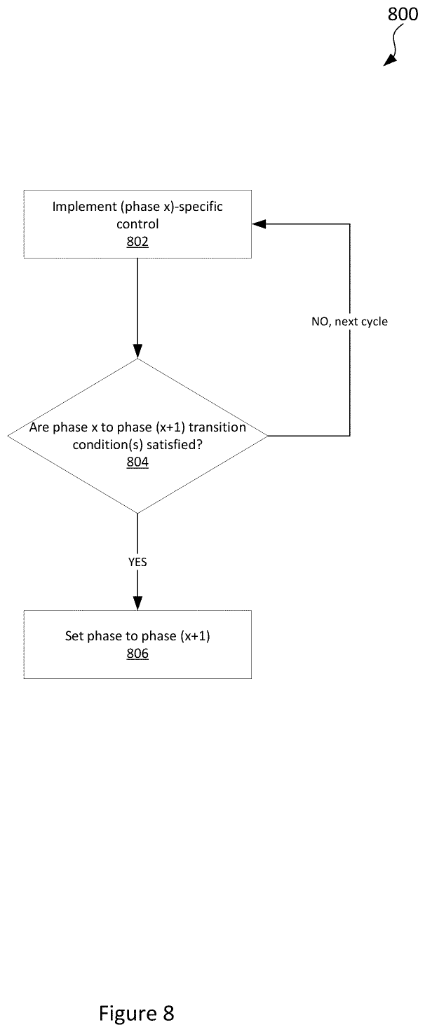

Referring to FIG. 8, an example method 800 of transitioning between one phase (e.g. phase x) and the next phase (e.g. phase (x+1)) among phases 700 is shown. Method 800 indirectly allows determining the fermentation phase, and so can be considered as an implementation of method 408 (noting that method 800 also includes certain aspects of method 406). Each phase (such as phase x) may have associated control signal(s) for actuator(s) (or, in other words, a phase-specific control) that allow controlling the entire fermentation process in the fermentation vessel 102 beyond simply ensuring that process condition(s) are at their setpoint(s). The first step of method 800 is implementing 802 such (phase x)-specific control. For example, phases 710 to 720 (or 4 to 9) may include implementing phase-specific control such as supplying the feed at a given rate or switching off one of the supplies (e.g. acid/base supply). Further details of the phase-specific control implemented in phases 4 to 9 are provided in sections below.

Next, the control module 250 determines 804 whether the condition(s) for transitioning from phase x to phase (x+1) are met. For example, the control module 250 may determine whether the time since the start of phase x (phase x duration) is above a pre-defined threshold.

Example phase transition condition(s) are shown in Table 3 below. The phase condition examples shown in Table 3 are based on thresholds for various process conditions and/or timescales. Thresholds relating to phase duration (the first and eighth thresholds) are typically in the range of 1 to 1000 seconds, and preferably between 10 and 300 seconds (the lower limit being dependent on the sampling time of method 400). Thresholds relating to the amount (e.g. volume) of supplied material (the fifth and seventh thresholds) may be implemented by integrating (e.g. numerically with a sampling rate equal to the control sampling rate) the rate (e.g. volumetric flow rate) at which the material is supplied to obtain the total amount of material supplied up to a cycle and comparing this to the amount of material to be supplied in that phase (the desired amount)—the thresholds. Or, for a simpler case in which the rate at which the material is supplied is constant, the amount of material supplied up to a cycle may be determined as a product of the phase duration to date (e.g. time since phase start) and the rate at which the material is supplied. Phases 5 and 8 are primarily concerned with transitioning the temperature (which may be the temperature in the fermentation vessel 328 and/or the temperature setpoint) from a starting setpoint in one phase (or set of phases) to a target setpoint in another phase (or set of phases). Thus, the sixth and eighth thresholds may depend on (e.g. be proportional to) the target temperature setpoint. For example, if the production phase temperature setpoint (T_{SP,exp}) is lower than the batch phase temperature setpoint, the condition 804 for transition from phase 5 to phase 6 may be: T<T_{SP,exp}+c, where c is a positive constant.

Further details of the transition conditions between phases 2 706 and 3 708, and between phases 3 708 and 4 710 which relate to detecting a transition from a batch phase to a production phase (and/or detecting the end of the batch phase and/or the start of the production phase), and of the thresholds relating to an estimated oxygen consumption are provided in the sections below.

If the condition(s) 804 are met/satisfied, the phase is set 806 to (x+1) for the next cycle. If not, the phase remains set to x and method 800 is repeated at the next cycle.

TABLE 3

| Phase | Condition 804 for transition to next phase |
|---|---|
| 0 | Phase duration above a first threshold |
| 1 | Inoculation completed by external process |
| 2 | Estimated oxygen consumption above a third threshold (and |
| | optionally duration of high (high being determined by a further |
| | threshold) stirring period above a yet further threshold) |
| 3 | Estimated oxygen consumption below a fourth threshold |
| | (wherein the fourth threshold is optionally proportional (or a ratio |
| | of) the third threshold) |
| 4 | Amount (e.g. quantified via volume) of feed supplied to the |
| | fermentation vessel as part of phase 4above a fifth threshold |
| 5 | Temperature below/above a sixth threshold (wherein the sixth |
| | threshold is optionally proportional to the temperature setpoint in |
| | the production phase 7) |
| 6 | Amount (e.g. quantified via volume) of inducer supplied to the |
| | fermentation vessel as part of phase 6 above a seventh threshold |
| 7 | Phase duration above an eighth threshold |
| 8 | Temperature below/above a ninth threshold (wherein the ninth |
| | threshold is optionally proportional to the temperature setpoint in |
| | the end of fermentation phase 9) |

It should be appreciated that the phases 700 have been divided into phases 0-9 as described above for the purpose of clarity. The phases 700 could be ‘rolled into’ fewer phases, or indeed split into further phases. For example, phases 0 to 3 could be rolled into a single batch phase; phases 4 to 6 could be rolled into a ‘transition from batch to production’ phase; and/or phases 8 to 9 could be rolled into a ‘transition from production to fermentation end’ phase.

Detecting Transition from Batch Phase to Production Phase

The control module 250 detects a transition from a batch phase to a production phase by monitoring an estimated oxygen consumption rate within the fermentation vessel 328. Oxygen is a key substrate for cell growth during the batch phase, in particular for an aerobic fermentation process. Thus, as the cell population (and/or density) increases with time during the batch phase, this leads to an increasing demand for oxygen (and/or increasing oxygen consumption). Towards the end of the batch phase the oxygen consumption rate may often exceed the supply (e.g. exceed maximum permitted agitation and/or oxygen supply) and lead to a reduction in the DO level. However, once the initially provided feed (e.g. carbon source) is depleted the cell population growth ceases and the oxygen demand/consumption drops. Thus, a transition from a batch phase to a production phase may be detected by detecting a high oxygen consumption rate followed by a (typically significantly) reduced oxygen consumption rate.

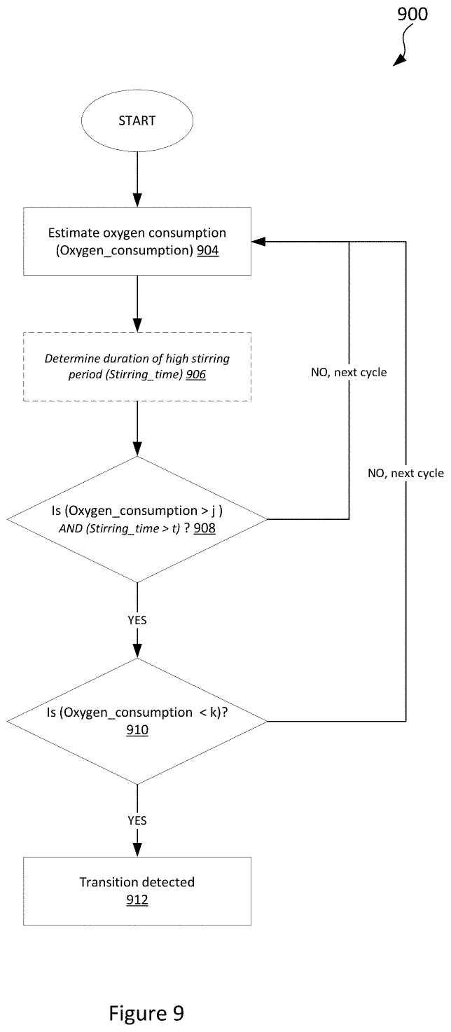

Referring to FIG. 9, an example method 900 of detecting the end of the batch phase is shown. Method 900 may be implemented in practice as a separate/stand-alone function, and/or via transitions between phases as described above—e.g. a transition from a batch phase to a production phase may be detected when the phase is set to phase 4 710 (i.e. when the condition(s) 804 for transition to the next phase are met for phase 2 and subsequently for phase 3). In the latter case, phase 2 may include steps 904 to 908, and phase 3 would include step 904 along with steps 910 to 912.

The control module 250 first determines 904 an estimate of the oxygen consumption (rate) (oxygen_consumption) in the fermentation vessel 328 and/or fermentation vessel 102. In the present example, the oxygen consumption rate is estimated as a ratio

$$\frac{O}{D O}$$

of an oxygenation parameter O that reflects oxygenation actuator control signal(s) O (e.g. agitation speed and/or oxygen supply) which when increased act to increase the DO level, to the DO level. For example, the oxygen consumption rate may be estimated as:

$$oxygen\_consumption = \frac{N}{D O} , \qquad ( 4 )$$

$$oxygen\_consumption = \frac{G}{D O} , \qquad ( 5 )$$

$$oxygen\_consumption = \frac{N + G}{D O} , or \qquad ( 6 )$$

$$oxygen\_consumption = \frac{N \times G}{D O} . \qquad ( 7 )$$

Where, in the equations above, N is the agitation speed, and G is a process condition related to the oxygen supply (e.g. volumetric flow rate of supplied gas, or oxygen concentration in the supplied gas, or a combination of these values). N and/or G and/or DO in these example equations may be scaled so that they are non-dimensional—e.g. N and G may be scaled by their maximum or minimum permitted values, and DO may be scaled by its setpoint.

Although estimating the oxygen consumption rate using such a ratio does not provide a physical value for the oxygen consumption rate, it provides a relative measure that can be used to compare the oxygen consumption rates at various stages in the fermentation process, and in particular during the batch phase of the fermentation process. For example, if at point (1), e.g. near the start of the batch phase, DO is kept at its setpoint with a relatively low stirring speed N, and at point (2), e.g. near the end of the batch phase, the DO level has fallen below that same setpoint despite a high stirring speed (e.g. maximum permitted stirring speed), this gives an indication that the oxygen consumption rate is greater at point (2) than point (1), since at point (2) actuator action (increased stirring speed N) that would be expected to increase the DO level is not doing so which suggests that oxygen is consumed at a higher rate at point (2).

Instead of estimating the oxygen consumption rate using a ratio as described in detail above, other mathematical relationships between the oxygenation parameter O (e.g. agitation speed and/or oxygen supply) and the DO level can be used to estimate the oxygen consumption rate analogous to the described ratio, with suitable thresholds determined as appropriate. For example, a difference (O−DO) can suitably provide an estimate of the oxygen consumption rate; scaling of one of the parameters can assist in providing a convenient aggregate parameter (e.g. stirring is typically in hundreds and thousands rpm, while DO is in tens of %). A logarithmic relationship (e.g. log_{DO}(O)) or exponential relationship (e.g.

$${DO}^{\frac{1}{O}} = \sqrt[O]{DO} )$$

can similarly provide an estimate of the oxygen consumption rate.

As described above, the DO control where DO is controlled depending on the foregoing DO value (e.g. equations (2a) and (2b) above) can have a dampening or anticipatory effect (in particular when approaching the setpoint; the more rapidly the DO value has recently changed, the greater the controlling or dampening effect). Detecting a spike in the DO controlled in this way by a conventional approach (delta DO above a threshold) can be problematic, as the control is designed to suppress and prevent spiking; due to the underlying system spiking will occur, but detection of the spike is not an optimal marker for transitioning of the system with such a control regime. Estimating the oxygen consumption rate as described above is found to be more effective in detecting transitioning of the system when the DO control includes dampening/anticipatory functionality.

Once the oxygen consumption rate is estimated 904, the control module 250 compares 908 this estimated oxygen consumption rate against a (third) threshold, j, which corresponds to an (relatively high) oxygen consumption rate at (or near) the end of the batch phase.

If the oxygen consumption rate is below threshold (j), this implies that the batch phase is still in an early stage and not yet approaching its peak, and method 900 terminates—starting once again from step 902 at the next cycle.

If the oxygen consumption is above threshold (j), this implies that the fermentation process is at (or approaching) the end of the batch phase, and the control module 250 begins monitoring (preferably starting at the next cycle) for a drop in the estimated oxygen consumption rate which would imply that the carbon source is depleted and it is appropriate to start the production phase. The control module 250 does this by comparing 910 the estimated oxygen consumption rate against a (fourth) threshold, k, which corresponds to an (relatively low) oxygen consumption rate at (or near) the start of the production phase.

If the oxygen consumption is above threshold (k), this implies that the microorganism population is still growing and the carbon source is not exhausted yet, and so transitioning from a batch phase to a production phase would be premature, and method 900 terminates—starting once again from step 902 at the next cycle.

If the oxygen consumption is below threshold (k), this implies that a transition in population growth behaviour is occurring due to the carbon source being at or near exhaustion, and so it is appropriate to start the transition from a batch phase to a production phase.

Threshold j is preferably greater than threshold k. Further, threshold j may depend on threshold k and vice versa. For example, threshold k may be proportional (or be scaled with respect) to threshold j—e.g. j=2k. This may simplify the process of determining thresholds j, k as only one of the thresholds would need to be determined (e.g. empirically/experimentally).

Optionally, at step 908, the control module further checks whether a second estimated oxygen consumption rate is relatively high, e.g. by comparing a second of the numerators of equations (4) to (7) Including N and/or G, or a combination of thereof) against a threshold (e.g. proportional to the maximum permitted agitation speed), optionally for a given duration of time (which may further improve the reliability as it may eliminate short term “outliers”).

Thresholds j, k, and/or t described above, as well as the thresholds in Table 3, may be determined empirically, by testing the control module 250 with multiple threshold values and determining the optimal one(s)—e.g. the threshold value(s) j, k, and/or t that are the most reliable in detecting a transition from a batch phase to a production phase. The thresholds may depend on the fermentation process (in particular on the medium and/or output product (e.g. target protein) composition), and/or on the fermentation vessel 102 (e.g. maximum/minimum agitation speed and/or oxygen supply (gassing), or fermentation vessel (working) volume) and/or control unit 104 properties.

Transitioning from Batch Phase to Production Phase

In addition to detecting a transition from a batch phase to a production phase (e.g. via method 900 described above), the control module 250 may be configured to automate the transition/switching between phases itself by determining and providing the appropriate actuator control signal(s) to exit a batch phase and/or initialise a production phase.

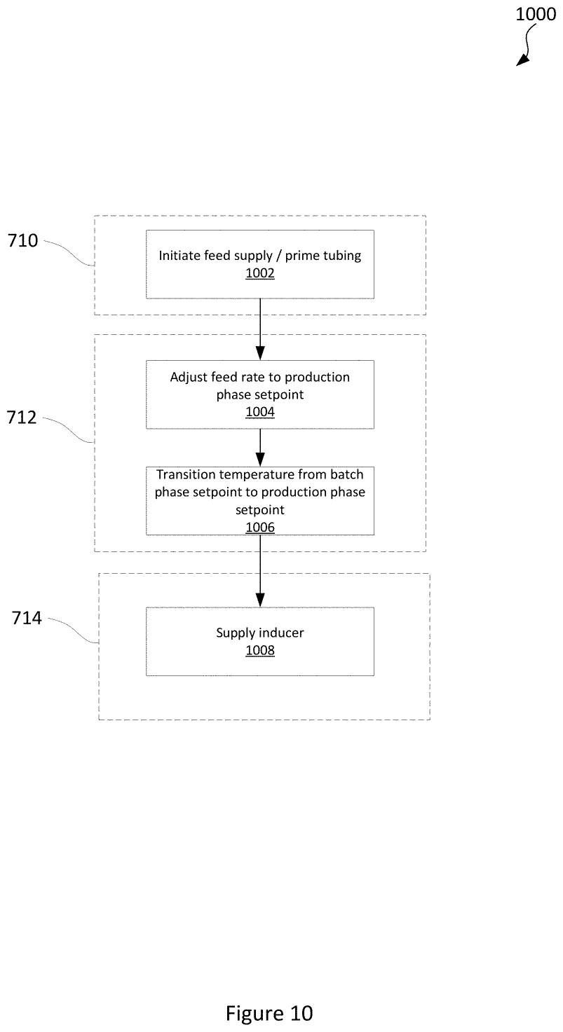

Referring to FIG. 10, an example method 1000 of transitioning between a batch phase and a production phase is shown. Method 1000 preferably follows method 900 (e.g. starts in the next cycle after a transition 912 in population growth behaviour is detected). The steps in FIG. 10 may be performed as part of phases 4 710, 5 712, and 6 714 as shown in FIG. 10. Thus, the sets of steps in each phase may be repeated multiple times (and/or run in parallel) before the steps in the next phase are performed.

Following detection of a transition 912 in population growth behaviour due to carbon source exhaustion, first, feed (e.g. carbon source) is supplied 1002 to the fermentation vessel 328 in order to avoid starvation of the cells in the medium and to provide an optimum environment for the production phase (e.g. optimum environment for target protein expression). Step 1002 also results in priming of the tubing in the feed supply (removing air that may reside in the tubing), so that the feed supply may be uninterrupted in later during the production phase. The feed supplied at step 1002 is preferably a feed shot—a relatively large amount (as measured by volume or weight or number of moles) supplied over a relatively short period of time (e.g. at a maximum permitted feed supply rate). Preferably, the feed is supplied 1002 until a pre-defined amount has been supplied to the fermentation vessel 328.

Once the feed shot is supplied 1002, the feed rate is adjusted 1004 to its production phase setpoint (e.g. as defined by the feed supply volumetric flow rate (L/h)). The feed rate is preferably maintained at this level to the end of the production phase (e.g. throughout phases 712, 714 and 716).

Optionally, the feed rate is further adjusted (i.e. corrected) based on a calibration of the feed pump for the given vessel. For example, the feed rate may be corrected using a linear regression method. The relationship (which is assumed to be represented by a straight line—i.e. coefficients a and b in y=ax+b) between the corrected feed rate and the feed rate production phase setpoint is determined (e.g. experimentally and/or using computational (e.g. machine-learning) modelling), which is subsequently used to determine the corrected feed rate based on a desired feed rate production phase setpoint.

Next, the temperature is changed 1006 from a batch phase setpoint to a production phase setpoint (e.g. by changing the temperature setpoint which is then achieved via the heating/cooling system controller function). Preferably, this change is performed gradually, relatively slowly and in a relatively stepless manner (with no sudden jumps/changes in temperature) so as to provide a slower/smoother change in temperature. For example, the temperature setpoint may be changed at a linear rate dependent on the batch and production phase setpoints and a pre-defined duration of the temperature transition period. This may avoid (or reduce the frequency/possibility of) hot/cold spots (areas of significantly increased/decreased temperature) being generated near the heating/cooling interface of the heating/cooling system in the vessel (e.g. in the medium near the surface of the fermentation vessel 328 if a heating/cooling jacket is used as the heating/cooling system). Such hot/cold spots may be detrimental to target protein expression and/or lead to cell damage/death. If the temperature transition were too fast, the heating/cooling system would need to set its heating/cooling elements to a very high/low temperature and thus generate hot/cold spots in the medium adjacent (or nearby) to the heating/cooling elements.

Finally, an inducer is supplied 1008 in order to improve and/or facilitate chemical and/or biological processes (e.g. transcription and gene expression) in the production phase. Preferably, similar to the feed supplied at step 1002, the inducer is supplied 1008 as a “shot”—a relatively large amount (as measured by volume or weight or number of moles) supplied over a relatively short period of time (e.g. at a maximum permitted feed supply rate). Preferably, the inducer is supplied 1008 until a pre-defined amount (e.g. volume) has been supplied to the fermentation vessel 328, at which point the inducer supply is stopped/switched off.

Optionally, the material supply rate(s) (e.g. oxygen supply rate, feed supply rate, and/or inducer supply rate) are adjusted to account for the real working volume of the fermentation vessel (the working volume being a fraction of the total volume taken up by the medium (and materials within the medium such as gas bubbles), thus discounting the remaining headspace volume). For example, the control module 250 may have a pre-defined target feed supply rate (e.g. measured in grams of feed per hour, per 1 L of working volume), feed_target, and determine the feed supply rate, feed_supply_rate corresponding to this target based on the working volume, V, and the feed concentration, C, in the feed supply—e.g. via feed_supply_rate=(feed_target*V)/C. Preferably, this adjustment is performed at each cycle that the material (e.g. feed) is supplied. This adjustment may be performed in one or more of any of phases 0 702 to 9 720.

Transitioning from Production Phase to End of Fermentation

The control module 250 may further be configured to automate a transition from a production phase to an end of fermentation phase. The end of the production phase may for example be detected via comparing the production phase time to a pre-defined threshold (e.g. eighth threshold in Table 3).

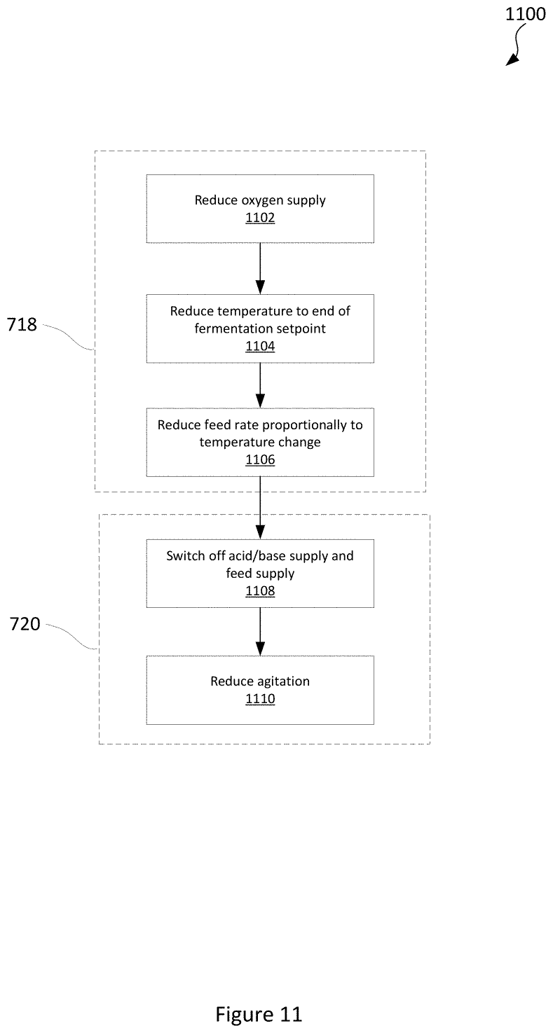

Referring to FIG. 11, an example method 1100 of transitioning between a production phase and an end of fermentation phase is shown. Method 1100 preferably follows the production phase 7 716 (e.g. starts in the next cycle after the condition(s) 804 for phase transition to phase 8 718 are met). The steps in FIG. 11 may be performed as part of phases 8 718, and 9 720 as shown in FIG. 7. Thus, the sets of steps in each phase may be repeated multiple times (and/or run in parallel) before the steps in the next phase are performed.

Method 1100 has been designed in order to perform a smooth transition from production phase process conditions (e.g. aimed at promoting target protein expression) to those at the end of the fermentation process (e.g. aimed at conditioning of the expressed target protein), while simultaneously reducing/avoiding cell death (e.g. due to excessive shear forces or starvation).

As the first step, the oxygen supply is reduced 1102. This reduction may correspond to a reduction in the oxygen concentration in the oxygen supply and/or the flow rate of the oxygen supply. Preferably, the reduction is solely or primarily in the flow rate of the oxygen supply in order to limit the shear forces in the medium. Following the production phase, the oxygen consumption in the fermentation vessel 328 is typically significantly reduced so, in order to conserve resources and avoid oxygen supply-caused cell death, the oxygen supply may be reduced. However, the oxygen consumption does not typically drop to zero, so the oxygen supply is preferably reduced to a non-zero, relatively low, value that is sufficiently high to avoid/reduce cell death due to starvation—e.g. around 1 vvm (1 L of air passing through 1 L of medium per minute).

Subsequently, the temperature is transitioned 1104 from a production phase setpoint to a, typically lower, end of fermentation setpoint. For example, the temperature may be transitioned 1104 by setting the temperature setpoint to the end of fermentation setpoint and allowing the temperature controller function to achieve this temperature in the fermentation vessel 328. In order to avoid/reduce final cell starvation, the feed is preferably reduced 1106 proportionally to the temperature transition 1104. For example, the feed rate may be reduced from its production phase value to a lower value (preferably zero) at the same rate as the temperature in the fermentation vessel 328 is transitioned from its production phase setpoint to its end of fermentation setpoint. Steps 1104 and 1106 are preferably run in parallel and/or repeated one after the other over multiple cycles.

Next, the material supply systems (e.g. the feed and acid/base supplies) are switched off 1108. Optionally, ahead of step 1108, the acid/base supply may be gradually reduced similarly to the feed supply reduction described above.

Finally, the agitation (e.g. agitation/stirring speed) is reduced 1110 to a relatively low level in order to reduce shear forces caused by the agitation, while ensuring mixing of the likewise relatively low amount of oxygen supplied to the fermentation vessel 328 in order to reduce/avoid starvation. Agitation is preferably reduced 1110 last (as shown in FIG. 11) in order to reduce/eliminate gradients of concentration in the liquid medium as the process conditions are modified in steps 1102 to 1108—e.g. to reduce DO, temperature, feed, and/or acid/base concentration gradients.

Example Bioprocess: Botulinum Neurotoxins

While the described control can be applied to a wide variety of bioprocesses, clostridial neurotoxins are now described in more detail as an example of a product that can be produced with a bioprocess controlled using the control method described above.

Bacteria in the genus Clostridia produce highly potent and specific protein toxins, which can poison neurons and other cells to which they are delivered. Examples of such clostridial toxins include the neurotoxins produced by C. tetani (TeNT) and by C. botulinum (BoNT) serotypes A-G, as well as those produced by C. baratii and C. butyricum.

Clostridial neurotoxins (for example, in nature) cause muscle paralysis by inhibiting cholinergic transmission in the peripheral nervous system, in particular at the neuromuscular junction, and can thus be lethal. In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site, which is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (HN domain) and a C-terminal targeting component (HC domain). The cleavage site is located between the L-chain and the HN domain.

The mode of action of clostridial neurotoxins relies on five distinct steps: (1) binding of the HC domain to the cell membrane of its target neuron, followed by (2) internalisation of the bound toxin into the cell via an endosome, (3) translocation of the L-chain by the HN domain across the endosomal membrane and into the cytosol, (4) proteolytic cleavage of intracellular transport proteins known as SNARE proteins by the L-chain which provides a non-cytotoxic protease function, and (5) inhibition of cellular secretion from the target cell.

Non-cytotoxic proteases act by proteolytically cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

Thanks to their unique properties, Clostridial neurotoxins, such as botulinum toxin, have been successfully employed in a wide range of therapeutic applications, in particular for motor and autonomic disorders, to restore for example the activity of hyperactive nerve endings to normal levels. At least seven antigenically distinct BoNTs serotypes have been described so far, namely BoNT/A, BoNT/B, BoNT/C, BoNT/D, BoNT/E, BoNT/F, BoNT/G (Rossetto, O. et al., “Botulinum neurotoxins: genetic, structural and mechanistic insights.” Nature Reviews Microbiology 12.8 (2014): 535-549).

Despite this diversity, BoNT/A remains the serotype of choice in therapy, with three commonly available commercial preparations (Botox®, Dysport® and Xeomin®), while only one BoNT/B product is available on the market (Neurobloc®/Myobloc®). To this day, these BoNT/A and BoNT/B products, which are toxins purified from clostridial strains, are the only two BoNT serotypes that are currently approved by regulatory agencies for use in humans for applications ranging, among others, from spasticity, bladder dysfunction, or hyperhidrosis (for BoNT/A) (see for example: https://www.medicines.org.uk/emc/medicine/112, https://www.medicines.org.uk/emc/medicine/870, https://www.medicines.org.uk/emc/medicine/2162, herein incorporated by reference in their entirety) to cervical dystonia (for BoNT/B) (see for example, https://www.medicines.org.uk/emc/medicine/20568, herein incorporated by reference in its entirety).

In contrast to a cytotoxic protease (e.g. ricin, diphtheria toxin, pseudomonas exotoxin), which acts by killing its natural target cell, clostridial neurotoxins are non-cytotoxic proteases acting by transiently incapacitating the cellular function of its natural target cell. Importantly, a non-cytotoxic protease does not kill the natural target cell upon which it acts. In addition to clostridial neurotoxins (e.g. botulinum neurotoxin, marketed under names such as Dysport™, Neurobloc™, and Botox™), some of the best-known examples of non-cytotoxic proteases include IgA proteases (see, for example, WO99/032272), and antarease proteases (see, for example, WO2011/022357).

The term “clostridial neurotoxin” as used herein means any polypeptide that enters a neuron and inhibits neurotransmitter release. This process encompasses the binding of the neurotoxin to a low or high affinity receptor, the internalisation of the neurotoxin, the translocation of the endopeptidase portion of the neurotoxin into the cytoplasm and the enzymatic modification of the neurotoxin substrate. More specifically, the term “neurotoxin” encompasses any polypeptide produced by Clostridium bacteria (clostridial neurotoxins) that enters a neuron and inhibits neurotransmitter release, and such polypeptides produced by recombinant technologies or chemical techniques. Preferably, the clostridial neurotoxin is a botulinum neurotoxin (BoNT).

At least seven antigenically distinct BoNTs serotypes have been described so far, namely BoNT/A, BoNT/B, BoNT/C, BoNT/D, BoNT/E, BoNT/F, BoNT/G (Rossetto, O. et al., “Botulinum neurotoxins: genetic, structural and mechanistic insights.” Nature Reviews Microbiology 12.8 (2014): 535-549).

BoNT serotypes A to G can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

An example of a BoNT/A neurotoxin amino acid sequence is provided as SEQ ID NO: 1 (UniProt accession number PODP11) or as SEQ ID NO: 11 (UniProt accession number PODP10). An example of a BoNT/B neurotoxin amino acid sequence is provided as SEQ ID NO: 2 (UniProt accession number B INP5). An example of a BoNT/C neurotoxin amino acid sequence is provided as SEQ ID NO: 3 (UniProt accession number P18640). An example of a BoNT/D neurotoxin amino acid sequence is provided as SEQ ID NO: 4 (UniProt accession number P19321). An example of a BoNT/E neurotoxin amino acid sequence is provided as SEQ ID NO: 5 (NCBI Reference Sequence, accession number WP_003372387). An example of a BoNT/F neurotoxin amino acid sequence is provided as SEQ ID NO: 6 (UniProt accession number Q57236) or as SEQ ID NO: 9 (UniProt/UniParc accession number UPI0001DE3DAC). An example of a BoNT/G neurotoxin amino acid sequence is provided as SEQ ID NO: 7 (accession number WP_039635782). An example of a BoNT/D-C neurotoxin amino acid sequence is provided as SEQ ID NO: 8 (UniProt accession number C6KZT4). An example of a BoNT/X neurotoxin amino acid sequence is provided as SEQ ID NO: 10 (UniProt accession number PODPK1).

The clostridial neurotoxin of the present invention can be produced using recombinant technologies. Thus, in one embodiment, the clostridial neurotoxin of the invention is a recombinant clostridial neurotoxin.

In one embodiment, the clostridial neurotoxin of the invention is a Botulinum neurotoxin comprising one or more nucleic acid or amino acids mutations.

In one embodiment, the clostridial neurotoxin of the invention is a chimeric Botulinum neurotoxin.

In an exemplary bioprocess a clostridial neurotoxin is produced by recombinant E. coli. Briefly, DNA constructs encoding the desired clostridial neurotoxin are synthesised, cloned into a suitable vector and then transformed into a suitable strain of E. coli cells for over-expression. The E. coli is cultivated in a fed batch fermentation bioprocess as described above for over-expression of the desired clostridial neurotoxin. The clostridial neurotoxin can be purified from the E. coli lysates using conventional techniques. Such a bioprocess can be used for production of a wide variety of clostridial neurotoxins.

In one embodiment, the clostridial neurotoxin of the invention may be one or more selected from SEQ ID NOs: 1 to 11.

The clostridial neurotoxin of the invention may be a BoNT/A neurotoxin. In one embodiment the clostridial neurotoxin of the invention comprises a polypeptide sequence having at least 70% sequence identity to any one of SEQ ID NOs: 1 or 11. In one embodiment a clostridial neurotoxin of the present invention may comprise a polypeptide sequence having at least 80%, at least 90%, at least 95%, at least 96%, at least 97%, at least 98%, or at least 99% sequence identity to any one of SEQ ID NOs: 1 or 11. Preferably, a clostridial neurotoxin of the present invention may comprise (or more preferably, consist of) a polypeptide sequence shown in any one of SEQ ID NOs: 1 or 11.

The clostridial neurotoxin of the invention may be a BoNT/B neurotoxin. In one embodiment the clostridial neurotoxin of the invention comprises a polypeptide sequence having at least 70% sequence identity to SEQ ID NO: 2. In one embodiment a clostridial neurotoxin of the present invention may comprise a polypeptide sequence having at least 80%, at least 90%, at least 95%, at least 96%, at least 97%, at least 98%, or at least 99% sequence identity to SEQ ID NO: 2. Preferably, a clostridial neurotoxin of the present invention may comprise (or more preferably, consist of) a polypeptide sequence shown in SEQ ID NO: 2.

The clostridial neurotoxin of the invention may be a BoNT/C neurotoxin. In one embodiment the clostridial neurotoxin of the invention comprises a polypeptide sequence having at least 70% sequence identity to SEQ ID NO: 3. In one embodiment a clostridial neurotoxin of the present invention may comprise a polypeptide sequence having at least 80%, at least 90%, at least 95%, at least 96%, at least 97%, at least 98%, or at least 99% sequence identity to SEQ ID NO: 3. Preferably, a clostridial neurotoxin of the present invention may comprise (or more preferably, consist of) a polypeptide sequence shown in SEQ ID NO: 3.

The clostridial neurotoxin of the invention may be a BoNT/D neurotoxin. In one embodiment the clostridial neurotoxin of the invention comprises a polypeptide sequence having at least 70% sequence identity to SEQ ID NOs: 4. In one embodiment a clostridial neurotoxin of the present invention may comprise a polypeptide sequence having at least 80%, at least 90%, at least 95%, at least 96%, at least 97%, at least 98%, or at least 99% sequence identity to SEQ ID NO: 4. Preferably, a clostridial neurotoxin of the present invention may comprise (or more preferably, consist of) a polypeptide sequence shown in SEQ ID NO: 4.

The clostridial neurotoxin of the invention may be a BoNT/E neurotoxin. In one embodiment the clostridial neurotoxin of the invention comprises a polypeptide sequence having at least 70% sequence identity to SEQ ID NO: 5. In one embodiment a clostridial neurotoxin of the present invention may comprise a polypeptide sequence having at least 80%, at least 90%, at least 95%, at least 96%, at least 97%, at least 98%, or at least 99% sequence identity to SEQ ID NO: 5. Preferably, a clostridial neurotoxin of the present invention may comprise (or more preferably, consist of) a polypeptide sequence shown in SEQ ID NO: 5.

The clostridial neurotoxin of the invention may be a BoNT/F neurotoxin. In one embodiment the clostridial neurotoxin of the invention comprises a polypeptide sequence having at least 70% sequence identity to any one of SEQ ID NOs: 6 or 9. In one embodiment a clostridial neurotoxin of the present invention may comprise a polypeptide sequence having at least 80%, at least 90%, at least 95%, at least 96%, at least 97%, at least 98%, or at least 99% sequence identity to any one of SEQ ID NOs: 6 or 9. Preferably, a clostridial neurotoxin of the present invention may comprise (or more preferably, consist of) a polypeptide sequence shown in any one of SEQ ID NOs: 6 or 9.

The clostridial neurotoxin of the invention may be a BoNT/G neurotoxin. In one embodiment the clostridial neurotoxin of the invention comprises a polypeptide sequence having at least 70% sequence identity to SEQ ID NO: 7. In one embodiment a clostridial neurotoxin of the present invention may comprise a polypeptide sequence having at least 80%, at least 90%, at least 95%, at least 96%, at least 97%, at least 98%, or at least 99% sequence identity to SEQ ID NO: 7. Preferably, a clostridial neurotoxin of the present invention may comprise (or more preferably, consist of) a polypeptide sequence shown in SEQ ID NO: 7.

The clostridial neurotoxin of the invention may be a BoNT/D-C neurotoxin. In one embodiment the clostridial neurotoxin of the invention comprises a polypeptide sequence having at least 70% sequence identity to SEQ ID NO: 8. In one embodiment a clostridial neurotoxin of the present invention may comprise a polypeptide sequence having at least 80%, at least 90%, at least 95%, at least 96%, at least 97%, at least 98%, or at least 99% sequence identity to SEQ ID NO: 8. Preferably, a clostridial neurotoxin of the present invention may comprise (or more preferably, consist of) a polypeptide sequence shown in SEQ ID NO: 8.

The clostridial neurotoxin of the invention may be a BoNT/X neurotoxin. In one embodiment the clostridial neurotoxin of the invention comprises a polypeptide sequence having at least 70% sequence identity to SEQ ID NO: 10. In one embodiment a clostridial neurotoxin of the present invention may comprise a polypeptide sequence having at least 80%, at least 90%, at least 95%, at least 96%, at least 97%, at least 98%, or at least 99% sequence identity to SEQ ID NO: 10. Preferably, a clostridial neurotoxin of the present invention may comprise (or more preferably, consist of) a polypeptide sequence shown in SEQ ID NO: 10.

Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position— Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van WaIIe et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}\begin{matrix}[ length of the longer sequence plus the \\ number of gaps introduced into the longer\end{matrix} \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

Conservative Amino Acid Substitutions

Basic: arginine
lysine
histidine

Acidic: glutamic acid
aspartic acid

Polar: glutamine
asparagine

Hydrophobic: leucine
isoleucine
valine

Aromatic: phenylalanine
tryptophan
tyrosine

Small: glycine
alanine
serine
threonine
methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

### Examples

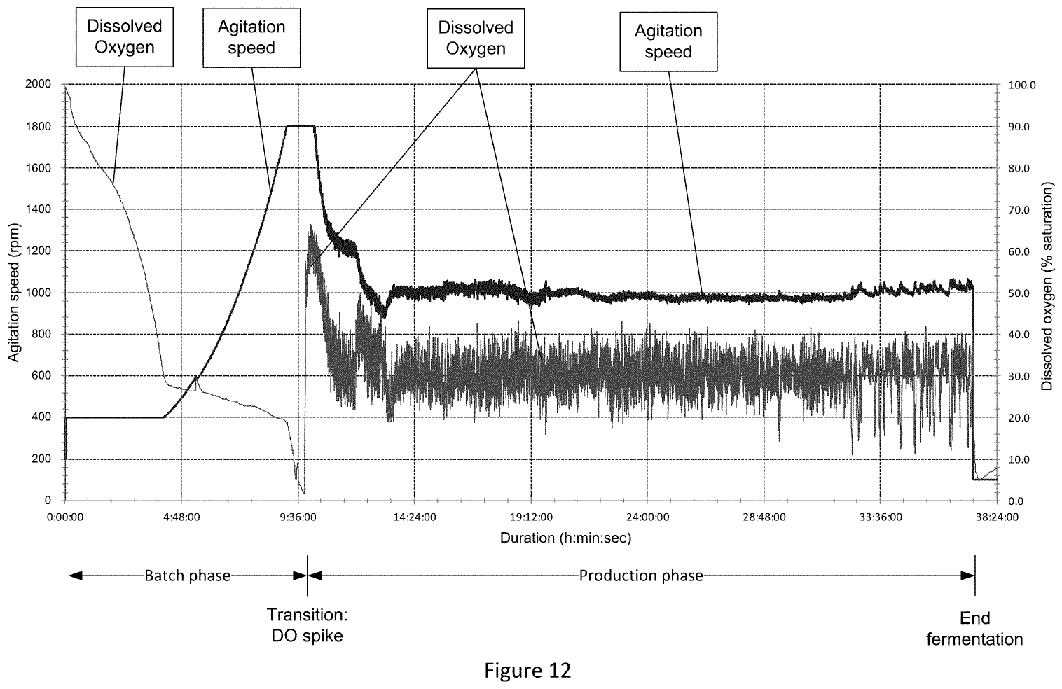

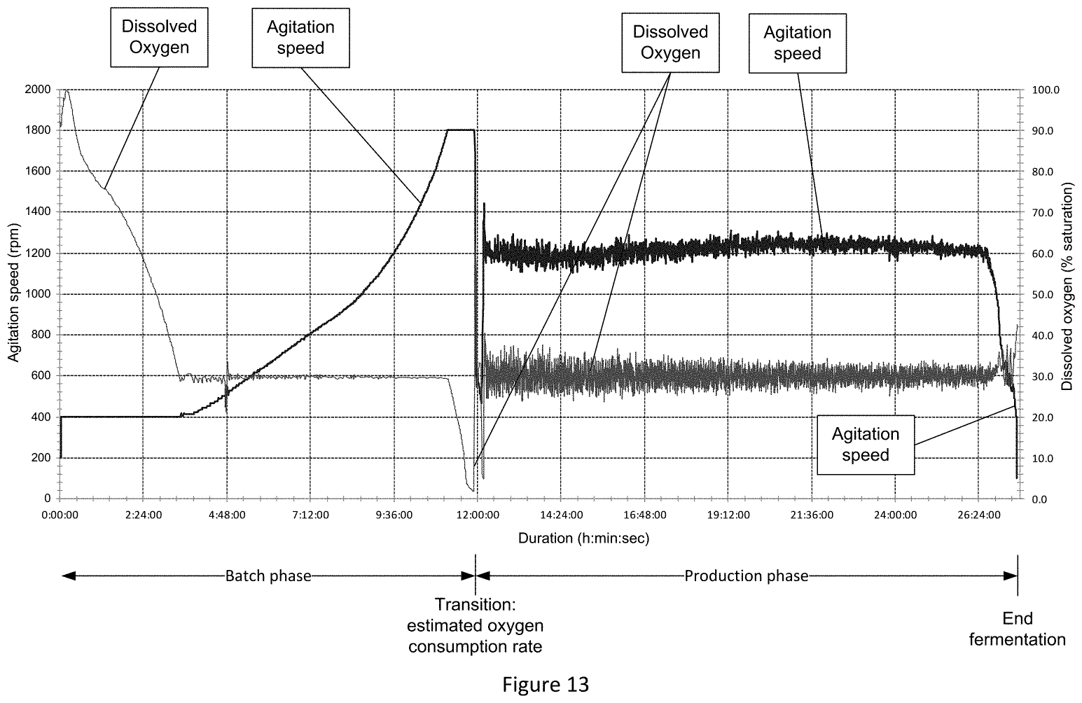

FIG. 12 shows agitation rate and measured dissolved oxygen over the course of a fermentation process controlled by conventional cascade driven DO control. FIG. 13 shows agitation rate and measured dissolved oxygen over the course of the same bioprocess controlled by the control method illustrated in FIGS. 5-11 and as described above. In particular, in the example illustrated in FIG. 13 the agitation system stirring rate/speed (N) in dependence on DO is controlled using equation (2a) above, and the oxygen consumption rate is estimated using the ratio between the agitation rate (N) and the measured DO using equation (4) above.

In the example illustrated in FIG. 13 relatively stable DO control is provided, with smaller fluctuations and deviations from the setpoint. In this example the DO setpoint is at 30% in the batch phase and the production phase, and the measured DO value is within a range of approximately 25% to 35%, so DO conditions are maintained at 30%+/−5%. By contrast, in the example illustrated in FIG. 12 with a conventional controller, and the same DO setpoint at 30%, the measured DO value is within a larger range of approximately 15% to 45%, with DO conditions being maintained at 30%+/−15%. In the example illustrated in FIG. 12 with a conventional controller it is also observed that in the lead up to the phase transition, while the agitation rate is increasing, the measured DO is not well maintained at the setpoint level, but gradually falls below the DO setpoint. By contrast, in the example illustrated in FIG. 13 with a controller as described above, the measured DO is maintained more level near the setpoint level as the batch phase progresses, until the maximum agitation is reached, and only once no further agitation increase is available does the measured DO start to consistently fall below the setpoint level.

Further, with the control method described above, in the transition from batch phase to production phase smoother control of the process is provided. An extract of the plotted values in the transition regions are provided in Tables 4 and 5 below.

TABLE 4
extract of plotted values in transition region from FIG. 12,
with a conventional control method where DO spike detection
starts after e.g. 6:00:00 and is deemed to occur when the DO
value reaches 60%. The agitation speed remains at around 1800
until around 10:07:23, at which time the DO value is at around
58%, having reached a peak value of 66.3% and falling again

| Time | Dissolved oxygen (%) | Agitation speed (rpm) |
|---|---|---|
| 9:46:22 | 2.7 | 1800 |
| 9:50:53 | 2.0 | 1800 |
| 9:52:53 | 2.0 | 1800 |
| 9:53:23 | 12.2 | 1800 |
| 9:53:53 | 30.2 | 1800 |
| 9:54:23 | 41.8 | 1800 |
| 9:54:53 | 53.9 | 1800 |

TABLE 5
extract of plotted values in transition region from FIG.
13, with the control method of the present disclosure.

| Time | Dissolved oxygen (%) | Agitation speed (rpm) |
|---|---|---|
| 11:46:24 | 2.7 | 1800 |
| 11:50:24 | 2.1 | 1800 |
| 11:53:54 | 1.8 | 1800 |
| 11:54:24 | 6.6 | 1800 |
| 11:54:54 | 29.3 | 1800 |
| 11:55:24 | 47.0 | 1687 |
| 11:55:54 | 57.0 | 1411 |

With the control method of the present disclosure the onset of carbon source depletion and with that an optimal time for transition between phases is detected relatively early, within minutes of the dissolved oxygen starting to increase.

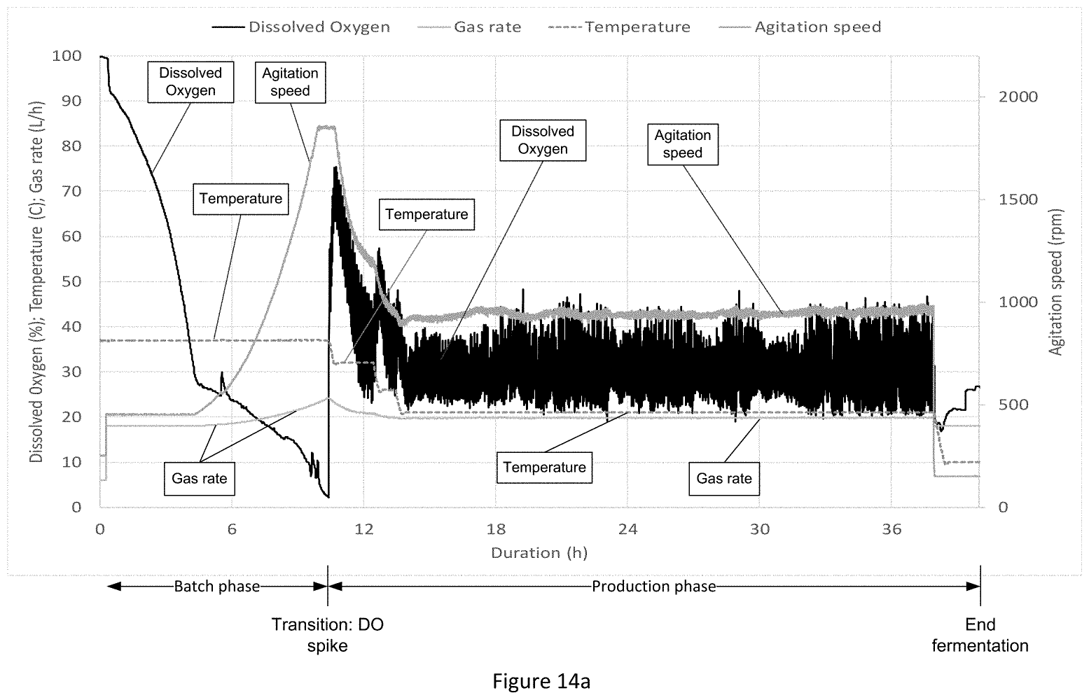

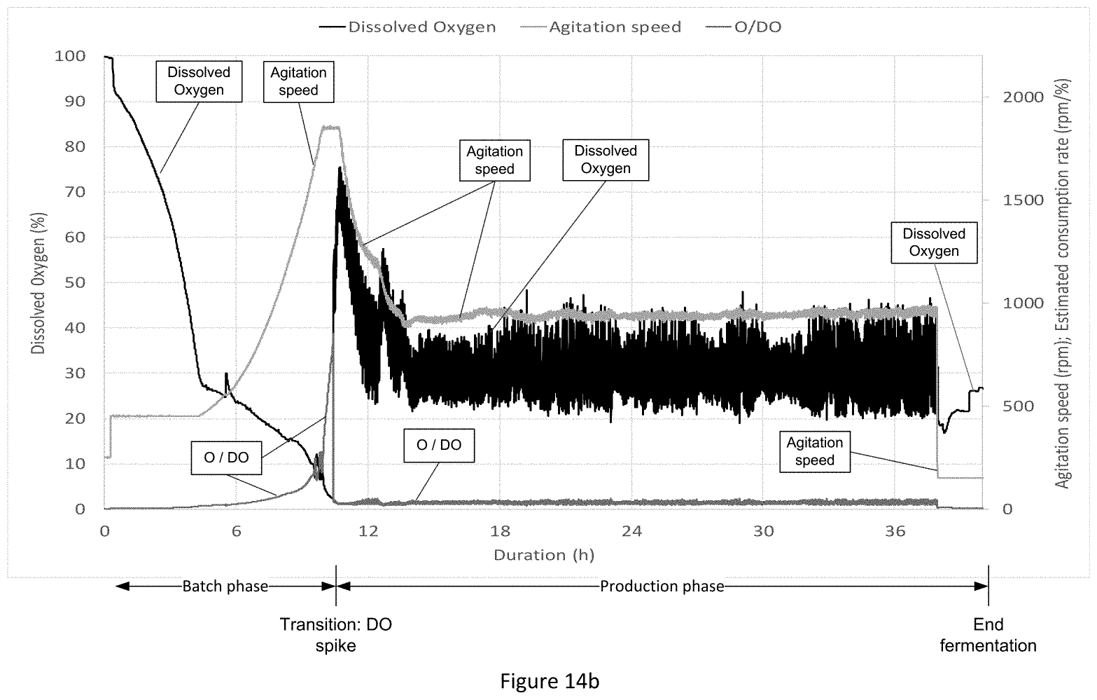

FIGS. 14a to 22 show data from a number of examples with different control methods. In all the illustrated examples the oxygen supply concentration is constant throughout and the gas supply rate and agitation speed are varied.

FIGS. 14a and 14b show agitation rate, measured dissolved oxygen (DO), gas rate (gassing, G), and measured temperature over the course of a fermentation process controlled by conventional cascade driven DO control. For demonstrative purposes, FIG. 14b further shows estimated (oxygen) consumption (rate) (“O/DO”; as determined using the ratio between the agitation rate (N) and the measured DO using equation (4) above), which was not be part of the conventional cascade driven DO control (where a DO spike rather than estimated oxygen consumption was used for phase transitioning).

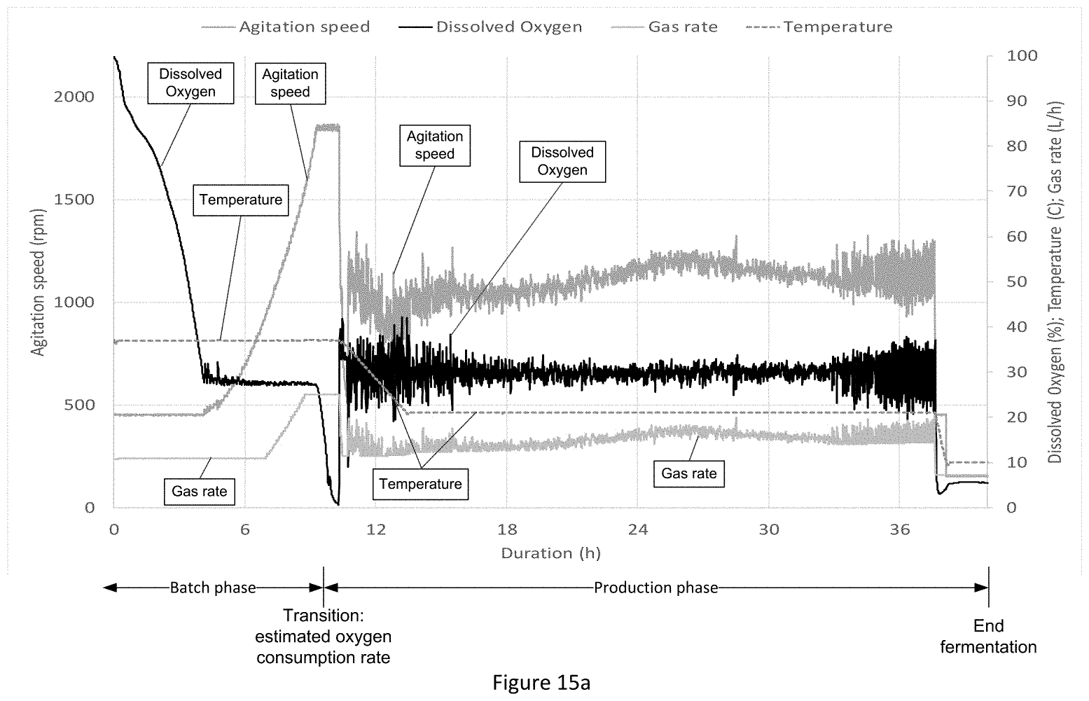

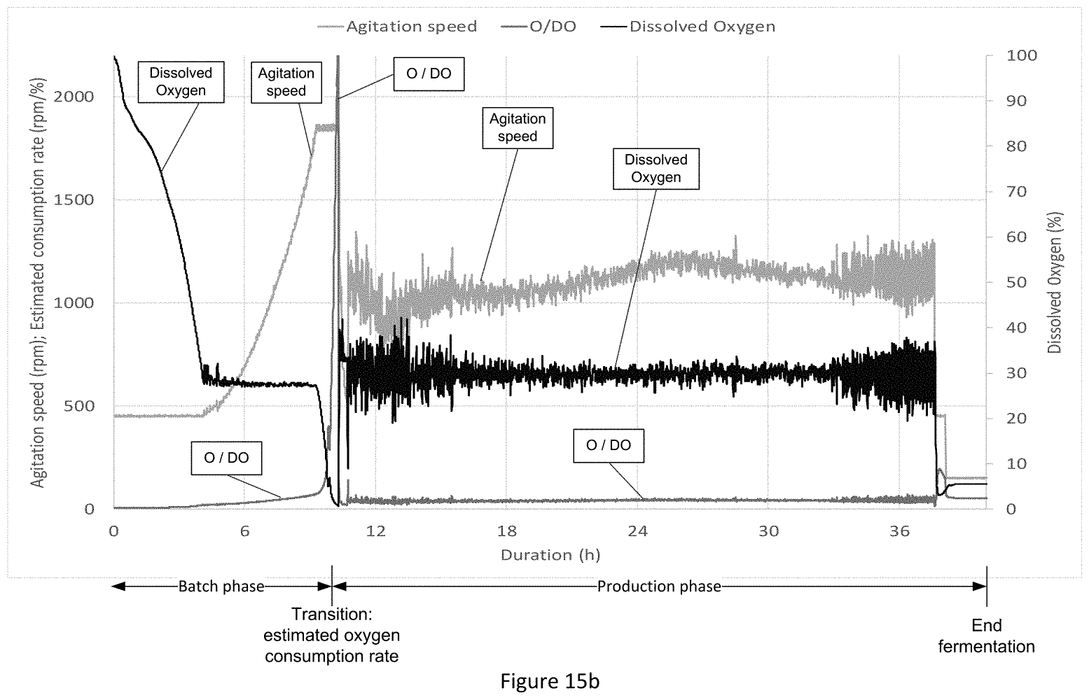

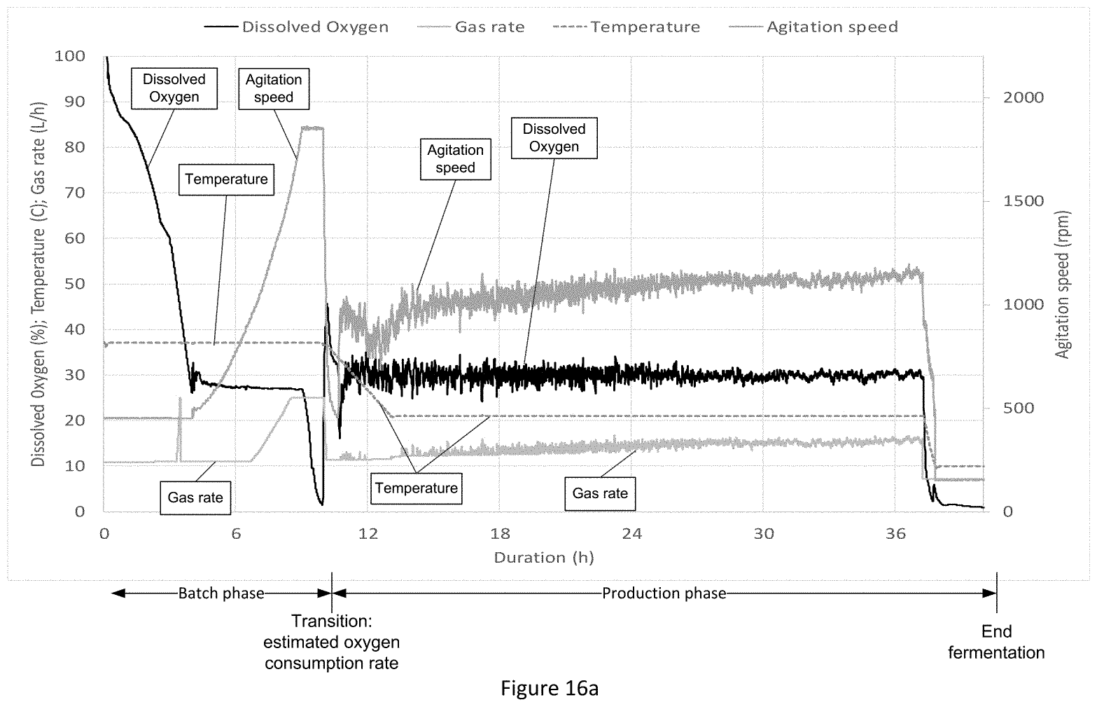

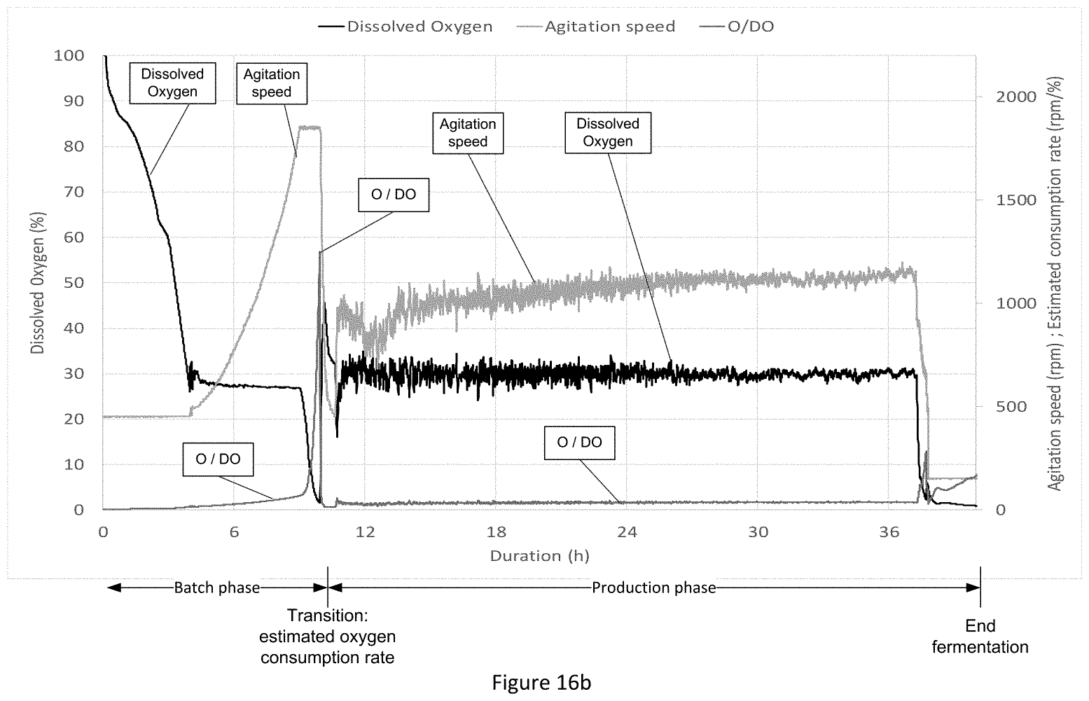

FIGS. 15a and 15b, and 16a and 16b show agitation rate, measured dissolved oxygen, gas rate, measured temperature, and estimated oxygen consumption (“O/DO”; as determined using the ratio between the agitation rate (N) and the measured DO using equation (4) above) over the course of the same bioprocess controlled by the control method illustrated in FIGS. 5-11 and as described above. In particular, in the example illustrated in FIGS. 15a and 15b the agitation system stirring rate/speed (N) in dependence on DO is controlled using equation (2a) above, without a vessel scaling factor (the 1^{st} control method), and in the example illustrated in FIGS. 16a and 16b the agitation system stirring rate/speed (N) in dependence on DO is controlled using equation (2b) above, including a vessel scaling factor (the 2^{nd} control method).

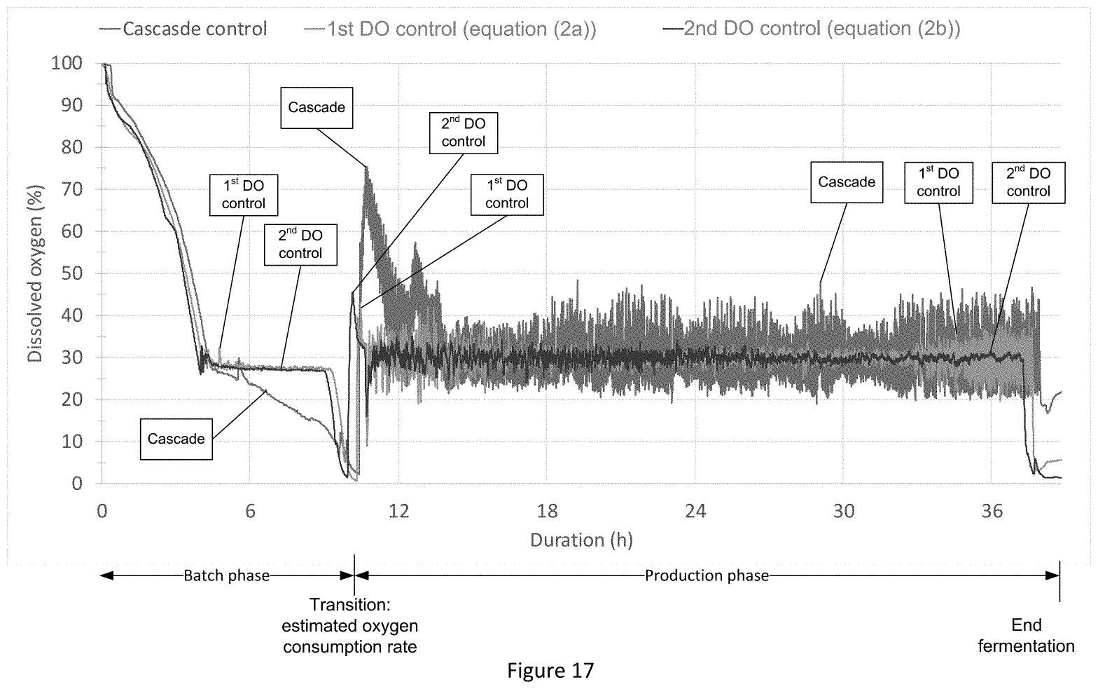

FIG. 17 shows measured dissolved oxygen over the course of the same fermentation process controlled by: the conventional cascade driven DO control of FIGS. 14a and 14b; the 1^{st} control method of FIGS. 15a and 15b; and the control method of FIGS. 16a and 16b (in other words, FIG. 17 shows overlaid measured dissolved oxygen plots from FIGS. 14a/b, 15a/b, and 16a/b). Table 6 shows the mean and standard deviation in measured dissolved oxygen (DO) values for the cascade controller of FIGS. 14a/b, the 1^{st} control method of FIGS. 15a/b, and the 2^{nd} control method of FIGS. 16a/b. In Table 6, the mean and standard deviation calculations have been split between the batch phase, a transitional period between the batch phase to the production phase, and the production phase.

FIGS. 14a to 17 demonstrate similar trends to FIGS. 12 and 13. In the examples illustrated in FIGS. 15a/b and 16a/b (and the example illustrated in FIGS. 16a/b in particular) relatively stable DO control is achieved with small fluctuations or deviations. As shown in Table 6, during both the batch phase and the production phase, the control methods illustrated in FIGS. 5-11 and as described above (the 1^{st} and 2^{nd} control methods) result in a mean DO value closer to the setpoint of 30% and in a smaller standard deviation that the conventional cascade driven controller. The 2^{nd} control method for example maintains a stable DO value near the setpoint of 30% with a mean of 29.860% and standard deviation of 1.256% in the production phase. Moreover, the 1^{st} and 2^{nd} control methods are able to achieve smaller fluctuations in the DO value during the transition period (as showcased by their considerably smaller standard deviations in the transition period) than the conventional cascade driven controller.

TABLE 6
mean DO and standard deviation during various phases
of a bioprocess using different control methods.

| | | | 1^{st} control | 2^{nd} control |
|---|---|---|---|---|
| | | Cascade | method | method |
| Batch phase DO (%) | Mean | 22.177 | 27.929 | 27.528 |
| (Duration ~4:00:00- | Standard | 5.469 | 0.894 | 0.871 |
| 9:00:00) | deviation | | | |
| ~Transition period DO (%) | Mean | 32.183 | 23.315 | 25.100 |
| (Duration ~9:00:00- | Standard | 24.375 | 11.279 | 11.277 |
| 12:00:00) | deviation | | | |
| Production phase DO (%) | Mean | 30.534 | 29.855 | 29.860 |
| (Duration ~12:00:00- | Standard | 6.446 | 2.171 | 1.256 |
| 36:00:00) | deviation | | | |

Comparing FIGS. 14b, 15b, and 16b, it can be seen that the DO spike is considerably less pronounced (visible) when the 1^{st} or 2^{nd} control methods are used as opposed to the cascade driven controller. Thus, conventional DO spike detection may not be a practical (and/or an accurate method) for (detecting (the onset of)) phase transitioning for the 1^{st} and 2^{nd} control methods (which, as demonstrated above, result in improved DO control). In contrast, a visible spike in estimated oxygen consumption can be seen in all of FIGS. 14b, 15b and 16b (i.e. for all three control methods). Accordingly, an estimated consumption rate (O/DO) based approach allows using improved DO controllers (control methods) without compromising phase transitioning and/or the accuracy of detecting the onset of phase transition. The O/DO based approach, as opposed to the conventional DO spike detection, is particularly useful when using the 1^{st} and 2^{nd} (Potential-Derivative (PD)) controllers. The PD controllers might cause a dampening/anticipatory effect of DO control, so using PD controllers with ordinary spike detection (e.g. DO above a threshold) may result in an unacceptable delay. In other words, it may only be feasible to use a PD controller (which achieves smoother control) because an alternative, estimated oxygen consumption (e.g. O/DO) based approach for phase transitioning is used.

Table 7 shows an extract of values from FIGS. 14a and 14b (conventional cascade driven controller) showing agitation rate, measured dissolved oxygen, and estimated oxygen consumption (as determined using the ratio between the agitation rate (N) and the measured DO using equation (4) above). The conventional cascade controller of FIGS. 14a/b transitions between a batch phase and a production phase by monitoring the DO and transitioning when the DO level exceeds a predetermined value (in this case 60%, as per Table 7). Using this standard method, the conventional cascade controller began transitioning at a duration point of 10.548 h. However, the onset of carbon source exhaustion has in fact occurred before that point, which could be detected by monitoring the estimated oxygen consumption (O/DO) as described above—this method would allow transitioning at a duration point of 10.423 h (i.e. 7 minutes 30 seconds before the DO spike based method). As noted above, any delay between the actual phase transitioning and the optimum phase transition may result in inappropriate/suboptimal control of the bioreactor in the meantime (e.g. incorrect process condition setpoints being used). Thus, detecting a transition (transitioning) using the estimated oxygen consumption based method described above may allow more accurate detection of the onset of carbon source exhaustion and more effective control of the bioreactor.

extract of plotted values in transition region from FIGS.
14a and 14b with the conventional control method.

| | Dissolved | Agitation | O/DO | Transition |
|---|---|---|---|---|
| Duration (h) | Oxygen (%) | speed (rpm) | (rpm/%) | detection |
| 10.356 | 2.492 | 1847.749 | 741.605 | |
| 10.365 | 2.466 | 1848.425 | 749.429 | |
| 10.373 | 2.441 | 1851.397 | 758.355 | |
| 10.381 | 2.416 | 1851.120 | 766.122 | |
| 10.390 | 2.391 | 1852.395 | 774.700 | |
| 10.398 | 2.366 | 1847.130 | 780.697 | |
| 10.406 | 2.265 | 1852.162 | 817.732 | |
| 10.415 | 2.164 | 1844.278 | 852.254 | |
| 10.423 | 12.625 | 1843.526 | 146.022 | Transition: |
| | | | | O/DO based |
| 10.431 | 33.597 | 1851.598 | 55.112 | |
| 10.440 | 46.170 | 1843.972 | 39.939 | |
| 10.448 | 53.207 | 1853.035 | 34.827 | |
| 10.456 | 57.273 | 1851.789 | 32.333 | |
| 10.465 | 45.766 | 1850.445 | 40.433 | |
| 10.473 | 45.236 | 1844.175 | 40.768 | |
| 10.481 | 51.291 | 1850.495 | 36.078 | |
| 10.490 | 43.329 | 1848.567 | 42.664 | |
| 10.498 | 44.150 | 1850.940 | 41.924 | |
| 10.506 | 48.976 | 1853.560 | 37.846 | |
| 10.515 | 58.387 | 1852.776 | 31.733 | |
| 10.523 | 49.000 | 1850.049 | 37.756 | |
| 10.531 | 52.892 | 1853.202 | 35.037 | |
| 10.540 | 51.035 | 1851.740 | 36.284 | |
| 10.548 | 62.142 | 1854.726 | 29.847 | Transition: |
| | | | | DO spike based |
| 10.556 | 53.518 | 1854.494 | 34.652 | |
| 10.565 | 62.512 | 1850.809 | 29.607 | |
| 10.573 | 54.378 | 1855.625 | 34.125 | |

As shown in FIGS. 15a, 16a, 18, and 19, towards the end of the batch phase, the gas rate reaches its maximum permitted before the agitation speed does. When the gas rate reaches the maximum value, the agitation speed is at approximately 80% of its maximum permitted value. Thus, in the agitation speed range 80%-100% (of the maximum value) the gas supply remains maximal i.e. at 100% of the maximum value. This may allow controlling DO more precisely in the late stage of batch phase as only one parameter is changing (i.e. agitation speed), so that any interactions between gassing and agitation (and corresponding synergistic effects) are eliminated, which may reduce overshooting. Further, ‘maxing out’ the gas rate prior to agitation speed may reduce the probability of spills as the aeration is subject to less change.

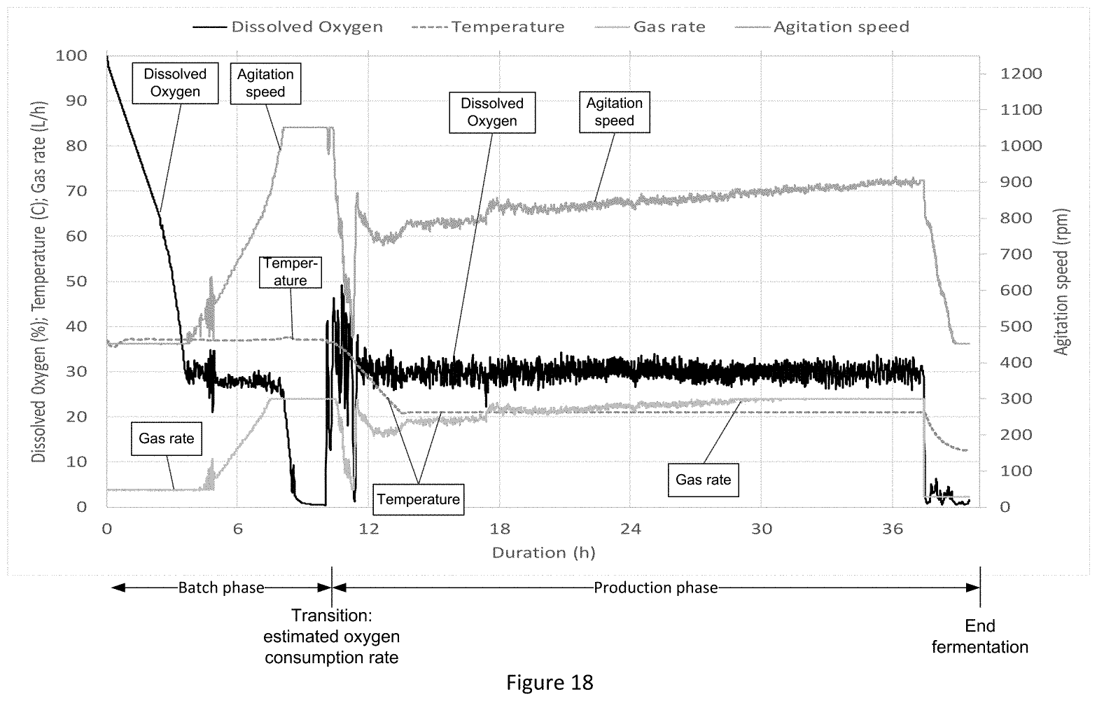

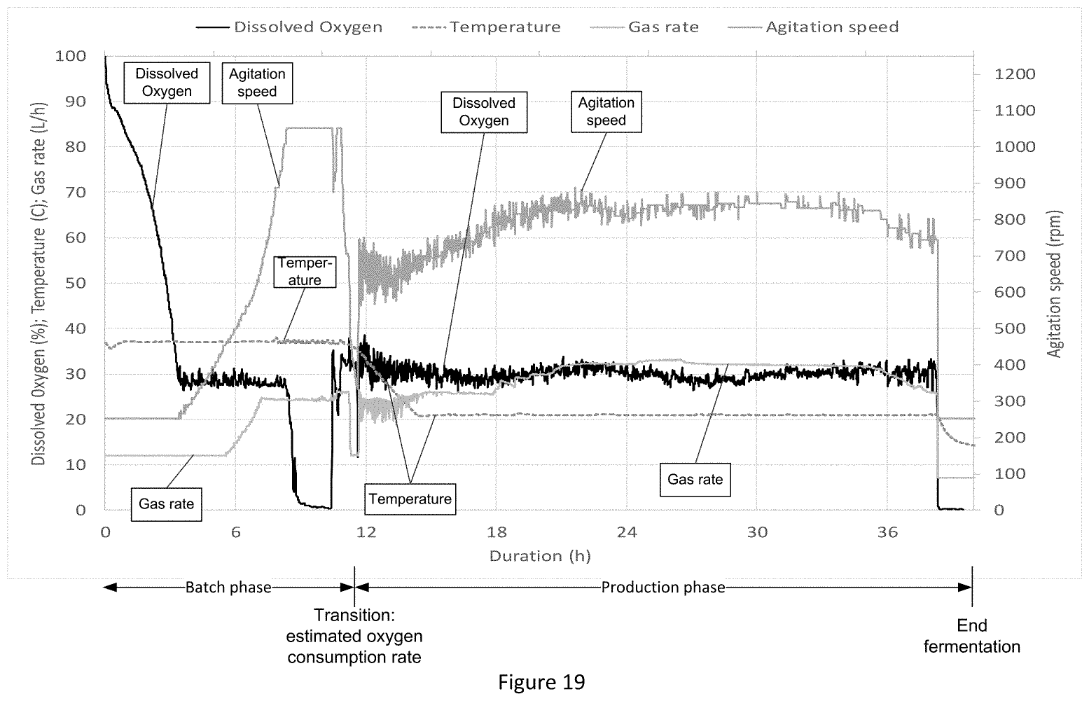

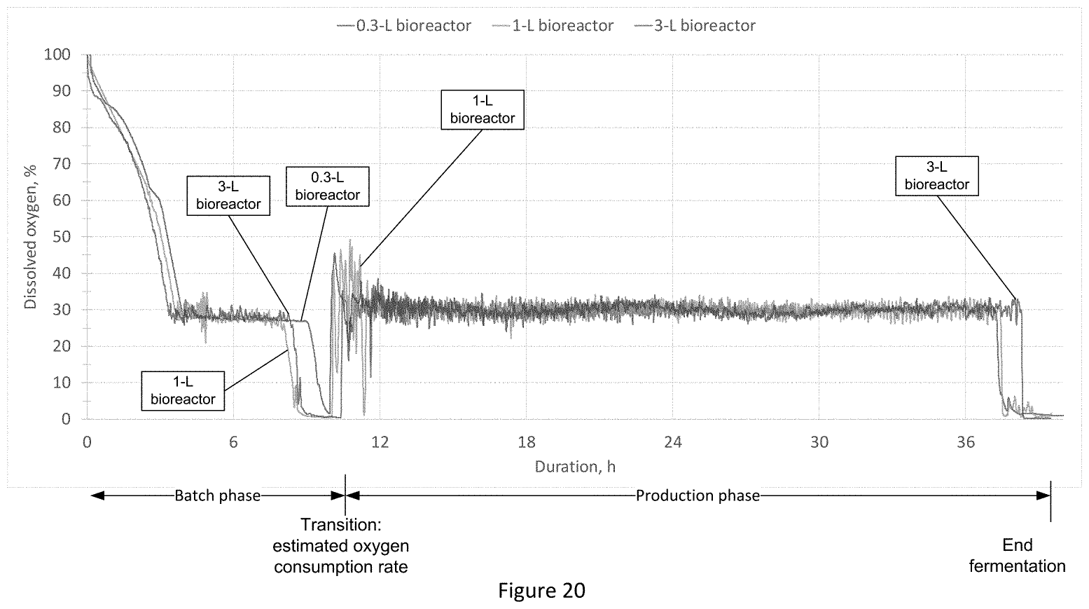

FIGS. 14a, 14b, 15a, 15b, 16a, and 16b show process conditions as measured for a fermentation process in a vessel with a 0.3 L working volume (Eppendorf® DASbox®). FIGS. 17 and 18 show the same process conditions for the same fermentation process in a vessel with a 1 L working volume (Eppendorf® BioFlo® 1 L) (FIG. 18) and in a vessel with a 3 L working volume (Eppendorf® BioFlo® 3 L) (FIG. 19) controlled using the 2^{nd} control method of FIG. 16a. FIG. 20 shows measured dissolved oxygen over the course of the same fermentation process in: a vessel with a 0.3 L working volume of FIG. 16a; a vessel with a 1 L working volume of FIG. 18; and a vessel with a 3 L working volume of FIG. 19 (in other words, FIG. 20 shows overlaid measured dissolved oxygen plots from FIGS. 16a, 18 and 19). Thus, FIGS. 18 to 20 show that the above describe control method is scalable across a range of vessel sizes.

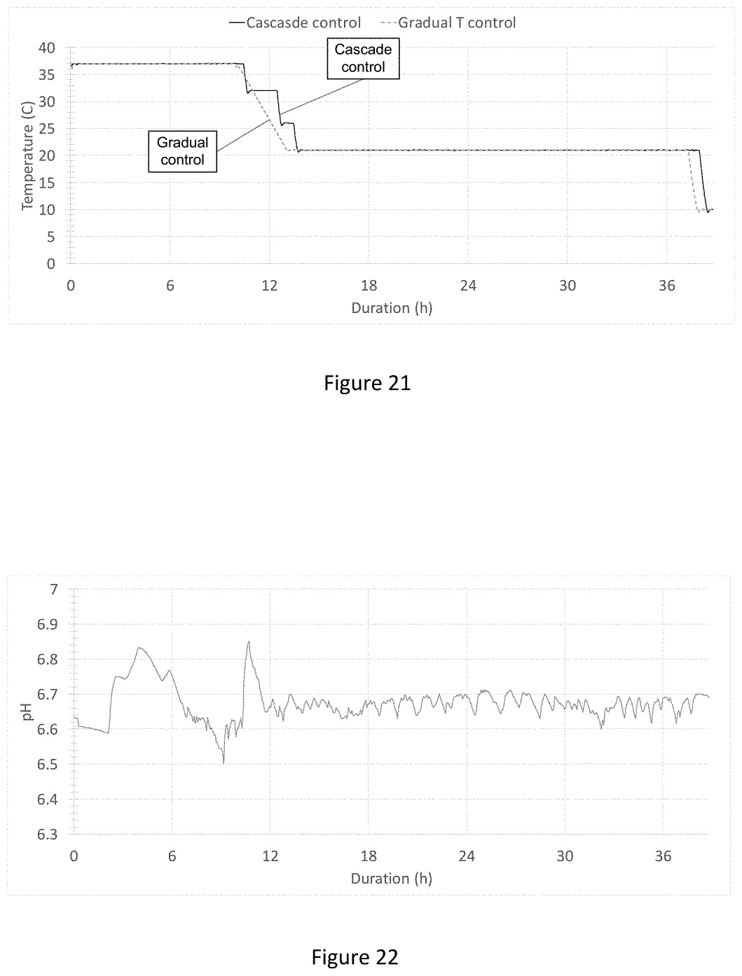

FIG. 21 shows measured temperature over the course of a fermentation process as extracted from FIGS. 14a (“Cascaded control”) and 16a (“Gradual T control”). For the cascade controller, the temperature transition between the batch phase and production phase setpoints is noticeably staggered with periods of no change interlaced with periods with a high temperature gradient. As described above, such high gradients may lead to the creation of cold spots at the interface between the vessel and the cooling system which may lead to cell damage/death. In contrast, the controller of FIG. 16a achieves a constant temperature gradient (which is considerably lower than the high gradients for the cascade controller) and thus overcomes this potential problem.

FIG. 22 shows measured pH over the course of the same fermentation process and DO control method as for FIGS. 16a and 16b. The pH is controlled by the control method illustrated in FIGS. 5-11 and as described above. The pH setpoint was set to 6.67. As shown in FIG. 22, the pH controller achieves a smooth and responsive control. Over the entire fermentation process, the mean pH is 6.673 (with a standard deviation of 0.051). During the production phase (duration-12:00:00-36:00:00), yet closer control is achieved with a mean pH of 6.669 (with a standard deviation of 0.020).

In an exemplary bioprocess a protein is produced by recombinant E. coli. Briefly, DNA constructs encoding the desired protein are synthesised, cloned into a suitable vector and then transformed into a suitable strain of E. coli cells for over-expression. The E. coli is cultivated in a fed batch fermentation bioprocess as described above for over-expression of the desired recombinant proteins. The recombinant protein can be purified from the E. coli lysates using conventional techniques. Such a bioprocess can be used for production of a wide variety of biomolecules, including clostridial neurotoxins as described above. The control method of the present disclosure is not limited to bioprocesses based on protein expression by recombinant E. coli, or to bioprocesses for producing clostridial neurotoxins, but can be used in a wide variety of bioprocesses and for producing a wide variety of products.

### Alternative Examples and Embodiments

A person skilled in the art will appreciate that many different combinations of embodiments and examples described with reference to FIGS. 1 to 13 may be used alone unmodified or in combination with each other.

The described examples of the invention are only examples of how the invention may be implemented. Modifications, variations and changes to the described examples will occur to those having appropriate skills and knowledge. These modifications, variations and changes may be made without departure from the scope of the claims.

In particular, the bioprocess may be based on a wide variety of microorganisms, including bacteria, yeasts, and fungi. The bioprocess may be based on a wide variety of production models, including vector modified recombinant microorganisms for protein expression. The bioprocess may be based on a wide variety of medium compositions and gas supplies, including anaerobic metabolic processes. The bioprocess may be based on a wide variety of cultivation conditions, such as pH, temperature, dissolved oxygen and feed rate.

Certain of the control features, such as the DO control algorithm, can be applied to bioprocesses that are not fed batch processes, in particular to continuous cultivation processes or to batch processes.

In a variant the control strategy is adapted to implement a bioprocess with three fermentation phases, where the initial batch phase is followed by a transition phase during which the microorganisms are transitioned from one metabolic state to another, for example by changing the feed composition, and finally a production phase where the production of the desired product is performed.

Sequence Listing

Where an initial Met amino acid residue or a corresponding initial codon is indicated in any of the following SEQ ID NOs, said residue/codon is optional.

SEQ ID NO: 1-BoNT/A1, accession number P0DPI1, amino acid sequence
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELNLVI
IGPSADIIQFECKSFGHEVLNLTRNGYGSTQYIRFSPDFTFGFEESLEVDTNPLLGAGKFATDPAVTLAHELIHAGH
RLYGIAINPNRVFKVNTNAYYEMSGLEVSFEELRTFGGHDAKFIDSLQENEFRLYYYNKFKDIASTLNKAKSIVGT
TASLQYMKNVFKEKYLLSEDTSGKFSVDKLKFDKLYKMLTEIYTEDNFVKFFKVLNRKTYLNFDKAVFKINIVPK
VNYTIYDGFNLRNTNLAANFNGQNTEINNMNFTKLKNFTGLFEFYKLLCVRGIITSKTKSLDKGYNKALNDLCIK
VNNWDLFFSPSEDNFTNDLNKGEEITSDTNIEAAEENISLDLIQQYYLTFNFDNEPENISIENLSSDIIGQLELMPNIE
RFPNGKKYELDKYTMFHYLRAQEFEHGKSRIALTNSVNEALLNPSRVYTFFSSDYVKKVNKATEAAMFLGWVE
QLVYDFTDETSEVSTTDKIADITIIIPYIGPALNIGNMLYKDDFVGALIFSGAVILLEFIPEIAIPVLGTFALVSYIANK
VLTVQTIDNALSKRNEKWDEVYKYIVTNWLAKVNTQIDLIRKKMKEALENQAEATKAIINYQYNQYTEEEKNNI
NFNIDDLSSKLNESINKAMININKFLNQCSVSYLMNSMIPYGVKRLEDFDASLKDALLKYIYDNRGTLIGQVDRLK
DKVNNTLSTDIPFQLSKYVDNQRLLSTFTEYIKNIINTSILNLRYESNHLIDLSRYASKINIGSKVNFDPIDKNQIQLF
NLESSKIEVILKNAIVYNSMYENFSTSFWIRIPKYFNSISLNNEYTIINCMENNSGWKVSLNYGEIIWTLQDTQEIKQ
RVVFKYSQMINISDYINRWIFVTITNNRLNNSKIYINGRLIDQKPISNLGNIHASNNIMFKLDGCRDTHRYIWIKYFN
LFDKELNEKEIKDLYDNQSNSGILKDFWGDYLQYDKPYYMLNLYDPNKYVDVNNVGIRGYMYLKGPRGSVMT
TNIYLNSSLYRGTKFIIKKYASGNKDNIVRNNDRVYINVVVKNKEYRLATNASQAGVEKILSALEIPDVGNLSQV
VVMKSKNDQGITNKCKMNLQDNNGNDIGFIGFHQFNNIAKLVASNWYNRQIERSSRTLGCSWEFIPVDDGWGER
PL
SEQ ID NO: 2-BoNT/BL accession number B1INP5, amino acid sequence
MPVTINNFNYNDPIDNNNIIMMEPPFARGTGRYYKAFKITDRIWIIPERYTFGYKPEDFNKSSGIFNRDVCEYYDPD
YLNTNDKKNIFLQTMIKLFNRIKSKPLGEKLLEMIINGIPYLGDRRVPLEEFNTNIASVTVNKLISNPGEVERKKGIF
ANLIIFGPGPVLNENETIDIGIQNHFASREGFGGIMQMKFCPEYVSVFNNVQENKGASIFNRRGYFSDPALILMHEL
IHVLHGLYGIKVDDLPIVPNEKKFFMQSTDAIQAEELYTFGGQDPSIITPSTDKSIYDKVLQNFRGIVDRLNKVLVC
ISDPNININIYKNKFKDKYKFVEDSEGKYSIDVESFDKLYKSLMFGFTETNIAENYKIKTRASYFSDSLPPVKIKNLL
DNEIYTIEEGFNISDKDMEKEYRGQNKAINKQAYEEISKEHLAVYKIQMCKSVKAPGICIDVDNEDLFFIADKNSF
SDDLSKNERIEYNTQSNYIENDFPINELILDTDLISKIELPSENTESLTDFNVDVPVYEKQPAIKKIFTDENTIFQYLY
SQTFPLDIRDISLTSSFDDALLFSNKVYSFFSMDYIKTANKVVEAGLFAGWVKQIVNDFVIEANKSNTMDKIADISL
IVPYIGLALNVGNETAKGNFENAFEIAGASILLEFIPELLIPVVGAFLLESYIDNKNKIIKTIDNALTKRNEKWSDMY
GLIVAQWLSTVNTQFYTIKEGMYKALNYQAQALEEIIKYRYNIYSEKEKSNINIDFNDINSKLNEGINQAIDNINNF
INGCSVSYLMKKMIPLAVEKLLDFDNTLKKNLLNYIDENKLYLIGSAEYEKSKVNKYLKTIMPFDLSIYTNDTILIE
MFNKYNSEILNNIILNLRYKDNNLIDLSGYGAKVEVYDGVELNDKNQFKLTSSANSKIRVTQNQNIIFNSVFLDFS
VSFWIRIPKYKNDGIQNYIHNEYTIINCMKNNSGWKISIRGNRIIWTLIDINGKTKSVFFEYNIREDISEYINRWFFVT
ITNNLNNAKIYINGKLESNTDIKDIREVIANGEIIFKLDGDIDRTQFIWMKYFSIFNTELSQSNIEERYKIQSYSEYLK
DFWGNPLMYNKEYYMFNAGNKNSYIKLKKDSPVGEILTRSKYNQNSKYINYRDLYIGEKFIIRRKSNSQSINDDIV
RKEDYIYLDFFNLNQEWRVYTYKYFKKEEEKLFLAPISDSDEFYNTIQIKEYDEQPTYSCQLLFKKDEESTDEIGLI
GIHRFYESGIVFEEYKDYFCISKWYLKEVKRKPYNLKLGCNWQFIPKDEGWTE
SEQ ID NO: 3-BoNT/CL accession number P18640, amino acid sequence
MPITINNFNYSDPVDNKNILYLDTHLNTLANEPEKAFRITGNIWVIPDRFSRNSNPNLNKPPRVTSPKSGYYDPNYL
STDSDKDPFLKEIIKLFKRINSREIGEELIYRLSTDIPFPGNNNTPINTFDFDVDFNSVDVKTRQGNNWVKTGSINPS
VIITGPRENIIDPETSTFKLTNNTFAAQEGFGALSIISISPRFMLTYSNATNDVGEGRFSKSEFCMDPILILMHELNHA
MHNLYGIAIPNDQTISSVTSNIFYSQYNVKLEYAEIYAFGGPTIDLIPKSARKYFEEKALDYYRSIAKRLNSITTANP
SSFNKYIGEYKQKLIRKYRFVVESSGEVTVNRNKFVELYNELTQIFTEFNYAKIYNVQNRKIYLSNVYTPVTANIL
DDNVYDIQNGFNIPKSNLNVLFMGQNLSRNPALRKVNPENMLYLFTKFCHKAIDGRSLYNKTLDCRELLVKNTD
LPFIGDISDVKTDIFLRKDINEETEVIYYPDNVSVDQVILSKNTSEHGQLDLLYPSIDSESEILPGENQVFYDNRTQN
VDYLNSYYYLESQKLSDNVEDFTFTRSIEEALDNSAKVYTYFPTLANKVNAGVQGGLFLMWANDVVEDFTTNIL
RKDTLDKISDVSAIIPYIGPALNISNSVRRGNFTEAFAVTGVTILLEAFPEFTIPALGAFVIYSKVQERNEIIKTIDNCL
EQRIKRWKDSYEWMMGTWLSRIITQFNNISYQMYDSLNYQAGAIKAKIDLEYKKYSGSDKENIKSQVENLKNSL
DVKISEAMNNINKFIRECSVTYLFKNMLPKVIDELNEFDRNTKAKLINLIDSHNIILVGEVDKLKAKVNNSFQNTIP
FNIFSYTNNSLLKDIINEYFNNINDSKILSLQNRKNTLVDTSGYNAEVSEEGDVQLNPIFPFDFKLGSSGEDRGKVIV
TQNENIVYNSMYESFSISFWIRINKWVSNLPGYTIIDSVKNNSGWSIGIISNFLVFTLKQNEDSEQSINFSYDISNNAP
GYNKWFFVTVTNNMMGNMKIYINGKLIDTIKVKELTGINFSKTITFEINKIPDTGLITSDSDNINMWIRDFYIFAKE
LDGKDINILFNSLQYTNVVKDYWGNDLRYNKEYYMVNIDYLNRYMYANSRQIVFNTRRNNNDFNEGYKIIIKRI
RGNTNDTRVRGGDILYFDMTINNKAYNLFMKNETMYADNHSTEDIYAIGLREQTKDINDNIIFQIQPMNNTYYYA
SQIFKSNFNGENISGICSIGTYRFRLGGDWYRHNYLVPTVKQGNYASLLESTSTHWGFVPVSE
SEQ ID NO: 4-BoNT/D, accession number P19321, amino acid sequence
MTWPVKDFNYSDPVNDNDILYLRIPQNKLITTPVKAFMITQNIWVIPERFSSDTNPSLSKPPRPTSKYQSYYDPSYL
STDEQKDTFLKGIIKLFKRINERDIGKKLINYLVVGSPFMGDSSTPEDTFDFTRHTTNIAVEKFENGSWKVTNIITPS
VLIFGPLPNILDYTASLTLQGQQSNPSFEGFGTLSILKVAPEFLLTFSDVTSNQSSAVLGKSIFCMDPVIALMHELTH
SLHQLYGINIPSDKRIRPQVSEGFFSQDGPNVQFEELYTFGGLDVEIIPQIERSQLREKALGHYKDIAKRLNNINKTI
PSSWISNIDKYKKIFSEKYNFDKDNTGNFVVNIDKFNSLYSDLTNVMSEVVYSSQYNVKNRTHYFSRHYLPVFAN
ILDDNIYTIRDGFNLTNKGFNIENSGQNIERNPALQKLSSESVVDLFTKVCLRLTKNSRDDSTCIKVKNNRLPYVA
DKDSISQEIFENKIITDETNVQNYSDKFSLDESILDGQVPINPEIVDPLLPNVNMEPLNLPGEEIVFYDDITKYVDYL
NSYYYLESQKLSNNVENITLTTSVEEALGYSNKIYTFLPSLAEKVNKGVQAGLFLNWANEVVEDFTTNIMKKDTL
DKISDVSVIIPYIGPALNIGNSALRGNFNQAFATAGVAFLLEGFPEFTIPALGVFTFYSSIQEREKIIKTIENCLEQRVK
RWKDSYQWMVSNWLSRITTQFNHINYQMYDSLSYQADAIKAKIDLEYKKYSGSDKENIKSQVENLKNSLDVKIS
EAMNNINKFIRECSVTYLFKNMLPKVIDELNKFDLRTKTELINLIDSHNIILVGEVDRLKAKVNESFENTMPFNIFS
YTNNSLLKDIINEYFNSINDSKILSLQNKKNALVDTSGYNAEVRVGDNVQLNTIYTNDFKLSSSGDKIIVNLNNNIL
YSAIYENSSVSFWIKISKDLTNSHNEYTIINSIEQNSGWKLCIRNGNIEWILQDVNRKYKSLIFDYSESLSHTGYTNK
WFFVTITNNIMGYMKLYINGELKQSQKIEDLDEVKLDKTIVFGIDENIDENQMLWIRDFNIFSKELSNEDINIVYEG
QILRNVIKDYWGNPLKFDTEYYIINDNYIDRYIAPESNVLVLVQYPDRSKLYTGNPITIKSVSDKNPYSRILNGDNII
LHMLYNSRKYMIIRDTDTIYATQGGECSQNCVYALKLQSNLGNYGIGIFSIKNIVSKNKYCSQIFSSFRENTMLLA
DIYKPWRFSFKNAYTPVAVTNYETKLLSTSSFWKFISRDPGWVE
SEQ ID NO: 5-BoNT/E1, accession number WP_003372387, amino acid sequence
MPKINSFNYNDPVNDRTILYIKPGGCQEFYKSFNIMKNIWIIPERNVIGTTPQDFHPPTSLKNGDSSYYDPNYLQSD
EEKDRFLKIVTKIFNRINNNLSGGILLEELSKANPYLGNDNTPDNQFHIGDASAVEIKFSNGSQDILLPNVIIMGAEP
DLFETNSSNISLRNNYMPSNHGFGSIAIVTFSPEYSFRFNDNSMNEFIQDPALTLMHELIHSLHGLYGAKGITTKYTI
TQKQNPLITNIRGTNIEEFLTFGGTDLNIITSAQSNDIYTNLLADYKKIASKLSKVQVSNPLLNPYKDVFEAKYGLD
KDASGIYSVNINKFNDIFKKLYSFTEFDLATKFQVKCRQTYIGQYKYFKLSNLLNDSIYNISEGYNINNLKVNFRG
QNANLNPRIITPITGRGLVKKIIRFCKNIVSVKGIRKSICIEINNGELFFVASENSYNDDNINTPKEIDDTVTSNNNYE
NDLDQVILNFNSESAPGLSDEKLNLTIQNDAYIPKYDSNGTSDIEQHDVNELNVFFYLDAQKVPEGENNVNLTSSI
DTALLEQPKIYTFFSSEFINNVNKPVQAALFVSWIQQVLVDFTTEANQKSTVDKIADISIVVPYIGLALNIGNEAQK
GNFKDALELLGAGILLEFEPELLIPTILVFTIKSFLGSSDNKNKVIKAINNALKERDEKWKEVYSFIVSNWMTKINT
QFNKRKEQMYQALQNQVNAIKTIIESKYNSYTLEEKNELTNKYDIKQIENELNQKVSIAMNNIDRFLTESSISYLM
KLINEVKINKLREYDENVKTYLLNYIIQHGSILGESQQELNSMVTDTLNNSIPFKLSSYTDDKILISYFNKFFKRIKS
SSVLNMRYKNDKYVDTSGYDSNININGDVYKYPTNKNQFGIYNDKLSEVNISQNDYIIYDNKYKNFSISFWVRIP
NYDNKIVNVNNEYTIINCMRDNNSGWKVSLNHNEIIWTLQDNAGINQKLAFNYGNANGISDYINKWIFVTITNDR
LGDSKLYINGNLIDQKSILNLGNIHVSDNILFKIVNCSYTRYIGIRYFNIFDKELDETEIQTLYSNEPNTNILKDFWG
NYLLYDKEYYLLNVLKPNNFIDRRKDSTLSINNIRSTILLANRLYSGIKVKIQRVNNSSTNDNLVRKNDQVYINFV
ASKTHLFPLYADTATTNKEKTIKISSSGNRFNQVVVMNSVGNNCTMNFKNNNGNNIGLLGFKADTVVASTWYY
THMRDHTNSNGCFWNFISEEHGWQEK
SEQ ID NO: 6-BoNT/F1, accession number Q57236, amino acid sequence
MPVVINSFNYNDPVNDDTILYMQIPYEEKSKKYYKAFEIMRNVWIIPERNTIGTDPSDFDPPASLENGSSAYYDPN
YLTTDAEKDRYLKTTIKLFKRINSNPAGEVLLQEISYAKPYLGNEHTPINEFHPVTRTTSVNIKSSTNVKSSIILNLL
VLGAGPDIFENSSYPVRKLMDSGGVYDPSNDGFGSINIVTFSPEYEYTFNDISGGYNSSTESFIADPAISLAHELIHA
LHGLYGARGVTYKETIKVKQAPLMIAEKPIRLEEFLTFGGQDLNIITSAMKEKIYNNLLANYEKIATRLSRVNSAP
PEYDINEYKDYFQWKYGLDKNADGSYTVNENKFNEIYKKLYSFTEIDLANKFKVKCRNTYFIKYGFLKVPNLLD
DDIYTVSEGFNIGNLAVNNRGQNIKLNPKIIDSIPDKGLVEKIVKFCKSVIPRKGTKAPPRLCIRVNNRELFFVASES
SYNENDINTPKEIDDTTNLNNNYRNNLDEVILDYNSETIPQISNQTLNTLVQDDSYVPRYDSNGTSEIEEHNVVDL
NVFFYLHAQKVPEGETNISLTSSIDTALSEESQVYTFFSSEFINTINKPVHAALFISWINQVIRDFTTEATQKSTFDKI
ADISLVVPYVGLALNIGNEVQKENFKEAFELLGAGILLEFVPELLIPTILVFTIKSFIGSSENKNKIIKAINNSLMERE
TKWKEIYSWIVSNWLTRINTQFNKRKEQMYQALQNQVDAIKTVIEYKYNNYTSDERNRLESEYNINNIREELNK
KVSLAMENIERFITESSIFYLMKLINEAKVSKLREYDEGVKEYLLDYISEHRSILGNSVQELNDLVTSTLNNSIPFEL
SSYTNDKILILYFNKLYKKIKDNSILDMRYENNKFIDISGYGSNISINGDVYIYSTNRNQFGIYSSKPSEVNIAQNNDI
IYNGRYQNFSISFWVRIPKYFNKVNLNNEYTIIDCIRNNNSGWKISLNYNKIIWTLQDTAGNNQKLVFNYTQMISIS
DYINKWIFVTITNNRLGNSRIYINGNLIDEKSISNLGDIHVSDNILFKIVGCNDTRYVGIRYFKVFDTELGKTEIETLY
SDEPDPSILKDFWGNYLLYNKRYYLLNLLRTDKSITQNSNFLNINQQRGVYQKPNIFSNTRLYTGVEVIIRKNGST
DISNTDNFVRKNDLAYINVVDRDVEYRLYADISIAKPEKIIKLIRTSNSNNSLGQIIVMDSIGNNCTMNFQNNNGG
NIGLLGFHSNNLVASSWYYNNIRKNTSSNGCFWSFISKEHGWQEN
SEQ ID NO: 7-BoNT/G, accession number WP_039635782, amino acid sequence
MPVNIKNFNYNDPINNDDIIMMEPFNDPGPGTYYKAFRIIDRIWIVPERFTYGFQPDQFNASTGVFSKDVYEYYDP
TYLKTDAEKDKFLKTMIKLFNRINSKPSGQRLLDMIVDAIPYLGNASTPPDKFAANVANVSINKKIIQPGAEDQIK
GLMTNLIIFGPGPVLSDNFTDSMIMNGHSPISEGFGARMMIRFCPSCLNVFNNVQENKDTSIFSRRAYFADPALTL
MHELIHVLHGLYGIKISNLPITPNTKEFFMQHSDPVQAEELYTFGGHDPSVISPSTDMNIYNKALQNFQDIANRLNI
VSSAQGSGIDISLYKQIYKNKYDFVEDPNGKYSVDKDKFDKLYKALMFGFTETNLAGEYGIKTRYSYFSEYLPPIK
TEKLLDNTIYTQNEGFNIASKNLKTEFNGQNKAVNKEAYEEISLEHLVIYRIAMCKPVMYKNTGKSEQCIIVNNED
LFFIANKDSFSKDLAKAETIAYNTQNNTIENNFSIDQLILDNDLSSGIDLPNENTEPFTNFDDIDIPVYIKQSALKKIF
VDGDSLFEYLHAQTFPSNIENLQLTNSLNDALRNNNKVYTFFSTNLVEKANTVVGASLFVNWVKGVIDDFTSEST
QKSTIDKVSDVSIIIPYIGPALNVGNETAKENFKNAFEIGGAAILMEFIPELIVPIVGFFTLESYVGNKGHIIMTISNAL
KKRDQKWTDMYGLIVSQWLSTVNTQFYTIKERMYNALNNQSQAIEKIIEDQYNRYSEEDKMNINIDFNDIDFKLN
QSINLAINNIDDFINQCSISYLMNRMIPLAVKKLKDFDDNLKRDLLEYIDTNELYLLDEVNILKSKVNRHLKDSIPF
DLSLYTKDTILIQVFNNYISNISSNAILSLSYRGGRLIDSSGYGATMNVGSDVIFNDIGNGQFKLNNSENSNITAHQS
KFVVYDSMFDNFSINFWVRTPKYNNNDIQTYLQNEYTIISCIKNDSGWKVSIKGNRIIWTLIDVNAKSKSIFFEYSI
KDNISDYINKWFSITITNDRLGNANIYINGSLKKSEKILNLDRINSSNDIDFKLINCTDTTKFVWIKDFNIFGRELNAT
EVSSLYWIQSSTNTLKDFWGNPLRYDTQYYLFNQGMQNIYIKYFSKASMGETAPRTNFNNAAINYQNLYLGLRFI
IKKASNSRNINNDNIVREGDYIYLNIDNISDESYRVYVLVNSKEIQTQLFLAPINDDPTFYDVLQIKKYYEKTTYNC
QILCEKDTKTFGLFGIGKFVKDYGYVWDTYDNYFCISQWYLRRISENINKLRLGCNWQFIPVDEGWTE
SEQ ID NO: 8-BoNT/D-C, accession number C6KZT4, amino acid sequence
MTWPVKDFNYSDPVNDNDILYLRIPQNKLITTPVKAFMITQNIWVIPERFSSDTNPSLSKPPRPTSKYQSYYDPSYL
STDEQKDTFLKGIIKLFKRINERDIGKKLINYLVVGSPFMGDSSTPEDTFDFTRHTTNIAVEKFENGSWKVTNIITPS
VLIFGPLPNILDYTASLTLQGQQSNPSFEGFGTLSILKVAPEFLLTFSDVTSNQSSAVLGKSIFCMDPVIALMHELTH
SLHQLYGINIPSDKRIRPQVSEGFFSQDGPNVQFEELYTFGGSDVEIIPQIERLQLREKALGHYKDIAKRLNNINKTI
PSSWSSNIDKYKKIFSEKYNFDKDNTGNFVVNIDKFNSLYSDLTNVMSEVVYSSQYNVKNRTHYFSKHYLPVFA
NILDDNIYTIINGFNLTTKGFNIENSGQNIERNPALQKLSSESVVDLFTKVCLRLTRNSRDDSTCIQVKNNTLPYVA
DKDSISQEIFESQIITDETNVENYSDNFSLDESILDAKVPTNPEAVDPLLPNVNMEPLNVPGEEEVFYDDITKDVDY
LNSYYYLEAQKLSNNVENITLTTSVEEALGYSNKIYTFLPSLAEKVNKGVQAGLFLNWANEVVEDFTTNIMKKD
TLDKISDVSAIIPYIGPALNIGNSALRGNFKQAFATAGVAFLLEGFPEFTIPALGVFTFYSSIQEREKIIKTIENCLEQR
VKRWKDSYQWMVSNWLSRITTQFNHISYQMYDSLSYQADAIKAKIDLEYKKYSGSDKENIKSQVENLKNSLDV
KISEAMNNINKFIRECSVTYLFKNMLPKVIDELNKFDLKTKTELINLIDSHNIILVGEVDRLKAKVNESFENTIPFNIF
SYTNNSLLKDMINEYFNSINDSKILSLQNKKNTLMDTSGYNAEVRVEGNVQLNPIFPFDFKLGSSGDDRGKVIVT
QNENIVYNAMYESFSISFWIRINKWVSNLPGYTIIDSVKNNSGWSIGIISNFLVFTLKQNENSEQDINFSYDISKNAA
GYNKWFFVTITTNMMGNMMIYINGKLIDTIKVKELTGINFSKTITFQMNKIPNTGLITSDSDNINMWIRDFYIFAKE
LDDKDINILFNSLQYTNVVKDYWGNDLRYDKEYYMINVNYMNRYMSKKGNGIVFNTRKNNNDFNEGYKIIIKRI
IGNTNDTRVRGENVLYFNTTIDNKQYSLGMYKPSRNLGTDLVPLGALDQPMDEIRKYGSFIIQPCNTFDYYASQL
FLSSNATTNRIGILSIGSYSFKLGDDYWFNHEYLIPVIKIEHYASLLESTSTHWVFVPASE
SEQ ID NO: 9-BoNT/F7, accession number UPI0001DE3DAC, amino acid sequence
MPVNINNFNYNDPINNTTILYMKMPYYEDSNKYYKAFEIMDNVWIIPERNIIGKKPSDFYPPISLDSGSSAYYDPN
YLTTDAEKDRFLKTVIKLFNRINSNPAGQVLLEEIKNGKPYLGNDHTAVNEFCANNRSTSVEIKESKGTTDSMLL
NLVILGPGPNILECSTFPVRIFPNNIAYDPSEKGFGSIQLMSFSTEYEYAFNDNTDLFIADPAISLAHELIHVLHGLYG
AKGVTNKKVIEVDQGALMAAEKDIKIEEFITFGGQDLNIITNSTNQKIYDNLLSNYTAIASRLSQVNINNSALNTTY
YKNFFQWKYGLDQDSNGNYTVNISKFNAIYKKLFSFTECDLAQKFQVKNRSNYLFHFKPFRLLDLLDDNIYSISE
GFNIGSLRVNNNGQNINLNSRIVGPIPDNGLVERFVGLCKSIVSKKGTKNSLCIKVNNRDLFFVASESSYNENGINS
PKEIDDTTITNNNYKKNLDEVILDYNSDAIPNLSSRLLNTTAQNDSYVPKYDSNGTSEIKEYTVDKLNVFFYLYAQ
KAPEGESAISLTSSVNTALLDASKVYTFFSSDFINTVNKPVQAALFISWIQQVINDFTTEATQKSTIDKIADISLVVP
YVGLALNIGNEVQKGNFKEAIELLGAGILLEFVPELLIPTILVFTIKSFINSDDSKNKIIKAINNALRERELKWKEVY
SWIVSNWLTRINTQFNKRKEQMYQALQNQVDGIKKIIEYKYNNYTLDEKNRLKAEYNIYSIKEELNKKVSLAMQ
NIDRFLTESSISYLMKLINEAKINKLSEYDKRVNQYLLNYILENSSTLGTSSVQELNNLVSNTLNNSIPFELSEYTND
KILISYFNRFYKRIIDSSILNMKYENNRFIDSSGYGSNISINGDIYIYSTNRNQFGIYSSRLSEVNITQNNTIIYNSRYQ
NFSVSFWVRIPKYNNLKNLNNEYTIINCMRNNNSGWKISLNYNNIIWTLQDTTGNNQKLVFNYTQMIDISDYINK
WTFVTITNNRLGHSKLYINGNL
TDQKSILNLGNIHVDDNILFKIVGCNDTRYVGIRYFKIFNMELDKTEIETLYHSEPDSTILKDFWGNYLLYNKKYY
LLNLLKPNMSVTKNSDILNINRQRGIYSKTNIFSNARLYTGVEVIIRKVGSTDTSNTDNFVRKNDTVYINVVDGNS
EYQLYADVSTSAVEKTIKLRRISNSNYNSNQMIIMDSIGDNCTMNFKTNNGNDIGLLGFHLNNLVASSWYYKNIR
NNTRNNGCFWSFISKEHGWQE
SEQ ID NO: 10-BoNT/X, accession number P0DPK1, amino acid sequence
MKLEINKFNYNDPIDGINVITMRPPRHSDKINKGKGPFKAFQVIKNIWIVPERYNFTNNTNDLNIPSEPIMEADAIY
NPNYLNTPSEKDEFLQGVIKVLERIKSKPEGEKLLELISSSIPLPLVSNGALTLSDNETIAYQENNNIVSNLQANLVI
YGPGPDIANNATYGLYSTPISNGEGTLSEVSFSPFYLKPFDESYGNYRSLVNIVNKFVKREFAPDPASTLMHELVH
VTHNLYGISNRNFYYNFDTGKIETSRQQNSLIFEELLTFGGIDSKAISSLIIKKIIETAKNNYTTLISERLNTVTVEND
LLKYIKNKIPVQGRLGNFKLDTAEFEKKLNTILFVLNESNLAQRFSILVRKHYLKERPIDPIYVNILDDNSYSTLEGF
NISSQGSNDFQGQLLESSYFEKIESNALRAFIKICPRNGLLYNAIYRNSKNYLNNIDLEDKKTTSKTNVSYPCSLLN
GCIEVENKDLFLISNKDSLNDINLSEEKIKPETTVFFKDKLPPQDITLSNYDFTEANSIPSISQQNILERNEELYEPIRN
SLFEIKTIYVDKLTTFHFLEAQNIDESIDSSKIRVELTDSVDEALSNPNKVYSPFKNMSNTINSIETGITSTYIFYQWL
RSIVKDFSDETGKIDVIDKSSDTLAIVPYIGPLLNIGNDIRHGDFVGAIELAGITALLEYVPEFTIPILVGLEVIGGELA
REQVEAIVNNALDKRDQKWAEVYNITKAQWWGTIHLQINTRLAHTYKALSRQANAIKMNMEFQLANYKGNID
DKAKIKNAISETEILLNKSVEQAMKNTEKFMIKLSNSYLTKEMIPKVQDNLKNFDLETKKTLDKFIKEKEDILGTN
LSSSLRRKVSIRLNKNIAFDINDIPFSEFDDLINQYKNEIEDYEVLNLGAEDGKIKDLSGTTSDINIGSDIELADGREN
KAIKIKGSENSTIKIAMNKYLRFSATDNFSISFWIKHPKPTNLLNNGIEYTLVENFNQRGWKISIQDSKLIWYLRDH
NNSIKIVTPDYI
AFNGWNLITITNNRSKGSIVYVNGSKIEEKDISSIWNTEVDDPIIFRLKNNRDTQAFTLLDQFSIYRKELNQNEVVK
LYNYYFNSNYIRDIWGNPLQYNKKYYLQTQDKPGKGLIREYWSSFGYDYVILSDSKTITFPNNIRYGALYNGSKV
LIKNSKKLDGLVRNKDFIQLEIDGYNMGISADRFNEDTNYIGTTYGTTHDLTTDFEIIQRQEKYRNYCQLKTPYNIF
HKSGLMSTETSKPTFHDYRDWVYSSAWYFQNYENLNLRKHTKTNWYFIPKDEGWDED
SEQ ID NO: 11-BoNT/A1, accession number P0DPI0, amino acid sequence
MPFVNKQFNYKDPVNGVDIAYIKIPNVGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELNLVI
IGPSADIIQFECKSFGHEVLNLTRNGYGSTQYIRFSPDFTFGFEESLEVDTNPLLGAGKFATDPAVTLAHELIHAGH
RLYGIAINPNRVFKVNTNAYYEMSGLEVSFEELRTFGGHDAKFIDSLQENEFRLYYYNKFKDIASTLNKAKSIVGT
TASLQYMKNVFKEKYLLSEDTSGKFSVDKLKFDKLYKMLTEIYTEDNFVKFFKVLNRKTYLNFDKAVFKINIVPK
VNYTIYDGFNLRNTNLAANFNGQNTEINNMNFTKLKNFTGLFEFYKLLCVRGIITSKTKSLDKGYNKALNDLCIK
VNNWDLFFSPSEDNFTNDLNKGEEITSDTNIEAAEENISLDLIQQYYLTFNFDNEPENISIENLSSDIIGQLELMPNIE
RFPNGKKYELDKYTMFHYLRAQEFEHGKSRIALTNSVNEALLNPSRVYTFFSSDYVKKVNKATEAAMFLGWVE
QLVYDFTDETSEVSTTDKIADITIIIPYIGPALNIGNMLYKDDFVGALIFSGAVILLEFIPEIAIPVLGTFALVSYIANK
VLTVQTIDNALSKRNEKWDEVYKYIVTNWLAKVNTQIDLIRKKMKEALENQAEATKAIINYQYNQYTEEEKNNI
NFNIDDLSSKLNESINKAMININKFLNQCSVSYLMNSMIPYGVKRLEDFDASLKDALLKYIYDNRGTLIGQVDRLK
DKVNNTLSTDIPFQLSKYVDNQRLLSTFTEYIKNIINTSILNLRYESNHLIDLSRYASKINIGSKVNFDPIDKNQIQLF
NLESSKIEVILKNAIVYNSMYENFSTSFWIRIPKYFNSISLNNEYTIINCMENNSGWKVSLNYGEIIWTLQDTQEIKQ
RVVFKYSQMINISDYINRWIFVTIT
NNRLNNSKIYINGRLIDQKPISNLGNIHASNNIMFKLDGCRDTHRYIWIKYFNLFDKELNEKEIKDLYDNQSNSGIL
KDFWGDYLQYDKPYYMLNLYDPNKYVDVNNVGIRGYMYLKGPRGSVMTTNIYLNSSLYRGTKFIIKKYASGNK
DNIVRNNDRVYINVVVKNKEYRLATNASQAGVEKILSALEIPDVGNLSQVVVMKSKNDQGITNKCKMNLQDNN
GNDIGFIGFHQFNNIAKLVASNWYNRQIERSSRTLGCSWEFIPVDDGWGERPL

1. A method of controlling operation of a fed batch process in a bioreactor vessel, comprising transitioning from a batch phase to a production phase in dependence on a relationship between an oxygen supply parameter O and a dissolved oxygen value DO.

2. A method according to claim 1, wherein the oxygen supply parameter O is determined in dependence on one or more of: an agitation speed; a gas supply rate; and an oxygen supply concentration.

3. A method according to claim 2, wherein the oxygen supply parameter O is a sum or product of two or more of: an agitation speed value, a gas supply rate value, and an oxygen supply concentration value.

4. A method according to any preceding claim, wherein the relationship is a ratio
$$\frac{O}{D O} .$$

5. A method according to claim 4, comprising transitioning from a batch phase to a production phase when the ratio
$$\frac{O}{D O}$$
falls below a threshold k.

6. A method according to claim 5, comprising transitioning from a batch phase to a production phase when the ratio
$$\frac{O}{D O}$$
falls below a threshold k only if the ratio
$$\frac{O}{D O}$$
has previously exceeded a threshold j.

7. A method according to claim 6, comprising transitioning from a batch phase to a production phase when the ratio
$$\frac{O}{D O}$$
falls below a threshold k only if the ratio
$$\frac{O}{D O}$$
has previously exceeded a threshold j for at least a predetermined period of time.

8. A method according to any preceding claim, wherein the process is a protein expression process and a target protein expressed in the production phase is a recombinant protein.

9. A method according to claim 8, wherein the recombinant protein is a botulinum neurotoxin.

10. A method according to any preceding claim, further comprising: controlling one or more actuators to provide a first set of process conditions during the batch phase; and controlling the actuators to provide a second set of process conditions during the production phase, with the first and second set of process conditions being different at least in part.

11. A method according to any preceding claim, wherein transitioning from the batch phase to the production phase comprises changing one or more process condition setpoints.

12. A method according to claim 11, wherein the one or more process condition setpoints include one or more of: a dissolved oxygen setpoint; and a temperature setpoint.

13. A method according to claim 11 or 12, wherein transitioning from the batch phase to the production phase comprises one or more of: providing feed to the bioreactor vessel; and providing inducer to the bioreactor vessel.

14. A method according to any of claims 11 to 13, wherein transitioning from the batch phase to the production phase comprises gradually changing over a predetermined period of time one or more process condition setpoints from a batch phase setpoint to a production phase setpoint.

15. A method according to any preceding claim, further comprising controlling a gas supply rate and/or an oxygen supply concentration proportional to the agitation speed at least in a range of the agitation.

16. A method according to any preceding claim, further comprising controlling an agitation speed in dependence on a rate of change of dissolved oxygen.

17. A method according to any preceding claim, further comprising gradually transitioning over a predetermined period of time from the production phase to a termination phase.

18. A method according to claim 17, wherein the transitioning comprises one or more of: reducing a feed supply and/or an oxygen supply to the bioreactor vessel; transitioning one or more process conditions from a production phase setpoint to a termination setpoint; and reducing agitation in the bioreactor vessel.

19. A method according to claim 17 or 18, wherein the transitioning comprises reducing a feed supply to the bioreactor vessel and transitioning a temperature from a production phase setpoint to a termination setpoint, wherein the temperature is transitioned at a rate proportional to the rate at which the feed supply is reduced.

20. A method according to any of claims 17 to 19, wherein the transitioning comprises first reducing a feed supply to the bioreactor vessel and transitioning a temperature from a production phase setpoint to a termination setpoint, and then reducing an agitation.

21. A computer programme product comprising instructions which, when executed by a computer, cause the computer to control operation of a fed batch process in a bioreactor vessel, comprising:
determining a relationship, preferably a ratio
$$\frac{O}{D O} ,$$
between an oxygen supply parameter O and a dissolved oxygen value DO; and
transitioning from a batch phase to a production phase in dependence on the relationship, and preferably the ratio
$$\frac{O}{D O} .$$

22. A computer programme product according to claim 21, comprising instructions which, when executed by a computer, cause the computer to control operation of a fed batch process in a bioreactor vessel according to the method of any of claims 1 to 20.

23. A device adapted to control operation of a fed batch process in a bioreactor vessel, the device comprising:
means for determining a relationship, preferably a ratio
$$\frac{O}{D O} ,$$
between an oxygen supply parameter O and a dissolved oxygen value DO; and
a control output adapted to transition the process from a batch phase to a production phase in dependence on the relationship, and preferably the ratio
$$\frac{O}{D O} .$$

24. A device according to claim 23, further comprising means adapted to control operation of a fed batch process in a bioreactor vessel according to the method of any of claims 1 to 20.

25. A method of controlling operation of a bioprocess in a bioreactor vessel, comprising adapting an agitation in dependence on a rate of change of dissolved oxygen.

26. A method according to claim 25, wherein the agitation is adapted in dependence on a dissolved oxygen setpoint value, a first dissolved oxygen measured value, a second, preceding, dissolved oxygen measured value, and optionally a difference between measurement times of the first and second dissolved oxygen values.

27. A method according to claim 25 or 26, further comprising adapting an oxygen supply proportional to the agitation at least in a range of the agitation.

28. A method of controlling operation of a bioprocess in a bioreactor vessel, comprising adapting an acid supply and/or a base supply in dependence on a current pH value, a preceding pH value, and a pH setpoint.

29. A method according to claim 28, wherein the acid supply and/or base supply is adapted to increase exponentially with a time during which a measured pH is not at the pH setpoint.

30. A method according to claim 28 or 29, wherein the acid supply and/or base supply is adapted to increase exponentially with a time during which a measured pH tends away from the pH setpoint.

31. A method according to any of claims 28 to 30, wherein the acid supply and/or base supply is scaled in dependence on the difference between the current pH value and the pH setpoint.

32. A method according to any of claims 1 to 20, further comprising adapting an agitation and an oxygen supply simultaneously or near-simultaneously in dependence on a dissolved oxygen setpoint and a measured dissolved oxygen.

33. A method of controlling operation of a bioprocess in a bioreactor vessel, comprising adapting an agitation and an oxygen supply simultaneously or near-simultaneously in dependence on a dissolved oxygen setpoint and a measured dissolved oxygen.

34. A method according to claim 32 or 33, wherein the oxygen supply is proportional to the agitation at least in a range of the agitation.

35. A method according to any of claims 32 to 34, wherein adapting the oxygen supply comprises adapting a volumetric flow rate of a gas supply and/or adapting an oxygen concentration of a gas supply.

36. A method according to any of claims 32 to 35, wherein a minimum oxygen supply corresponds to a minimum agitation and a maximum oxygen supply corresponds to a maximum agitation.

37. A method according to any of claims 32 to 36, wherein when an agitation increases from a minimum agitation the oxygen supply increases from the minimum oxygen supply simultaneously or near-simultaneously.

38. A method according to any of claims 32 to 37, wherein the oxygen supply reaches maximum oxygen supply before the agitation reaches maximum agitation; preferably wherein, when the oxygen supply reaches the maximum oxygen supply, the agitation is between 75% and 85% of the maximum agitation, more preferably approximately 80%.
